(19) United States
(12) Patent Application Publication
(10) Pub. No.: US 2017/0037431 A1
(43) Pub. Date: Feb. 9, 2017

(54) In vivo Gene Engineering with Adenoviral Vectors

(71) Applicant: UNIVERSITY OF WASHINGTON, Seattle, WA (US)

(72) Inventors: Andre LIEBER, Seattle, WA (US); Thalia PAPAYANNOPOULOU, Seattle, WA (US); Maximilian RICHTER, Seattle, WA (US); Kamola SAYDAMINOVA, Seattle, WA (US)

(21) Appl. No.: 15/305,300

(22) PCT Filed: May 1, 2015
(86) PCT No.: PCT/US2015/028789

§ 371 Date: Oct. 19, 2016

Related U.S. Application Data
(60) Provisional application No. 61/987,340, filed on May 1, 2014.

(51) Int. Cl. C12N 15/86; A61K 48/00; A61K 35/761; C12N 7/00; C12N 15/113

(52) U.S. Cl. CPC C12N 15/86; C12N 7/00; C12N 15/113; A61K 35/761; A61K 48/0058; A61K 48/0091; C12N 2310/141; C12N 2710/10045; C12N 2710/10052; C12N 2810/6018; C12N 2840/102

(57) ABSTRACT

The present invention provides recombinant nucleic acid expression cassetie and helper dependent adenovirus, where the expression cassettes utilize a miRNA based system for controlling expression of nucleases in helper dependent adenoviral viral producer cells, thus permitting production and use for in in vivo gene editing in CD34+ cells.

### CROSS REFERENCE

This application claims priority to U.S. Provisional Patent Application Ser. No. 61/987,340 filed May 1, 2014, incorporated by reference herein in its entirety.

### STATEMENT OF GOVERNMENT RIGHTS

This invention was made with government support under Grant No. R01 HLA078836, and R21 CA193077 awarded by the National Institutes of Health. The government has certain rights in the invention.

### BACKGROUND OF THE INVENTION

Hematopoietic stem cells (HSCs) are an important target for gene therapy. Current protocols involve the collection of HSCs from donors/patients, in vitro culture, transduction with retrovirus vectors, and retransplantation into myelo-conditioned patients. Besides its technical complexity, disadvantages of this approach include the necessity for culture in the presence of multiple cytokines which can affect the pluripotency of HSCs and their engraftment potential. Furthermore, the requirement for myeloablative regimens in patients with non-malignant disorders creates additional risks.

A major task in HSC gene therapy is the site-specific modification of the HSC genome using artificial site-specific endonucleases (EN) that target a DNA break to preselected genomic sites. ENs are employed to knock-out genes, correct frame shift mutations, or to knock-in a wild-type cDNA into the endogenous site or heterologous sites. However, none of the current EN gene delivery platforms to generate site-specific DNA breaks in the genome is adequate for in vivo engineering of mobilized HSCs.

### SUMMARY OF THE INVENTION

In a first aspect, the invention provides recombinant nucleic acid expression cassettes, comprising at least one first nucleic acid module comprising
(i) a first coding region encoding a nuclease capable of generating a DNA break in a CD34+ cell genomic target of interest; and
(ii) a second coding region encoding one or more miRNA target sites located in a 3′ untranslated region of the first coding region and at least 60 nucleotides downstream of a translation at stop codon of the first coding region, wherein miRNAs that bind to the one or more encoded miRNA target sites are highly expressed in virus producer cells but not expressed, or expressed at low levels, in CD34+ cells,
wherein the first nucleic acid module is operatively linked to a promoter that is active in CD34+ cells

In one embodiment, the cassette further comprises a second nucleic acid nodule encoding a CD46 binding adenoviral fiber polypeptide. In another embodiment, the expression cassette further comprises an inverted terminal repeat (ITR) at each terminus of the recombinant nucleic acid vector, wherein the ITR derived from a CD46-binding adenovirus serotype. In a further embodiment, the expression cassette further comprises a packaging signal from a CD46-binding adenovirus serotype.

In one embodiment, the the one or more the miRNA target site comprise a reverse complement of one, two, or all three miRNA selected from the group consisting of (a) CACUGGUAGA (SEQ ID NO: 1) (has-miR183-5p core), (b) UGUGCUUGAUCUAA (SEQ ID NO: 2) (has-miR218-5p core); and (c) CACUAGCACA (SEQ ID NO: 3) (miR96-5p core). In another embodiment, the one or miRNA target sites comprise a reverse complement of a miRNA selected from the group consisting of SEQ ID NOS: 1-90. In a further embodiment the second coding region encodes at least 4 miRNA target sites. In another embodiment, a spacer sequence of between 1-10 nucleotides is present between each encoded miRNA target site. In a still further embodiment, the nuclease is selected from the group consisting of zinc-finger nucleases (ZFNs), transcription activator-like effector nucleases (TALENs), meganucleases, and CRISPR-Cas9 nucleases, including but not limited to a nuclease comprising the amino acid sequence of a polypeptide selected from the group consisting of SEQ ID NOS 91-93. In another embodiment, the nuclease is capable of generating a DNA break in a CD34+ cell genomic target selected from the group consisting of genes encoding Chemokine Receptor Type 5 (CCR5), β-globin, Complement receptor 2 (CR2) (Epstein Barr Virus (EBV) receptor), Niemann-Pick disease, type C1 receptor ((NPC1) Ebola receptor), angiotensin-converting enzyme 2 receptor ((ACE2) SARS receptor), and genes that encode proteins that can lead to lysosomal storage disease if misfolded. In one embodiment, the promoter is selected from the group consisting of an EF1α promoter, a phosphoglycerate kinase (PGK) 1 promoter, and a ubiquitin gene promoter.

In another embodiment, the second nucleic acid module encodes an adenoviral fiber polypeptide comprising one or more human adenoviral knob domain, or equivalents thereof, that bind to CD46. In a further embodiment the knob domain is selected from the group consisting of an Ad11 knob domain, an Ad16 knob domain, an Ad21 knob domain, an Ad35 knob domain, an Ad50 knob domain, and functional equivalents thereof. In another embodiment, the knob domain is selected from the group consisting of SEQ ID NOS: 94-101. In a further embodiment, the second nucleic acid module encodes an adenoviral fiber polypeptide comprising one or more human adenoviral shaft domain or functional equivalents thereof. In one embodiment, the one or more human adenoviral shaft domains are selected from the group consisting of one or more Ad5 shaft domains, one or more Ad11 shaft domains, one or more Ad16 shaft domains, one or more Ad21 shaft domains, one or more Ad35 shaft domains, one or more Ad50 shaft domains, combinations thereof, and functional equivalents thereof. In another embodiment, the one or more human adenoviral shaft domains are selected from the group consisting of SEQ ID NOS 118-130, and 152-156.

In a further embodiment, the second nucleic acid module encodes an adenoviral fiber polypeptide comprising a human adenoviral tail domain, or equivalent thereof. In one embodiment, the human adenoviral tail domain is selected from the group consisting of an Ad11 tail domain, an Ad16 tail domain, an Ad21 tail domain, an Ad35 tail domain, an Ad50 tail domain, and functional equivalents thereof. In another embodiment, the human adenoviral tail domain is selected from the group consisting of SEQ ID NOS: 131-132. In a further embodiment, the ITRs are from Ad11, Ad16, Ad21, Ad35, or Ad50, including but not limited to a polynucleotide selected from the group consisting of SEQ ID NOS: 133-137. In another embodiment, the packaging signal comprises an Ad11, Ad16, Ad21, Ad35, of Ad50 packaging signal, including but not limited to a polynucleotide selected from the group consisting of SEQ ID NO: 138-141. In one further embodiment, the packaging signal is flanked by nucleic acid excision signals. In a still further embodiment, the cassette encodes no other adenoviral proteins.

In another embodiment, the expression cassette further comprises a transgene operatively linked to a second promoter that is active in CD34+ cells. In one embodiment, the cassette further comprises at least a first recombination site and a second recombination site flanking the transgene, wherein the first recombination site and a second recombination site target a site in CD34+ cell genomic DNA flanking a desired insertion site for the transgene. In various non-limiting embodiments, the transgene can be selected from the group consisting of -CCR5, β-globin, Complement receptor 2 (CR2) (Epstein Barr Virus (EBV) receptor), Niemann-Pick disease, type C1 receptor (NPC1) Ebola receptor), angiotensin-converting enzyme 2 receptor (ACE2) SARS receptor), and genes that encode proteins that can lead to lysosomal storage disease if misfolded.

In another aspect, the invention provides recombinant nucleic acid vectors comprising a recombinant nucleic acid expression cassette of any embodiment or combination of embodiments of the invention. In one embodiment, the expression cassette and/or recombinant nucleic acid vector are at least 28 kb in length.

In another aspect, the invention provides recombinant host cells, comprising the expression cassette or recombinant nucleic acid vector of any embodiment or combination of embodiments of the invention. In one embodiment, the host cell produces the miRNA to which the miRNA target sites encoded by the cassette bind. In another embodiment, the host cells further comprise helper adenovirus and/or helper adenovirus vector. In various embodiments, the host cell is selected from the group consisting of human embryonic kidney (HEK) 293 cells, HEK 293-Cre cells, PerC6 cells, and HCT 116 cells.

In another aspect, the invention provides recombinant helper dependent adenoviruses comprising the expression cassette or recombinant nucleic acid vector of any embodiment or combination of embodiments of the invention, as well as methods for making the recombinant helper dependent adenoviruses.

In a further aspect, the invention provides methods for hematopoietic cell gene therapy, comprising in vivo transduction of hematopoietic cells mobilized into peripheral blood of a subject in need of hematopoietic cell gene therapy with the recombinant helper dependent Ad virus of any embodiment or combination of embodiments of the invention, wherein the nuclease targets a hematopoietic cell genomic gene to be disrupted, wherein disruption of the hematopoietic cell genomic gene provides a therapeutic benefit to the subject.

In another aspect, the invention provides methods for hematopoietic cell gene therapy, comprising in vivo transduction of hematopoietic cells mobilized into peripheral blood of a subject in need of hematopoietic cell gene therapy with the recombinant helper dependent Ad virus of any embodiment or combination of embodiments of the invention, wherein the recombinant nucleic acid expression cassette comprises a transgene operatively linked to a promoter that is active in CD34+ cells, wherein the transgene is flanked by at least a first recombination site and a second recombination site, wherein the first recombination site and a second recombination site target a site in the hematopoietic cell genomic DNA flanking a desired insertion site for the transgene, and wherein insertion of the transgene into the desired insertion site provides a therapeutic benefit to the subject.

In one embodiment of the therapeutic methods of the invention, the hematopoietic cells are mobilized into peripheral blood by administering to the subject a mobilization agent combination selected from the group consisting of Granulocyte colony stimulating factor (GCSF), Plerixafor (AMD3100; a CXCR inhibitor), POL5551 CXCR4 (C-X-C chemokine receptor type 4) antagonist), BIO5192 (small molecule inhibitor of VLA-4), and combinations thereof). In another embodiment, the subject is a human. In a further embodiment, the subject is suffering from, or is at risk of developing, a disorder selected from the group consisting of β-thalassemias, human immunodeficiency virus infection and/or acquired immunodeficiency syndrome, Ebola virus infection, Epstein-Barr virus infection, and sudden acute respiratory syndrome virus (SARS) infection. In a still further embodiment, the recombinant helper dependent Ad virus is administered by intravenous injection.

In a further aspect, the invention provides recombinant nucleic acids comprising two or more copies of a miRNA target site that comprises of the reverse complement of a nucleic acid sequence selected from the group consisting of SEQ ID NOS: 1-90. In one embodiment, the recombinant nucleic acid comprises at least 4 copies of the miRNA target site. In another embodiment, the miRNA target sites in total comprise target sites for at least two different miRNAs. In a further embodiment, a spacer sequence of between 1-10 nucleotides is present between each encoded miRNA target site. In another embodiment, the recombinant nucleic acid further comprises a coding region for a protein of interest located upstream of the two or more copies of a miRNA target site, wherein the two or more copies of a miRNA target site are located within the 3′ untranslated region of the coding region and at least 60 nucleotides downstream of the translational stop codon for the coding region. In a still further embodiment, the invention provides a nucleic acid expression vector comprising the recombinant nucleic acids of this aspect of the invention operatively linked to a promoter sequence.

### DESCRIPTION OF THE FIGURES

FIG. 1. miRNA expression profiling in 293-Cre vs CD34+ cells. a) MicroRNA log2 intensity scatterplots of CD34+ cells (Y-axis) and 293-Cre cells (X-axis). miRNAs that fulfill our selection criteria (high expression level in 293-Cre cells and absent/low expression in CD34+ cells are labeled. 293-Cre and CD34+ cells (pooled from 4 different donors) were infected with Ad vectors as described in the examples. 24 hours after infection, total RNA was isolated and hybridized to an array chip containing >2,000 miRNA probes. b) Confirmation of array results by real-time PCR analysis for selected miRNA using the same RNA samples (from top to bottom: SEQ ID NO: 2, 14, 73, 157, 158, 159) used for the array study. The Ct value was presented as average and standard derivation from quadruplicate experiments. hsa-miR-130a-3p was selected as a positive control because, based on miRNA array and qRT-PCR assays, it was expressed at high levels in all 293 and CD34+ cell samples. The Ct value correlates inversely with the RNA concentration. n.d.—not detectable

FIG. 2. Analysis of miRNA regulated transgene expression. a) Schematic of Ad5/35 vectors used to test miRNA regulated expression. Description is in the text. The 3′ end of the GFP gene is linked to the 3′ untranslated region (UTR) of the globin gene. miRNA target sites were inserted into the 3 UTR. The GFP mRNA transcribed from the EF1a promoter therefore contains miRNA target sites. In contrast, mCherry™ expression is not regulated by the selected miRNAs. b) Transgene expression in 293-Cre cells. Cells were infected at the indicated MOIs with the Ad5/35 vector that lacks miRNA target sites (no miR) and the vectors containing the miRNA target sites. Shown is the GFP fluorescence intensity divided by the mCherry™ fluorescence intensity measured by flow cytometry at 48 h after infection. N=3. P values were calculated using unpaired t-test with unequal variance (GraphPad Prism 5 software). The p values for “no miR” vs “miR218-183” are 0.12; 0.0012; 0.02; and 0.0016 for MOIs 2, 5, 10, and 20 pfu/ cell, respectively. Note that the two promoters (PGK and Ef1a) are differently regulated and require different transcription factors. For the vector without miR target sites, with increasing MOIs, i.e. transgene copy numbers, GFP levels increase to a much greater degree than mCherry™ levels. c) Flow cytometry of transduced CD34+ cells 48 h post infection. Shown is the GFP/mCherry™ MF1ratio. N=3. The transduction studies in 293 and CD34+ cells were performed with first-generation vectors. The titers are given in plaque-forming units (Pfu). One pfu corresponds to 20 viral particles (vp).

FIG. 3. Transduction studies with HD-Ad5/35.ZFNmiR. a) Vector genome structure. The two ZFN subunits are linked through a self-cleaving viral 2A peptide. The ZFN coding sequence is upstream of miR-183/218 target sites and 3′UTR. Both ZFN subunits are transcribed from the EF1a promoter. In CD34+ cells, the mRNA will not be degraded and a polyprotein will be expressed which will subsequently be cleaved into the two ZFN subunits at the 2A peptide. b and d) Expression of ZFN protein in MO7e cells (b) or CD34+ cells (d) after transduction with the HD-Ad5/35.ZFNmiR vector (HD-ZFN) at the indicated MOIs. Cells were harvested 48 hours later and cell lysates were analyzed by Western blot with antibodies against the FokI domain. Actin B is used as loading control. c and a) T7E1 nuclease assay. Genomic DNA from transduced MO7e cells (c) or CD34+ cells (c) was subjected to a PCR assay based on a T7E1 nuclease that detects mutations [11]. PCR products were separated by PAGE electrophoresis. Bands that correspond to disrupted ccr5 alleles are marked by arrows. The expected size of cleavage products is 141 bp and 124 bp. The numbers below the lanes indicate the % of disrupted ccr5 alleles. Studies were done with CD34+ cells from donor A.

FIG. 4. Analysis of CD34+ cytotoxicity associated with HD-ZFN transduction. Studies were performed with CD34+ cells from donor A (a) and donor B (b) Shown is the percentage of Annexin V-positive cells at day 4 after transduction with an HD-Ad5/35 control vector containing the b-globin LCR (HD-bGlob) or the HD-ZFN vector at the indicated MOIs. Annexin V and 7AAD expression was analyzed by flow cytometry N=3. c) Cytotoxicity after infection of CD34+ cells with first generation (FG-ZFN) and helper-dependent (HD-ZFN) Ad5/35 vectors expressing the CCR5 ZFN. CD34+ cells from donor B were used. N=3. HD-ZFN vs FG-ZFN (MOI 1000): p=1.51×10⁻⁶, HD-ZFN vs FG-ZFN (MOI 10,000): p=2.83×10⁻⁸.

FIG. 5. Analysis of LTC-IC. CD34+ cells were transduced with HD-bGlob and HD-ZFN at the indicated MOIs. Three days later, cells were transferred to LTC-IC medium and cultured for 5 weeks. A total of 3,000 LT-CIC cells were then plated in methylcellulose supplemented with growth factors and cytokines. Two weeks later colonies were counted. Cells from all colonies per plate were combined and genomic DNA was isolated and subjected to T7E1 nuclease assay. a and b) Numbers of colonies per plate for donor A and B respectively. There was no difference in the ratio of BFU-E and CFU-GM colonies in the different groups. N=3 plates, n.s. non-significant (p>0.05), **p<0.05 c) Number of CFU from donor B cells transduced with FG-ZFN and HD-ZFN. d) T7E1 nuclease assay. CD34+ cells from donor A were used for transduction with HD-bGlob and HD-ZFN at an MOI of 5000 vp/cell. Genomic DNA was from colonies was isolated and subjected to T7E1 assay. A representative T7E1 nuclease assay of CFU/LTC-IC samples is shown.

FIG. 6. ccr5 gene knockout in NOD/SCID repopulating cells. a) Study design. Cryo-conserved CD34+ cells from donor A were cultured overnight under low cytokine concentration conditions and transduced with HD-bGlob or HD-ZFN at an MOI of 5,000 vp/cell for 24 hours. Cells were then washed and transplanted into sub-lethally irradiated NOG mice. Six weeks later, animals were euthanized and bone marrow cells, splenocytes and PBMC were collected. The percentage of human cells in collected cells was measured by flow cytometry for the pan-leukocyte marker CD45. Human donor cells were purified by magnetic-activated cell sorting (MACS) using beads conjugated with anti-human CD45 antibodies. CD45+ cells were used for the T7E1 nuclease assay. b) Engraftment rate based on the percentage of human CD45+ cells in total cells from bone marrow, spleen, and PBMCs. N=3. c) Number of colonies from MACS isolated human CD34+ cells in the bone marrow of transplanted mice. N=3. The difference between “no Ad” and “HD-ZFN” is not significant (p=0.061) d) Analysis of ccr5 gene disruption in human CD45+ cells from bone marrow of transplanted mice.

FIG. 7. Structure and functional analysis of an HD-Ad5/35 vector expressing a globin LCR specific TALEN. a) Target site of TALEN. Shown is the structure of the globin LCR with DNase hypersensitivity sites HS1 to HS5. The lower panel shows the 5′ sequence of the HS2 target site labeled by a horizontal arrow (SEQ ID NOs: 160 and 161). The lines above and below the sequence indicate the binding sites of the two TALEN subunits respectively. The vertical bold arrow marks the TALEN cleavage site. b) Structure of the HD-Ad5/35.TALENmiR (HD-TALEN) genome. In analogy to the ZFN vector, the two TALEN subunits were linked through a 2A peptide at the 3′ end to the miR183/218 target sequence-containing 3′ UTR. The N-terminus of TALEN (1) contained an influenza hemagglutinine (HA) tag. c) Expression of TALEN in MO7e cells. Cells were infected at an MOI of 1000 vp/cell and cell lysates were analyzed by Western blot with antibodies specific for HA-tag, d) T7E1 nuclease assay analysis. Genomic DNA was isolated from MO7e cells 48 hours after infection at an MOI of 10³, 2×10³ vp/cell and subjected to PCR using globin LCR H2 specific primers. The expected length of PCR products is 608, 434, 174 bp.

FIG. 8. Flow chart of a non-limiting and exemplary hematopoietic stem cell mobilization and treatment schedule.

FIG. 9. In vitro transduction studies with Ad5/35 vectors containing long or short fiber shafts. Ad5/355 and Ad5/35L contain a CMV-luciferase cassette. A) Ability to use factor X to transduce CHO-K1 cells expressing HSPG (left panel) or CHO-E606 cells that lack HSPG expression (99) (right panel). Factor X enhanced transduction requires a long fiber shaft and HSPGs. The MOI used was 50 pfu/cell. FX concentration was 7.5 μg/ml. N=3. B) Transduction of human CD34+ cells at different MOIs. N=3.

FIG. 10. Ad5/35++ in vivo transduction of HSCs after mobilization: A) HCSs were mobilized in huCD46tg mice by s.c. injections of human recombinant G-CSF (5 μg/mouse/day, 4 days) followed by an s.c. injection of AMD3100 (5 mg/kg) eighteen hours after the last G-CSF injection. A total of 2×10⁹ pfu of Ad5/35++GFP was injected i.v. one hour after AMD-3100. B) Transduction was analyzed by harvesting PBMCs six and 72 hour after Ad injection and culturing them for 2 days to allow for transgene expression. Shown is the percentage of GFP-positive LSK cells in peripheral blood. N=5 C) Transduction was analyzed in mobilized and non-mobilized animals by harvesting bone marrow and spleen at day 3, 7 and 14 after Ad injection. Shown is the percentage of GFP-positive LSK cells in the bone marrow and spleen. N=5. In vivo transduction of LSK cells was inefficient without mobilization. Notably, intravenous injection of Ad5/35 vector does not cause liver toxicity in mice and non-human primates.

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

As used herein, the amino acid residues are abbreviated as follows: alanine (Ala, A), asparagine (Asn; N), aspartic acid (Asp; D), arginine (Arg; R), cysteine (Cys; C), glutamic acid (Glu; E), glutamine (Gln; Q), glycine (Gly; histidine (His; H), isoleucine (Ile; I), leucine (Leu; L), lysine (Lys; K), methionine (Met; M), phenylalanine (Phe; F), proline (Pro; P), serine (Sec; S), threonine (Thr; T), tryptophan (Trp; W), tyrosine (Tyr; Y), and valine (Val; V).

All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively, Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In a first aspect, the invention provides recombinant nucleic acid expression cassette, comprising (a) at least one first nucleic acid module comprising
(i) first coding region encoding a nuclease capable of generating a DNA break in a CD34+ cell genomic target of interest; and
(ii) a second coding region encoding one or more miRNA target sites located in a 3′ untranslated region of the first coding region and at least 60 nucleotides downstream of a translation al stop codon of the first coding region, wherein miRNAs that bind to the one or more encoded miRNA target sites are highly expressed in virus producer cells but not expressed, or expressed at low levels, in CD34+ cells,
wherein the first nucleic acid module is operatively linked to a promoter that is active in in CD34+ cells.

As shown in the examples that follow, the expression cassettes of the invention can be used as to produce the genome of helper dependent adenoviruses of the invention, which can in turn used for significantly improved methods of in vivo gene engineering in CD34+ cells, such as hematopoietic cells. For example, the cassette can be used for cloning into a vector (such as a plasmid) containing other necessary components for helper-dependent Ad viral production.

In one embodiment, the cassette or vector derived therefrom further comprises a second nucleic acid module encoding a CD46 binding adenoviral fiber polypeptide. In a further embodiment, the cassette or vector derived therefrom further comprises an inverted terminal repeat (ITR) at each terminus of the recombinant nucleic acid vector, wherein the ITR derived from a CD46-binding adenovirus serotype. In a further embodiment, the cassette or vector derived therefrom further comprises a packaging signal from a CD46-binding adenovirus serotype.

Adenoviral (Ad) genomes of the invention have a large capacity (˜30kb) that can accommodate large payloads, including several nuclease expression cassettes and homologous donor template, which can be used for transducing CD34+ cells in vivo. During Ad amplification in producer cells, massive amounts of nuclease will be produced, if it is not suppressed. High levels of nuclease expression is poorly tolerated in Ad producer cells, which prevents the rescue of vectors or selects for recombined vector genomes and deletion of EN expression cassettes.

The production of the helper dependent adenoviruses is greatly enhanced by suppressing expression of the nuclease in HD-adenoviral producer cells, which is accomplished in the present invention via a miRNA-based system for regulation of gene expression based on miRNA expression profiling of producer cells vs CD34+ cells. Specifically, target sites for miRNA that are highly expressed in virus producer cells but not expressed, or expressed at low levels, in CD34+ cells are transcribed from the cassette as a fusion linked to the nuclease mRNA. When expressed in HD-producer cells, the miRNAs bind to the mRNA target site and lead to degradation of the nuclease-mRNA target site hybrid, thus reducing or eliminating expression of the nuclease in the producer cells and greatly facilitating (in combination with helper Ad virus) production of the recombinant HD-adenoviruses of the invention without vector genomic rearrangement. As CD34+ cells have no or a much reduced amount of miRNA is available for binding to the miRNA target sites, expression of the nuclease protein occurs, permitting effective gene editing.

As used herein, a “producer cell” is any cell type that can be used for production of high titers of adenovirus. It is well within the level of skill in the art to determine an appropriate producer cell. In one embodiment, the producer cells are suitable for production of helper-dependent adenovirus. Non-limiting examples of producer cells for use in the invention include, but are not limited to human embryonic kidney (HEK) 293 cells, HEK 293-Cre cells, PerC6 cells, HCT 116 cells, etc. In one embodiment, the producer cells are HEK 293 cells or HEK 293-Cre cells.

As used herein, CD34+ cells are cells that express the CD34 protein as a cell surface protein. Exemplary CD34+ cells are hematopoietic progenitor cells (such as hematopoietic stem cells (HSC)) and progenitor/adult stem cells of other lineages (i.e., mesenchymal stem cells, endothelial progenitor cells, mast cells, dendritic cells, etc.) In one embodiment, the CD34+ cells are hematopoietic progenitor cells, such as HSC.

As used herein, a miRNA is “highly expressed” in the producer cell if it as a real time qRT-PCT Ct value less than 35. A miRNA is expressed at low levels if it has a real time qRT-PCT Ct value greater than 39. As is understood by those of skill in the art, in a real time PCR assay a positive reaction is detected by accumulation of a fluorescent signal. The Ct (cycle threshold) is defined as the number of cycles required for the fluorescent signal to cross the threshold (i.e., exceeds background level). Ct levels are inversely proportional to the amount of target nucleic acid in the sample (i.e., the lower the Ct level the greater the amount of target nucleic acid in the sample). Cts of 39 or more are weak reactions indicative of minimal amounts of target nucleic acid which could represent an infection state or environmental contamination.

Any suitable technique can be used to identify miRNA that are highly expressed in a producer cell of interest and not expressed or expressed at low levels in CD34+ cells of interest, including but not limited to the methods described in the examples that follow.

Exemplary miRNAs that are highly expressed in HEK-293 and HEK-293-Cre cells and not in CD34+ hematopoietic cells include, but are not limited to RNA sequences comprising:
(a) CACUGGUAGA (SEQ ID NO: 1) (has-miR183-5p core)
(b) UGUGCUUGAUCUAA (SEQ ID NO: 2) (has-miR218-5p core); and
(c) CACUAGCACA (SEQ ID NO: 3) (miR96-5p core).

As shown in the examples that follow, expression cassettes encoding a target site for a miRNA comprising one or more of these miRNAs are effective in suppressing nuclease expression in producer cells. As will be understood by one of skill in the art, such target sites comprise a reverse complement of the miRNA to be targeted. In non-limiting examples:
The miRNA to be targeted is 5′ CACUGGUAGA 3′ (SEQ ID NO: 1) (has-miR-183-5p core); the reverse complement target site comprises/consists of 5′UCUACCAGUG 3′ (SEQ ID NO: 4);
The miRNA to be targeted is 5′ CACUAGCACA 3′ (SEQ ID NO: 3) (miR-96-5p core); the reverse complement target site comprises/consists of 5′ UGUGCUAGUG 3′ (SEQ ID NO: 5);
The miRNA to be targeted is 5′ UGUGCUUGAUCUAA 3′ (SEQ ID NO: 2) (has-miR-218-5p core); the reverse complement target site comprises/consists of 5′ UUAGAUCAAGCACA 3′ (SEQ ID NO: 6);
The miRNA to be targeted is 5′UAUGGCACUGGUAGAAUUCACU 3′(SEQ ID NO: 14) (has-miR-183-5p); the reverse complement target site comprises/consists of 5′AGUGAAUUCUACCAGUGCCAUA 3′(SEQ ID NO: 7);
The miRNA to be targeted is 5′ UUUGGCACUAGCACAUUUUUGCU 3′ (SEQ ID NO: 73) (miR-96-5p); the reverse complement target site comprises/consists of 5′ AGCAAAAAUGUGCUAGUGCCAAA 3′(SEQ ID NO: 8);
The miRNA to be targeted is 5′UUGUGCUUGAUCUAACCAUGU 3′ (SEQ ID NO: 48) (has-miR-218-5p); the reverse complement target site comprises/consists of 5′ AGAUGGUUAGAUCAAGCACAA 3′ (SEQ ID NO: 9).

As will be understood by those of skill in the art, the miRNAs may be present in producer cells in various processed versions, each containing the core sequence noted above. Thus, in various further embodiments, a target site comprises or consists of a reverse complement of one or more of the following (all in a 5′ to 3′ orientation), or combinations thereof:

miR-hsa-183-5p processing
(SEQ ID NO: 10)
UGUAUGGCACUGGUAGAAUU
(SEQ ID NO: 11)
UGUAUGGCACUGGUAGAAUUCA
(SEQ ID NO: 12)
UGUAUGGCACUGGUAGAAUUCACU
(SEQ ID NO: 13)
GUAUGGCACUGGUAGAAUUCACU
(SEQ ID NO: 14)
UAUGGCACUGGUAGAAUUCACU
(SEQ ID NO: 15)
UAUGGCACUGGUAGAAUUCACUG
(SEQ ID NO: 16)
UAUGGCACUGGUAGAAUUCA
(SEQ ID NO: 17)
UAUGGCACUGGUAGAAUUCAC
(SEQ ID NO: 18)
UAUGGCACUGGUAGAAUUC
(SEQ ID NO: 19)
UAUGGCACUGGUAGAAUUCACUGU
(SEQ ID NO: 20)
UAUGGCACUGGUAGAAUU
(SEQ ID NO: 21)
UAUGGCACUGGUAGAAU
(SEQ ID NO: 22)
UAUGGCACUGGUAGAA
(SEQ ID NO: 23)
UAUGGCACUGGUAGA
(SEQ ID NO: 24)
AUGGCACUGGUAGAAUUCACU
(SEQ ID NO: 25)
AUGGCACUGGUAGAAUUCACUG
(SEQ ID NO: 26)
AUGGCACUGGUAGAAUUCA
(SEQ ID NO: 27)
AUGGCACUGGUAGAAUUCACUGU
(SEQ ID NO: 28)
AUGGCACUGGUAGAAUUCAC
(SEQ ID NO: 29)
AUGGCACUGGUAGAA
(SEQ ID NO: 30)
AUGGCACUGGUAGAAUU
(SEQ ID NO: 31)
AUGGCACUGGUAGAAUUC
(SEQ ID NO: 32)
AUGGCACUGGUAGAAU
(SEQ ID NO: 33)
UGGCACUGGUAGAAUUCACUG
(SEQ ID NO: 34)
UGGCACUGGUAGAAUUCAC
(SEQ ID NO: 35)
CACUGGUAGAAUUCACUG
(SEQ ID NO: 36)
CACUGGUAGAAUUCA
(SEQ ID NO: 37)
CACUGGUAGAAUUCAC
(SEQ ID NO: 38)
CACUGGUAGAAUUCACU
(SEQ ID NO: 39)
ACUGGUAGAAUUCACU
mir-hsa-218-5p processing
(SEQ ID NO: 40)
GUUGUGCUUGAUCUAACCAUGU
(SEQ ID NO: 41)
GUUGUGCUUGAUCUAACCAU
(SEQ ID NO: 42)
UUGUGCUUGAUCUAACCAU
(SEQ ID NO: 43)
UUGUGCUUGAUCUAACCAUGUGGU
(SEQ ID NO: 44)
UUGUGCUUGAUCUAACCAUGUGGU
(SEQ ID NO: 45)
UUGUGCUUGAUCUAACCA
(SEQ ID NO: 46)
UUGUGCUUGAUCUAACCAUGUGG
(SEQ ID NO: 47)
UUGUGCUUGAUCUAAC
(SEQ ID NO: 48)
UUGUGCUUGAUCUAACCAUGU
(SEQ ID NO: 49)
UUGUGCUUGAUCUAACC
(SEQ ID NO: 50)
UUGUGCUUGAUCUAACCAUGUG
(SEQ ID NO: 51)
UUGUGCUUGAUCUAA
(SEQ ID NO: 52)
UGUGCUUGAUCUAACCAUGU
(SEQ ID NO: 53)
UGUGCUUGAUCUAACCAUGUG
(SEQ ID NO: 54)
UGUGCUUGAUCUAACCAUGUGGU
(SEQ ID NO: 55)
GUGCUUGAUCUAACCAUGU
(SEQ ID NO: 56)
GUGCUUGAUCUAACCAUGUG
(SEQ ID NO: 57)
UGCUUGAUCUAACCAUGUG
(SEQ ID NO: 58)
UGCUUGAUCUAACCAUGU
(SEQ ID NO: 59)
GCUUGAUCUAACCAUGU
(SEQ ID NO: 60)
GCUUGAUCUAACCAUG
(SEQ ID NO: 61)
GCUUGAUCUAACCAU
(SEQ ID NO: 62)
GCUUGAUCUAACCAUGUGGU
(SEQ ID NO: 63)
GCUUGAUCUAACCAUGUG
(SEQ ID NO: 64)
CUUGAUCUAACCAUGU
(SEQ ID NO: 65)
CUUGAUCUAACCAUGUG
(SEQ ID NO: 66)
CUUGAUCUAACCAUG
(SEQ ID NO: 67)
UUGAUCUAACCAUGU
(SEQ ID NO: 68)
UUGAUCUAACCAUGUG
(SEQ ID NO: 69)
UUGAUCUAACCAUGUGGU
(SEQ ID NO: 70)
UUGAUCUAACCAUGUGGUU
(SEQ ID NO: 71)
UUGAUCUAACCAUGUGG
miR-96-5p processing;
(SEQ ID NO: 72)
UUUUGGCACUAGCACAUUUUUGCU
(SEQ ID NO: 73)
UUUGGCACUAGCACAUUUUUGCU
(SEQ ID NO: 74)
UUUGGCACUAGCACAUUUUUG
(SEQ ID NO: 75)
UUUGGCACUAGCACAUUUUU
(SEQ ID NO: 76)
UUUGGCACUAGCACAUUUUUGC
(SEQ ID NO: 77)
UUUGGCACUAGCACAUUUU
(SEQ ID NO: 78)
UUUGGCACUAGCACAUUU
(SEQ ID NO: 79)
UUUGGCACUAGCACA
(SEQ ID NO: 80)
UUUGGCACUAGCACAUU
(SEQ ID NO: 81)
UUUGGCACUAGCACAUUUUUGCUU
(SEQ ID NO: 82)
UUUGGCACUAGCACAU
(SEQ ID NO: 83)
UUGGCACUAGCACAUUUUUGC
(SEQ ID NO: 84)
UUGGCACUAGCACAUUUUUGCU
(SEQ ID NO: 85)
GGCACUAGCACAUUUUUGCU
(SEQ ID NO: 86)
CACUAGCACAUUUUUGCU
(SEQ ID NO: 87)
CACUAGCACAUUUUUGC
(SEQ ID NO: 88)
ACUAGCACAUUUUUG
(SEQ ID NO: 89)
CUAGCACAUUUUUGCU
(SEQ ID NO: 90)
CUAGCACAUUUUUGC.

The second coding region may encode one or more miRNA target sites. Thus, in various embodiments, the second coding region encodes 1, 2, 3, 4, 5, 6, or more miRNA target sites (i.e.: reverse complements of a miRNA of interest). Each encoded target site may be the same or different. For example, all target sites may be reverse complements of the same miRNA or different processed forms of the same miRNA. In another non-limiting example, the second coding region may include target sites for different miRNAs; for example, one or more target sites for miR-hsa-183 miRNA core-containing miRNAs, and one or more target sites for the miR-hsa-218-5p core-containing miRNAs. The presence of target sites of different miRNAs can maximize the inhibitory activity miRNAs as long as there is appropriate copy number of that miRNA in the cell. When more than one target site is encoded in the second coding region, the target sites may be directly adjacent or may be separated by a spacer of a variable number of nucleotides. In various non-limiting examples, the spacer may be between 1-10, 2-9, 3-8, 4-7, or 5-6 nucleotides in length. Such spacer regions may provide useful DNA flexibility; it is well within the level of skill in the art to determine an appropriate number of spacer residues between encoded target sites based on the disclosure herein. In various further non-limiting embodiments, the second coding sequence may comprise or consist of a sequence selected from the group consisting of SEQ ID NO: 142 (miR-183 target sites), SEQ ID NO: 143 (miR-218 target sites), and SEQ ID NO: 144 (miR-183/218 target sites):

In all embodiments, the second coding region is located within a 3′ untranslated region of the first coding region, at least 60 nucleotides downstream of a translational stop codon of the first coding region, to maximize efficacy of mRNA degradation upon miRNA binding to the target site(s) after transcription of the fused first and second coding regions. The second coding region may be placed with a region of the 3′UTR that is less prone to secondary structure formation (i.e.: an AT-rich region).

The first coding region encodes a nuclease capable of generating a DNA break in a CD34+ cell genomic target of interest; such a DNA break may be a single stranded or a double stranded break. There are a number of different site-specific endonculeases EN platforms to generate site-specific DNA breaks in the genome. One group of ENs contains DNA binding protein domains. This group includes meganucleases with DNA binding and nuclease properties as well as zinc-finger nucleases (ZFNs) and transcription activator-like effector nucleases (TALENs) in which the DNA binding domain is fused with the bacterial endonuclease FokI. Because DNA cleavage by Fold requires two FokI molecules bound to each of the DNA strands, two subunits of the FokI containing ENs have to be expressed; in this embodiment, the two nuclease subunits may be linked through a cleavable peptide. A second group of ENs is based on RNA-guided DNA recognition and utilizes the clustered regularly interspaced short palindromic repeats (CRISPR)/Cas9 bacterial system. Thus, it is well within the level of skill in the art to design a site specific EN capable of generating a DNA break in a CD34+ cell genomic target of interest. Non-limiting examples are provided in the examples that follow.

In one non-limiting embodiment, the first coding region encodes a ZFN that targets the human Chemokine Receptor Type 5 (CCR5) gene, where the first coding sequence comprises or consists of the following sequence:

hCCR5-ZFN
(SEQ ID NO: 91)
MDYKDHDGDYKDHDIDYKDDDDKMAPKKKRKVGIHGVPAAMAERPFQCRI
CMRNFSDRSNLSRHIRTHTGEKPFACDICGRKFAISSNLNSHTKIHTGSQ
KPFQCRICMRNFSRSDNLARHIRTHTGEKPFACDICGRKFATSGNLTRHT
KIHLRGSQLVKSELEEKKSELRHKLKYVPHEYIELIEIARNSTQDRILEM
KVMEFFMKVYGYRGKHLGGSRKPDGAIYTVGSPIDYGVIVDTKAYSGGYN
LPIGQADEMERYVEENQTRNKHLNPNEWWKVYPSSVTEFKFLFVSGHFKG
NYKAQLTRLNHITNCNGAVLSVEELLIGGEMIKAGTLTLEEVRRKFNNGE
INFRSGSGEGRGSLLTCGDVEENPGPRMDYKDHDGDYKDHDIDYKDDDKD
MAPKKKRKVGIHGVPAAMAERPFQCRICMRNFSRSDNLSVHIRTHTGEKP
FACDICGRKFAQKINLQVHTKIHTGEKPFQCRISMRNFSRSDVLSEHIRT
HTGEKPFACDICGRFGAQRNHRTTHTKIHLRGSQLVKSELEEKKSELRHK
LKYVPHEYIELIEIARNSTQDRILEMKVMEFFMKVYGYRGKHLGGSRKPD
GAIYTVGSPIDYGVTVDTKAYSGGYNLPIGQADEMQRYVKENQTRNKHIN
PNEWWKVYPSSVTEFKFLFVSGHFKGNYKAQLTRLNHKTNCNGAVLSVEE
LLIGGEMIKAGTLTLEEVRRKFNNGEINF

In another non-limiting embodiment, the first coding region encodes a ZFN that targets the human β-globin gene, where the first coding sequence comprises or consists of the following sequence:

TALEN globin
(SEQ ID NO: 92)
MVYPYDVPDYAELPPKKKRKVGIRIQDLRTLGYSQQQQEKIKPKVRSTVA
QHHEALVGHGFTHAHIVALSQHPAALGTVAVKYQDMIAALPEATHEAIVG
VGKQWSGARALEALLTVAGELRGPPLQLDTGQLLKIAKRGGVTAVEAVHA
WRNALTGAPLTPAQVVAIASNIGGKQALETVQRLLPVLCQDHGLTPDQVV
AIASNIGGKQALETVQRLLPVLCQDHGLTPDQVVAIASNNGGKQALETVQ
RLLPVLCQAHGLTPAQVVAIASNIGGKQALETVQRLLPVLCQDHGLTPAQ
VVAIASNGGGKQALETVQRLLPVLCQDHGLTPDQVVAIASNIGGKQALET
VQRLLPVLCQDHGLTPAQVVAIASHDGGKQALETVQRLLPVLCQDHGLTP
DQVVAIASNIGGKQALETVQRLLPVLCQDHGLTPAQVVAIASNGGGKQAL
ETVQRLLPVLCQAHGLTPAQVVAIASNNGGKQALETVQRLLPVLCQDHGL
TPEQVVAIASNGGGKQALETVQRLLPVLCQAHGLTPAQVVAIASNGGGKQ
ALETVQRLLPVLCQDHGLTPEQVVAIASNGGGKQALETVQRLLPVLCQAH
GLTPDQVVAIASNGGGKQALETVQRLLPVLCQDHGLTPDQVVAIASNIGG
KQALETVQRLLPVLCQDHGLTPDQVVAIASNGGGKQALETVQRLLPVLCQ
DHGLTPEQVVAIASNGGGKQALETVQRLLPVLCQAHGLTPDQVVAIASHD
GGKQALETVQRLLPVLCQAHGLTPEQVVAIASNGGGKQALESIVAQLSRP
DPALAALLVKSELEEKKSELRHKLKYVPHEYIELIEIARNPTQDRILEMK
VMEFFMKVYGYRGEHLGGSRKPDGAIYTVGSPIDYGVIVDTKAYSGGYNL
PIGQADAMQSYVEENQTRNKHINPNEWWKVYPSSVTEFKFLFVSGHFKGN
YKAQLTRLNHITNCNGAVLSVEELLIGGEMIKAGTLTLEEVRRKFNNGEI
NFLDGSGEGRGSLLTCGDVEENPGPVYPYDVPDYAELPPKKKRKVGIRIQ
DLRTLGYSQQOQEKIKPKVRSTVAQHHEALVGHGFTHAHIVALSQHPAAL
GTVAVKYQDMIAALPEATHEAIVGVGKQWSGARALEALLTVAGELRGPPL
QDTGQLLKIAKRGGVTAVEAVHAWRNALTGAPLTPDQVVAIASNIGGKQA
LETVQRLLPVLCQAHGLTPAQVVAIASNIGGKQALETVQRLLPVLCQDHG
LTPAQVVAIASNIGGKQALETVQRLLPVLCQAHGLTPDQVVAIASHDGGK
QALETVQRLLPVLCQAHGLTPDQVVAIASNIGGKQALETVQRLLPVLCQA
HGLTPAQVVAIASHDGGKQALETVQRLLPVLCQAHGLTPDQVVAIASNGG
GKQALETVQRLLPVLCQAHGLTPDQVVAIASNIGGKQALETVQRLLPVLC
QDHGLTPDQVVAIASNGGGKQALETVQRLLPVLCQAHGLTPDQVVAIASN
GGGKQALETVQRLLPVLCQAHGLTPAQVVAIASNGGGKQALETVQRLLPV
LCQAHGLTPEQVVAIASNGGGKQALETVQRLLPVLCQAHGLTPEQVVAIA
SHDGGKQALETVQRLLPVLCQAHGLTPAQVVAIASNIGGKQALETVQRLL
PVLCQAHGLTPAQVVAIASNNGGKQALETVQRLLPVLCQDHGLTPAQVVA
IASNGGGKQALETVQRLLPVLCQAHGLTPDQVVAIASNNGGKQALETVQR
LLPVLCQAHGLTPAQVVAIASHDGGKQALETVQRLLPVLCQAHGLTPEQV
VAIASNGGGKQALESIVAQLSRPDPALAALLVKSELEEKKSELRHKLKYV
PHEYIELIEIARNPTQDRILEMKVMEFFMKVYGYRGEHLGGSRKPDGAIY
TVGSPIDYGVIVDTKAYSGGYNLPIGQAREMQRYVEENQTRNKHINPNNE
WWKVYPSSVTEFKFLFVSGHFKGNYKAQLTRLNHITNCNGAVLSVEELLI
GGEMIKAGTLTLEEVRRKFNNGEINFLD

In a further non-limiting embodiment, the first coding region encodes a ZFN that targets the monkey Chemokine Receptor Type 5 (CCR5) gene, where the first coding sequence comprises or consists of the following sequence:

Monkey ZIFN-CCR5
(SEQ ID NO: 93)
MDYKDHDGDYKDHDIDYKDDDDKMAPKKKRKVGIHGVPAAMAERPFQCRI
CMRNFSRSDNLSVHIRTHTGEKPFACDICGRKFAANHHRINHTKIHTGSQ
KPFQCRICMRNFSDRSDLSRHIRTHTGEKPFACDICGRKFARSDHLSRHT
KIHTGSQKPFQCRICMRNFSQSGNLARHIRTHTGEKPFACDICGRKFAQR
NDRKSHTKIHLRGSQLVKSELEEKKSELRHKLKYVPHEYIELIEIARNST
QDRILEMKVMEFFMKVYGYRGKHLGGSRKPDGAIYTVGSPIDYGVIVDTK
AYSGGYNLPIGQADEMERYVEENQTRDKHLNPNEWWKVYPSSVTEFKFLF
VSGHFKGNYKAQLTRLNHITNCNGAVLSVEELLIGGEMIKAGTLTLEEVR
RKFNNGEINFRSGSGEGRGSLLTCGDVEENPGPRMDYKDHDGDYKDHDID
YKDDDDKMAPKKKRKVGIHGVPAAMAERPFQCRICMRNFSRSDHLSQHIR
THTGEKPFACDICGRKFATSANRTTHTKIHTGSQKPFQCRICMRNFSERG
TLARHIRTHTGEKPFACDICGRKFAQSSDLRRHTKIHTGSQKPFQCRICM
RNFSQSSDLSRHIRTHTGEKPFACDICGRKFACRSNLKKHTKIHLRGSQL
VKSELEEKKSELRHKLKYVPHEYIELIEIARNSTQDRILEMKVMEFFMKV
YGYRGKHLGGSRKPDGAIYTVGSPIDYGVIVDTKAYSGGYNLPIGQADEM
QRYVKENQTRNKHINPNEWWKVYPSSVTEFKFLFVSGHFKGNYKAQLTRL
NRKTNCNGAVLSVEELLIGGEMIKAGTLTLEEVRRKFNNGEINF

As will be understood by those of skill in the art, the site-specific EN designed my target any CD34÷ genomic target of interest. In various non-limiting embodiments, the nuclease is capable of generating a DNA break in a CD34+ cell genomic target selected from the group consisting of genes encoding CCR5, β-globin, Complement receptor 2 (CR2) (Epstein Ban Virus (EBV) receptor), Niemann-Pick disease, type C1 receptor (NPC1) Ebola receptor), angiotensin-converting enzyme 2 receptor (ACE2) SARS receptor), and genes that encode proteins that can lead to lysosomal storage disease if misfolded.

In another embodiment, the first coding region may encode a nuclease that has been modified to permit shortened expression in vivo. In one embodiment, the first coding region encodes a fusion of the nuclease and a PEST peptide, i.e. a peptide sequence that is rich in proline, glutamic acid, serine, and threonine, which serves as a signal peptide for protein degradation. In one embodiment, a sequence encoding the PEST amino acid sequence of ornithine decarboxylase (mODC) (Residues 422-461) can be used (FPPEVEEQDDGTLPMSCAQEGMDR) (SEQ ID NO: 102), such as at the N-terminus of any embodiments of the nuclease disclosed herein.

In a further embodiment, the first coding region encodes a fusion of the nuclease and the FRB* domain (SEQ ID NO: 106), such as at the N-terminus of any embodiments of the nuclease disclosed herein.

Rapamycin binds to FKBP12 to form a complex that inhibits the FKBP12-rapamycin-associated protein (FRAP). The minimal region within FRAP sufficient for FKBP12-rapamycin binding is an 89 amino acid domain termed FRB (FKBP-rapamycin binding). A mutated form of FRB with a T2098L, substitution (FRB*) causes the degradation of fusion proteins. Upon recruitment of FKBP12 using rapamycin, the fusion protein is thermodynamically stabilized, and activity of the target protein is recovered. Thus, the period of nuclease expression can be controlled.

In another embodiment, a TALEN DNA recognition sequence can be fused in-frame to the N-terminus of a TALEN ORF. When the nuclease is expressed in CD34+ cells, it will cleave its own gene inside the vector thereby inactivating the nuclease. This will not occur during HD-Ad production because TALEN expression is suppressed in 293 cells through miRNA regulation). Such a sequence is shown below:

(SEQ ID NO: 103)
MGHPHPDKLQKGGGSGGGSGGGSDYKDHDGDYKDHDIDYKDDDDKMAPK
KKRKVGIHGVPAAMAERPFQCRICMRNFSDRSNLSRHIRTHTGEKPFACD
ICGRKFAISSNLNSHTKIHTGSQKPFQCRICMRNFSRSDNLARHIRTHTG
EKPFACDICGRKFATSGNLTRHTKIHLRGSQLVKSELEEKKSELRHKLKY
VPHEYIELIEIARNSTQDRILEMKVMEFFMKVYGYRGKHLGGSRKPDGAI
YTVGSPIDYGVIVDTKAYSGGYNLPIGQADEMERYVEENQTRNKHLNPNE
WWKVYPSSVTEFKFLFVSGHFKGNYKAQLRTLNHITNCNGAVLSVEELLI
GGEMIKAGTLTLEEVRRKFNNGEINFRSGSGEGRGSLLTCGDVEENPGPR
MDYKDHDGDYKDHDIDYKDDDDKMAPKKKRKVGIHGVPAAMAERPFQCRI
CMRNFSRSDNLSVHIRTHTGEKPFACDICGRKFAQKINLQVHTKIHTGEK
PFQCRICMRNFSRSDVLSEHIRTHTGEKPFACDICGRKFAQRNHRTTHTK
IHLRGSQLVKSELEEKKSELRHKLKYVPHEYIELIEIARNSTQDRILEMK
VMEFFMKVYGYRGKHLGGSRKPDGAIYTVGSPIDYGVIVDTKAYSGGYNL
PIGQADEMQRYVKENQTRNKHINPNEWWKVYPSSVTEFKFLFVSGHFKGN
YKAQLTRLNHKTNCNGAVLSVEELLIGGEMIKAGTLTLEEVRRKFNNGEI
NF

Transcription of the first coding region and the second coding region result are controlled by a single promoter and results in a fusion RNA expression product. Thus, the first coding region and the second coding region may have a nucleic acid linker sequence of any suitable length between them, so long as the linker sequence does not contain a transcriptional stop polyadenylation signal.

As will be understood by those of skill in the art, the insert capacity of HD-Ad vectors is 30 kb which allows the accommodation of multiple first nucleic acid modules (and thus multiple first and second coding regions), which can be used, for example, to generate HD-Ad capable of simultaneous editing of multiple target genes in CD34+ cells for gene therapy purposes or to establish relevant models for multigenic human diseases.

Each of the first and second coding regions are operatively linked to a promoter that is active in CD34+ cells. As used herein, the term “operatively linked” refers to an arrangement of elements wherein the promoter function to permit expression of the first and second coding regions, regardless of the distance between the promoter the coding regions on the expression cassette. Any promoter that is active in CD34+ cells can be used. In various non-limiting embodiments, the promoter is selected from the group consisting of an EF1α promoter, a phosphoglycerate kinase (PGK) 1 promoter, and ubiquitin gene promoter. In one embodiment, the promoter is also active in the producer cells.

In various further embodiments, the promoter to drive expression of the first nucleic acid module comprises or consists or a nucleic acid sequence selected from the group consisting of the sequences shown below.

PGK
(SEQ ID NO: 145)
CACGGGGTTGGGGTTGCGCCTTTTCCAAGGCAGCCCTGGGTTTGCGCAGG
GACGCGGCTGCTCTGGGCGTGGTTCCGGGAAACGCAGCGGCGCCGACCCT
GGGTCTCGCACATTCTTCACGTCCGTTCGCAGCGTCACCCGGATCTTCGC
CGCTACCCTTGTGGGCCCCCCGGCGACGCTTCCTGCTCCGCCCCTAAGTC
GGGAAGGTTCCTTGCGGTTCGCGGCGTGCCGGACGTGACAAACGGAAGCC
GCACGTCTCACTAGTACCCTCGCAGACGGACAGCGCCAGGGAGCTGGCAG
CGCGCCGACCGCGATGGGCTGTGGCCAATAGCGGCTGCTCAGCGGGGCGC
GCCGAGAGCAGCGGCCGGGAAGGGGCGGTGCGGGAGGCGGGGTGTGGGGC
GGTAGTGTGGGCCCTGTTCCTGCCCGCGCGGTGTTCCGCATTCTGCAAGC
CTCCGGAGCGCACGTCGGCAGTCGGCTCCCTCGTTGACCGAATCACCGAC
CTCTCTCCCCA
EF1A
(SEQ ID NO: 146)
GAGTAATTCATACAAAAGGACTCGCCCCTGCCTTGGGGAATCCCAGGGAC
CGTCGTTAAACTCCCACTAACGTAGAACCCAGAGATCGCTGCGTTCCCGC
CCCCTCACCCGCCCGCTCTCGTCATCACTGAGGTGGAGAAGAGCATGCGT
GAGGCTCCGGTGCCCGTCAGTGGGCAGAGCGCACATCGCCCACAGTCCCC
GAGAAGTTGGGGGGAGGGGTCGGCAATTGAACCGGTGCCTAGAGAAGGTG
GCGCGGGGTAAACTGGGAAAGTGATGTCGTGTACTGGCTCCGCCTTTTTC
CCGAGGGTGGGGGAGAACCGTATATAAGTGCAGTAGTCGCCGTGAACGTT
CTTTTTCGCAACGGGTTTGCCGCCAGAACACAGGTAAGTGCCGTGTGTGG
TTCCCGCGGGCCTGGCCTCTTFACGGGTTATGGCCCTTGCGTGCCTTGAA
TTACTTCCACGCCCCTGGCTGCAGTACGTGATTCTTGATCCCGAGCTTCG
GGTTGGAAGTGGGTGGGAGAGTTCGAGGCCTTGCGCTTAAGGAGCCCCTT
CGCCTCGTGCTTGAGTTGAGGCCTGGCCTGGGCGCTGGGGCCGCCGCGTG
CGAATCTGGTGGCACCTTCGCGCCTGTCTCGCTGCTTTCGATAAGTCTCT
AGCCATTTAAAATTTTTGATGACCTGCTGCGACGCTTTTTTTCTGGCAAG
ATAGTCTTGTAAATGCGGGCCAAGATCTGCACACTGGTATTTCGGTTTTT
GGGGCCGCGGGCGGCGACGGGGCCCGTGCGTCCCAGCGCACATGTTCGGC
GAGGCGGGGCCTGCGAGCGCGGCCACCGAGAATCGGACGGGGGTAGTCTC
AAGCTCGCCGGCCTGCTCTGGTGCCTGGCCTCGCGCCGCCGTGTATCGCC
CCGCCCTGGGCGGCAAGGCTGGCCCGGTCGGCACCAGTTGCGTGAGCGGA
AAGATGGCCGCTTCCCGGCCCTGCTGCAGGGAGCTCAAAATGGAGGACGC
GGCGCTCGGGAGAGCGGGCGGGTGAGTCACCCACACAAAGGAAAAGGGCC
TTTCCGTCCTCAGCCGTCGCTTCATGTGACTCCACGGAGTACCGGGCGCC
GTCCAGGCACCTCGATTAGTTCTCGAGCTTTTGGAGTACGTCGTCTTTAG
GTTGGGGGGAGGGGTTTTATGCGATGGAGTFTCCCCACACTGAGTGGGTG
GAGACTGAAGTTAGGCCAGCTTGGCACTTGATGTAATTCTCCTTGGAATT
TGCCCTTTTTGAGTTTGGATCTTGGTTCATTCTCAAGCCTCAGACAGTGG
TTCAAAGTTTTTTTCTTCCATTTCAGGTGTCGTGA

Ubiquitin gene promoter:

(SEQ ID NO: 104)
AAGTTTCCAGAGCTTTCGAGGAAGGTTTCTTCAACTCAAATTCATCCGCC
TGATAATTTTCTTATATTTTCCTAAAGAAGGAAGAGAAGCGCATAGAGGA
GAAGGGAAATAATTTTTTAGGAGCCTTTCTTACGGCCTATGAGGAATTTG
GGGCTCAGTTGAAAAGCCTAAACTGCCTCTCGGGAGGTTGGGCGCGGCGA
ACTACTTTCAGCGGCGCACGGAGACGGCGTCTACGTGAGGGGTGATAAGT
GACGCAACACTCGTTGCATAAATTTGCgCTCCGCCAGCCCGGAGCATTTA
GGGGCGGTTGGCTTTGTTGGGTGAGCTTGTTTGTGTCCCTGTGGGTGGAC
GTGGTTGGTGATTGGCAGGATCCTGGTATCCGCTACAG

In embodiments where there is more than one first nucleic acid module, each module may be operatively linked to a different promoter, so long as the promoter is active in the producer cells and CD34+ cells.

The cassette or a vector derived therefrom, may comprise a second nucleic acid module encoding a CD46 binding adenoviral fiber polypeptide. No promoter is required on the cassette to drive expression of the second nucleic acid module; instead, expression is driven by the adenovirus major late promoter in the helper virus when HD-Ad is produced in the helper cells.

As used herein, the term “fiber polypeptide” means a polypeptide that comprises:
(a) an N-terminal tail domain or equivalent thereof, which interacts with the penton base protein of the capsid and contains the signals necessary for transport of the protein to the cell nucleus;
(b) one or more shaft domains or equivalents thereof; and
(c) a C-terminal knob domain or equivalent thereof that contains the determinants for receptor binding.

The fiber polypeptides spontaneously assemble into homotrimers, referred to as “fibers.” which are located on the outside of the adenovirus virion at the base of each of the twelve vertices of the capsid. As used herein, the term “fiber” refers to the homotrimeric protein structure composed of three individual fiber polypeptides. The adenovirus fiber mediates contact with, and internalization into, the target host cell.

As used herein, the term “fiber knob” refers to the C-terminal domain of the fiber polypeptide that is able to form into a homotrimer that binds to CD46. The C-terminal portion of the fiber protein can trimerize and form a fiber structure that binds to CD46. Only the fiber knob is required for CD46-targeting. Thus, the second nucleic acid module encodes an adenoviral fiber comprising one or more human adenoviral knob domain, or equivalent thereof, a bind to CD46. When multiple knob domains are encoded, the knob domains may be the same or different, so long as they each bind to CD46. As used herein, a knob domain “functional equivalent” is knob domain with one or more amino acid deletions, substitutions, or additions that retains binding to CD46 on the surface of CD34+ cells. Homotrimer formation can be determined according to methods well known to the practitioners in the art. For example, winterization of the fiber knob proteins can be assessed by criteria including sedimentation in sucrose gradients, resistance to trypsin proteolysis, and electrophoretic mobility in polyacrylamide gels (Hong and Engler, Journal of Virology 70:7071.-7078 (1996)). Regarding electrophoretic mobility, the fiber knob domain homotrimer is a very stable complex and will run at a molecular weight consistent with that of a trimer when the sample is not boiled prior to SDS-PAGE. Upon boiling, however, the trimeric structure is disrupted and the protein subsequently runs at a size consistent with the protein monomer. Trimerization of the fiber knob proteins can also be determined using the rabbit polyclonal anti-His6-HRP antibody as described in Wang H., et al., Journal of Virology 81:12785-12792 (2007).

In various embodiments, the knob domain is selected from the group consisting of an Ad11 knob domain, an Ad16 knob domain, an Ad21 knob domain, an Ad35 knob domain, an Ad50 knob domain, and functional equivalents thereof.

In various further embodiments, the knob domain comprises or consists of the amino acid sequence of one or more of the following, or functional equivalents thereof:

Ad11:
(SEQ ID NO: 94)
WTGVNPTEANCQIMNSSESNDCKLILTLVKTGALVTAFVYVIGVSNNFNM
LTTHRNINFTAELFFDSTGNLLTRLSSLKTPLNHKSGQNMATGAITNAKG
FMPSTTAYPFNDNSREKENYIYGTCYYTASDRTAFPIDISVMLNRRAIND
ETSYCIRITWSWNTGDAPEVQTSATTLVTSPFTFYYIREDD;
Ad16:
(SEQ ID NO: 95)
WTGAKPSANCVIKEGEDSPDCKLTLVLVKNGGLINGYITLMGASEYTNTL
FKNNQVTIDVNLAFDNTGQIITYLSSLKSNLNFKDNQNMATGTITSAKGF
MPSTTAYPFITYATETLNEDYIYGECYYKSTNGTLFPLKVTVTLNRRMLA
SGMAYAMNFSWSLNAEEAPETTEVTLITSPFFFSYIREDD;
Ad21
(SEQ ID NO: 96)
WTGIKPPPNCQIVENTDTNDGKLTLVLNVKNGGLVNGYVSLVGVSDTVNQ
MFTQKSATIQLRLYFDSSGNLLTDESNLKIPLKNKSSTATSEAATSSKAF
MPSTTAYPFNTTTRDSENYIHGICYYMTSYDRSLVPLNISIMLNSRTISS
NVAYAIQFEWNLNAKESPESNIATLTTSPFFFSYIREDDN;
Ad35
(SEQ ID NO: 97)
WTGINPPPNCQIVENTNTNDGKLTLVLVKNGGLVNGYVSLVGVSDTVNQM
FTQKTANIQLRLYFDSSGNLLTDESDLKIPLKNKSSTATSETVASSKAFM
PSTTAYPFNTTTRDSENYIHGICYYMTSYDRSLFPLNISIMLNSRMISSN
VAYAIQFEWNLNASESPESNIATLTTSPFFFSYITEDDN;
and
Ad50
(SEQ ID NO: 98)
WTGIKPPPNCQIVENTDTNDGKLTLVLVKNGGLVNGYVSLVGVSDTVNQM
FTQKSATIQLRLYFDSSGNLLTDESNLKIPLKNKSSTATSEAATSSKAFM
PSTTAYPFNTTTRDSENYIHGICYYMTSYDRSLVPLNISIMLNSRTISSN
VAYAIQFEWNLNAKESPESNIATLTTSPFFFSYIREDDN.

In another embodiment, the adenoviral knob domain comprises the amino acid sequence of SEQ ID NO: 100, which has been shown to possess improved CD46 binding capability (See U.S. Pat. No. 8,753,639).

Wt Ad35:
WTGINPPPNCQIVENTNTNDGKLTLVLVKNGGLVNGYVSLVGVSDTVNQM
FTQKTANIQLRLYFDSSGNLLTD/GESDLKIPLKNKSSTATSETVASSKA
FMPSTTAYPFNTTAT/ATRDSENYIHGI/LCYYMTSYDRSLFPLNISIML
NSRMISSNVAYAIQFEWNLNASESPESNIATLTTSPFFFSYITEDDN
(SEQ ID NO: 99, 101 (wild type Ad35 knob). SEQ ID
NO: 100 (mutant Ad35 knobb)).

In another embodiment, the second nucleic acid module encodes an adenoviral fiber polypeptide comprising one or more human adenoviral shaft domain or functional equivalents thereof. Since the shaft domain is not critical for CD46 binding, the shaft domain can be derived from any adenoviral serotype. Thus, the one or more shaft domains may comprise or consist of one or more shaft domains from human adenoviral serotypes 1, 2, 3, 4, 5, 6, 7, 8, 9, 10, 11, 12, 13, 14, 15, 16, 17, 18, 19, 20, 21, 22,23, 24, 25, 26, 27, 28, 29, 30, 31, 32, 33, 34, 35, 36 37, 38, 39, 40, 41, 42, 43, 44, 45, 46, 47, 48, 49, 50, 51, 52, 53, 54, 55, 56, 57, combinations thereof or functional equivalents thereof. As used herein, a “functional equivalent” of a shaft domain is any portion of a shaft domain or mutant thereof, that permits fiber knob trimerization.

In one embodiment, each shaft domain or shaft domain motifs selected from the group consisting of Ad5 shaft domains, Ad11 shaft domains, Ad16 shaft domains, Ad21 shaft domains, Ad35 shaft domains, Ad50 shaft domains, and functional equivalents thereof, combinations thereof, and functional equivalents thereof. The shaft domain is required for fiber knob trimerization, which is required for binding to CD46. Such equivalents can be readily determined by those of skill in the art. For example, surface plasmon resonance (SPR) studies using sensors containing immobilized recombinant CD46 can be used to determine if recombinant polypeptides being assessed bind to CD46, combined with CD46 competition studies.

The shaft domain may comprise any suitable number, for example between 1 and 22, shaft domains or equivalents thereof. Thus, in various embodiments to shaft domain comprises 1-22, 1-21, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, 1-2, 2-22, 2-21, 2-20, 2-19, 2-18, 2-17, 2-16, 2-15, 2-14, 2-13,2-12, 2-11, 2-10, 2-9, 2-8, 2-7, 2-6, 2-5, 2-4, 2-3, 3-22, 3-21, 3-20, 3-19, 3-18, 3-17, 3-16, 3-15, 3-14, 3-13, 3-12, 3-11, 3-10, 3-9, 3-8, 3-7, 3-6, 3-5, 3-4, 4-22, 4-21, 4-20, 4-19, 4-18, 4-17, 4-16, 4-15, 4-14, 4-13, 4-12, 4-11, 4-10, 4-9, 4-8, 4-7, 4-6, 4-5, 5-22, 5-21, 5-20, 5-19, 5-18, 5-17, 5-16, 5-15, 5-14, 5-13, 5-12, 5-11, 5-10, 5-9, 5-8, 5-7, 5-6, 6-22, 6-21, 6-20, 6-19, 6-18, 6-17, 6-16, 6-15, 6-14, 6-13, 6-12, 6-11, 6-10, 6-9, 6-8, 6-7, 7-22, 7-21, 7-20, 7-19, 7-18, 7-17, 7-16, 7-15, 7-14, 7-13, 7-12, 7-11, 7-10, 7-9, 7-8, 8-22, 8-21, 8-20, 8-19, 8-18, 8-17, 8-16, 8-15, 8-14, 8-13, 8-12, 8-11, 8-10, 8-9, 9-22, 9-21, 9-20, 9-19, 9-18, 9-17, 9-16, 9-15, 9-14, 9-13, 9-12, 9-11, 9-10, 10-22, 10-21, 10-20, 10-19, 10-18, 10-17, 10-16, 10-15, 10-14, 10-13, 10-12, 10-11, 11-22, 11-21, 11-20, 1-19, 11-18, 11-17, 11-16, 11-15, 11-14, 11-13, 11-12, 12-22, 12-21, 12-20, 12-19, 12-18, 2-17, 12-16, 12-15, 12-14, 12-13, 13-22, 13-21, 13-20, 13-19, 13-18, 13-17, 13-16, 13-15, 13-14, 14-22, 14-21, 14-20, 14-19, 14-18, 14-17, 14-16, 14-15, 15-22, 15-21, 15-20, 15-19, 15-18, 15-17, 15-16, 16-22, 16-21, 16-20, 16-19, 16-18, 16-17, 17-22, 17-21, 17-20, 17-19, 17-18, 18-22, 18-21, 18-20, 18-19, 19-22, 19-21, 19-20, 20-22, 20-21, 21-22, 1, 2, 3, 4, 5, 6, 7, 8, 9, 10, 11, 12, 13, 14, 15, 16, 17, 18, 19, 20, 21, or 22 shaft domains or equivalents thereof. Where more than 1 shaft domain or equivalent is present, each shaft domain or equivalent can be identical, or one or more copies of the shaft domain or equivalent may differ in a single recombinant polypeptide. In one embodiment, the cassette encodes a single shaft domain or equivalent.

In another embodiment, the one or more shaft domains comprise an amino acid sequence selected from the group consisting of the following, combinations thereof, or equivalents thereof.

Ad11P fiber,
(SEQ ID NO: 118)
NGVLTLKCLTPLTTTGSLQLKVGGGLTVDDTNGFLKENISATTPLVKTGH
SIGLPLGAGLGTNENKLCIKLGQGLTFNSNNICIDDNINTL;
AD16,
(SEQ ID NO: 119)
DGVLTLKCNVPLTTASGPLQLKVGSSLTVDTIDGSLEENITAAAPLTKTNH
SIGLLIGSGLQTKDDKLCLSLGDGLVTKDDKLCLSLGDGLITKNDVLCAKL
GHGLVFDSSNAITIENNTL;
AD21,
(SEQ ID NO: 120)
DGVLTLNCLTPLTTTGGPLQLKVGGGLIVDDTDGTLQENIRATAPITKNNH
SVELSIGNGLETQNNKLCAKLGNGLKFNNGDICIKDSINTL;
AD35,
(SEQ ID NO: 121)
DGVLTLKCLTPLTTTGGSLQLKVGGGLTVDDTDGTLQENIRATAPITKNNH
SVELSIGNGLETQNNKLCAKLGNGLKFNNGDICIKDSINTL;
AD50,
(SEQ ID NO: 122)
DGVLTLNCLTPLTTTGGPLQLKVGGGLIVDDTDGTLQENIRVTAPITKNNH
SVELSIGNGLETQNNKLCAKLGNGLKFNNGDICIKDSINTL;
AD5
(SEQ ID NO: 105)
PGVLSLRLSEPLVTSNGMLALKMGNGLSLDEAGNLTSQNVTTVSPPLKKTK
SNINLEISAPLTVTSEALTVAAAAPLMVAGNTLTMQSQAPLTVHDSKLSIA
TQGPLTVSEGKLALQTSGPLTTTDSSTLTITASPPLTTATGSLGIDLKEPI
YTQNGKLGLKYGAPLHVTDDLNTLTVATGPGVTINNTSLQTKVTGALGFDS
QGNMQLNVAGGLRIDSQNRRLILDVSYPFDAQNQLNLRLGQGPLFINSAHN
LDINYNKGLYLFTASNNSKKLEVNSLTAKGLMFDATAINAINAGDGLEFGS
PNAPNTNPLKTKIGHGLEFDSNKAMVPKLGTGLSFDSTGAITVGNKNNDKL
TL.

In another embodiment, one or more (or all) shaft domains or equivalents comprise or consist of an amino acid sequence according to SEQ ID NO 123:

GVL(T/S)LKC(L/V)(T/N)PLTT(T/A)(G/S)GSLQLKVG(G/S)
GLTVD(D/T)T(D/N)G(T/F/S)L(Q/K/E)ENI(G/S/K)(A/V)
(T/N)TPL(V/T)K(T/S)(G/N)HSI(G/N)L(S/P)(L/I)G
(A/P/N)GL(G/Q)(T/I)(D/E)(E/Q)NKLC(T/S/A)KLG(E/Q/N)
GLTF(N/D)S(N/S)N(I/S)(C/I(I/A)(D/N/L)(D/K)N(I/-)
NTL;

### or SEQ NOS:124-129:

Ad3 shaft domain motif: NSIALKNNTL SEQ ID NO: 124
Ad7 shaft domain motif: NSNNICINDNINTL SEQ ID NO: 125

Ad5 shaft domain motif: GAITVGNKNNDKLTL SEQ ID NO: 126
Ad11 shaft domain motif: NSNNICIDDNINTL SEQ ID NO: 127
Ad14 shaft domain motif: NSNNICIDDNINTL SEQ ID NO: 128
Ad35 shaft domain motif: GDICIKDSINTL SEQ ID NO: 129.

In this sequence and other variable sequences shown herein, the variable residues are noted within parentheses, and a “−” indicates that the residue may be absent.

In another embodiment, one or more (or all) shaft domains or equivalents comprise or consist of an amino acid sequence according to SEQ ID NO 130:

GVLITKCLTPLTTTGGSLQLKVGGGLT(V/I)DDTDG(T/F)L(Q/K)
ENI(G/S)ATTPLVKTGHSIGL(S/P)LG(A/P)GLGT(D/N)ENKLC
(T/A)KLG(E/Q)GLTFNSNNICI(D/N)DNINTL.;
or
SEQ ID NOS: SEQ ID NOS:124-129

In a still further embodiment, one or more or all shaft domains or shaft domain motifs in the recombinant polypeptide comprise or consist of an amino acid sequence selected from the group consisting of SEQ ID NO:152 (Ad3), SEQ ID NO: 153 (Ad7), SEQ ID NO: 154 (Ad11), SEQ ID NO: 155 (Ad14) SEQ ID NO:156 (Ad14a), and SEQ ID NOS:124-129.

In a further embodiment, the second nucleic acid module encodes an adenoviral fiber polypeptide comprising a human adenoviral tail domain, or equivalent thereof. As used herein, a functional equivalent of an adenoviral tail domain is a mutant that retains the ability to interact with the penton base protein of the capsid (on a helper Ad virus) and contains the signals necessary for transport of the protein to the cell nucleus. The tail domain used is one that will interact with the penton based protein of the helper Ad virus capsid being used for HD-Ad production. Thus, if an Ad5 helper virus is used, the tail domain will be derived from Ad5; if an Ad35 helper virus is used, the tail domain will be from Ad 35, etc.

In one embodiment, the tail domain is selected front the group consisting of an Ad11 tail domain, an Ad16 tail domain, an Ad21 tail domain, an Ad35 tail domain, an Ad50 tail domain, and functional equivalents thereof. In another embodiment, the tail domain comprises the amino acid sequence of one of the following proteins:

Ad11P
(SEQ ID NO: 131)
MTKRVRLSDSFNPVNTYEDESTSQHPFINPGFISPNGFTQSP;
AD16
(SEQ ID NO: 132)
MAKRARLSSSFNPVYPYEDESSSQHPFINPGFISSNGFAQSP;
AD21
(SEQ ID NO: 131)
MTKRATRESDSENPVYPYEDESTSQHPFINPGFISPNGENSP;
AD35
(SEQ ID NO: 131)
MTKRVRLSDSFNPVYPYEDESTSQHPFINPGFISPNGFTQSP;
AD50
(SEQ ID NO: 131)
MTKRVRLSDSFNPVYPYEDESTSQHPFINPGFISPNGFTQSP.

The cassette, or a vector derived therefrom, may comprise an inverted terminal repeat (ITR) at each terminus of the recombinant nucleic acid vector, wherein the ITR derived from a CD46-binding adenovirus serotype, that aid in concatamer formation in the nucleus after the single-stranded HD-Ad viral DNA is converted by host cell DNA polymerase complexes into double-stranded DNA. The ITRs are typically between about 100-150 nucleotides in length. Thus, in one embodiment the ITRs are from Ad11, Ad16, Ad21, Ad35, or Ad50. in another embodiment, the ITRs comprise or consist of the sequence of one of the following:

Ad11p, ACCESSION NC_011202
5′ ITR:
(SEQ ID NO: 133)
CATCATCAATAATATACCTTATAGATGGAATGGTGCCAATATGTAAATGA
GGTGATTTTAAAAGTGTGGATCGTGTGGTGATTGGCTGTGGGGTTAACGG
CTAAAAGGGGCGGTGCGACCGTGGGAAAATGACGTT
AD16, ACCESSION NUMBER AY601636
5′ ITR
(SEQ ID NO: 134)
CATTATCTATAATATACCTTATAGATGGAATGGTGCCAACATGTAAATGA
GGTAATTTAAAAAAGTGCGCGCTGTGTGGTGATTGGCTGCGGGGTGAACG
GCTAAAAGGGGCGG
AD21, ACCESSION KF528688
5′ ITR
(SEQ ID NO: 135)
TATTATATAATATACCTTATAGATGGAATGGTGCCAATATGCAAATGAGG
TAATTTAAAAAAGTGCGCGCTGTGTGGTGATTGGCTGCGGGGTGAACGGC
TAAAAGGGGCGG
AD35, ACCESSION NC 000019
5′ ITR
(SEQ ID NO: 136)
CATCATCAATAATATACCTTATAGATGGAATGGTGCCAATATGTAAATGA
GGTGATTTTAAAAAAGTGTGGGCCGTGTGGTGATTGGCTGTGGGGTTAAC
GGTTAAAAGGGGCGGCGCGGCCGTGGGAAAATGACGTT
AND
AD50, ACCESSION AY737798
5′ ITR
(SEQ ID NO: 137)
CAATCAATATAATATACCTTATAGATGGAATGGTGCCAATATGTAAATGA
GGTAATTTAAAAAAGTGCGCGCTGTGTGGTGATTGGCTGCGGGGTGAACG
GCTAAAAGGGGCGG.

The cassette, or a vector derived therefrom, may comprise a packaging signal from a CD46-binding adenovirus serotype. Thus, in one embodiment, the packaging signals are from Ad11, Ad16, Ad21, Ad35, or Ad50. In another embodiment, the packaging signals comprise or consist of the sequence of one of the following (SEQ ID NO: 139-141), wherein SEQ ID NO:139 is the Ad5 packaging signal, SEQ ID NO: 140 is an Ad35 packaging signal, and SEQ ID NO:141 is a consensus sequence of AD5/35 packaging signal.

In another embodiment, the packaging signal is flanked by nucleic acid excision signals, including but not limited to loxP sites (for use with Cre recombinase) of ftr sites (for use with Flp recombinase). This embodiment facilitates removal of helper virus from HD vector preparations based, for example, on Cre- or Flp-recombinase-mediated excision of the packaging signal flanked by loxP sites during coinfection.

The cassettes of the invention, and production vectors derived therefrom, are particularly useful for the production of helper-dependent adenovirus (HD Ad), which can be used for gene therapy. In one embodiment, the cassette encodes no other adenoviral proteins, which is optimal for gene therapy applications, to avoid the Hd Ad propagation after administration to a gene therapy patient, as well as any other potential toxicity issues.

In another embodiment, the cassette, or a vector derived therefrom, may further comprise a transgene operatively linked to a promoter that is active in CD34+ cells. Any suitable promoter may be used, such as those described herein. This embodiment permits use of the cassettes, or vectors derived therefrom, as gene therapy vehicles. The insert capacity of HD-Ad vectors is 30 kb which allows the accommodation of several ENs and homologous donor templates. This is important for the simultaneous editing of multiple genes in HSCs for gene therapy purposes or to establish relevant models for multigenic human diseases. In this embodiment, the nuclease creates a DNA break in a CD34+ cell genomic target of interest, to permit transgene genomic integration.

In one embodiment first recombination site and a second recombination site flank the transgene, wherein the first recombination site and a second recombination site target a site in CD34+ cell genomic DNA flanking a desired insertion site for the transgene. Thus, standard homologous recombination techniques can be used for genomic integration of the transgene(s) of interest. It is well within the level of those of skill in the art to determine appropriate recombination sites to use in the cassette, based on the genomic target site of interest.

The cassette or vectors derived therefrom are preferably at least 28 kb in length, and may be 28-35 kb in length. Any suitable nucleic acid sequences can be used as “stuffer” sequences, as is known to those of skill in the art. In one non-limiting embodiment, the stuffer DNA may comprise scrambled human X-chromosomal DNA.

The nucleic acid cassette may be any DNA or RNA, and can be prepared and isolated using standard molecular biological techniques, based on the teachings herein. The nucleic acids may comprise additional domains useful for promoting expression and/or purification of the cassette.

In a further aspect, the invention provides recombinant nucleic acid vectors comprising the nucleic acid cassettes of the invention. Any suitable vector can be used, including but not limited to plasmid vectors. In some embodiments the vector is a shuttle vector (such as a shuttle plasmid), which includes a part of the desired HD-Ad genome (i.e.: at least the first nucleic acid module, and optionally also the second nucleic acid module and transgene(s)). Such shuttle vectors can be used to produce large quantities of the nucleic acid vector, which can then be used to subclone desired regions of the expression cassette into a production vector. In one embodiment, the shuttle vector includes the first nucleic acid module, which can subsequently be cloned into a production vector that includes the second nucleic acid module. ITRs, stuffer sequences, packaging signals, and/or transgene(s). In another embodiment, the shuttle vector includes the first and second nucleic acid modules, which can then be cloned into a production vector that includes ITRs, staffer sequences, packaging signals, and/or transgene(s). In a still further embodiment, the shuttle vector includes the first and second nucleic acid modules and the transgene(s), which can then be cloned into a production vector that includes ITRs, stuffer sequences, and packaging signals. Selection of suitable shuttle vectors and production vectors (such as plasmid vectors) is well within the level of those of skill in the art, based on the teachings herein.

In another aspect, the invention provides recombinant host cells, comprising the expression cassette of any embodiment or combination of embodiments of the invention. The recombinant host cells may be any suitable host cell in which the cassettes can be expressed, and are preferably producer cells as described herein, including but not limited to human embryonic kidney (HEK) 293 cells, HEK 293-Cre cells, PerC6 cells, HCT 116 cells, etc. In one embodiment, the producer cells are HEK 293 cells or HEK 293-Cre cells. The recombinant host cell may produce the miRNA to which the miRNA target sites encoded by the cassette bind.

In a further embodiment, the host cell further comprises helper adenovirus. Growth of HD-Ad vectors of the invention depends on co-infection of the producer cells with helper Ad vector, which provides all necessary Ad proteins in trans (i.e.: all viral proteins except proteins encoded by the E1 and E3 regions), and also provides the adenoviral promoter sequences (i.e., the Ad major late promoter) necessary for expression of the Ad fiber polypeptide genes on the cassette. The use of helper adenoviruses for production of helper-dependent adenoviruses is well understood in the art (see, for example, Kochanek, S., G. Schiedner, and C. Volpers, 2001. Curr Opin Mol Thor 3:454-463). In one embodiment, after cloning a transgene-containing expression cassette into an HD-Ad production plasmid, the construct is linearized and transfected into the cells of the HD-Ad producer cells, which are subsequently infected with the helper virus. After a suitable number (such as 3) of serial pre-amplification steps, large-scale HD-Ad production is performed in suspension culture. For purification, virus is isolated by cesium chloride gradients using ultracentrifugation.

Thus, in another aspect, the invention provides methods for making the HD-Ad virus of the invention, comprising culturing a recombinant host cell of the invention that has been transduced with helper adenovirus, under conditions suitable to promote expression of genes on the expression cassette and the helper adenovirus sufficient to assemble the helper dependent adenovirus. It is well within the level of those of skill in the art, based on the disclosure herein, to determine appropriate conditions for culturing the recombinant host cells of the invention to promote expression of genes on the expression cassette and the helper adenovirus sufficient to assemble the helper dependent adenovirus. Removal of helper virus from HD vector preparations can be carried out using any suitable technique. Non-limiting exemplary conditions are provided in the examples that follow. In one embodiment, where the cassette comprises loxP excision signals flanking the packaging site isolation may comprise use of Cre-recombinase-mediated excision of the packaging signal flanked by loxP sites during coinfection. In this embodiment HD-Ad amplification may be done in cells expressing Cre recombinase (such as 293-Cre).

In another aspect, the invention provides recombinant helper dependent adenovirus comprising the expression cassette of any embodiment or combination of embodiments of the invention as a genome. The recombinant helper dependent adenovirus can be made using any suitable method, including those disclosed herein.

In another aspect, the invention provides methods for hematopoietic cell gene therapy, comprising in vivo transduction of hematopoietic cells mobilized from bone marrow into peripheral blood of a subject in need of hematopoietic cell gene therapy with a recombinant helper dependent Ad virus of any embodiment or combination of embodiments of the invention, wherein the nuclease targets a hematopoietic cell genomic gene to be disrupted, wherein disruption of the hematopoietic cell genomic gene provides a therapeutic benefit to the subject.

The inventors have developed a new in vivo approach for HSC gene editing/therapy, based on the mobilization of CD34+ hematopoietic cells (such as hematopoietic stem cells (HSCs) from the bone marrow into the peripheral blood stream and the administration (such as by intravenous injection) of a helper-dependent adenovirus vector of any embodiment or combination of embodiments of the present invention. The cellular receptor for the Hd-Ad vectors of the invention is CD46, a protein that is uniformly expressed at high levels on human HSCs. The methods result in Hd-Ad transduction of the mobilized CD34+ cells, rehoming of the transduced CD34+ cells to the bone marrow, and long term persistence of the transduced cells, such as HSCs as a source of all blood cell lineages.

The HD-Ad vector platform of the present invention for EN gene delivery to HSCs has major advantages over other delivery systems. i) It allows for efficient targeting of primitive HSCs with less cytotoxicity. ii) The insert capacity of HD-Ad vectors is 30 kb which allows the accommodation of several ENs and homologous donor templates. This is useful for the simultaneous editing of multiple genes in HSCs for gene therapy purposes or to establish relevant models for multigenic human diseases. The use of HD-AD vectors also makes it possible to combine both the EN expression cassette and the donor transgenes with extended homology regions into one vector. In this context is notable that the efficacy of homologous recombination directly correlates with the length of the homology regions. HD-Ad vectors of the invention allow for the transduction of target cells in vivo. Our preliminary studies in human CD34+/NOG and human CD46-transgenic mice show that the HD-Ad vectors of the invention can transduce mobilized HSCs after intravenous injection.

Transduction rates are influenced by several factors, including target cell accessibility. Without HSC mobilization, administration of the HD-Ad of the invention (such as by intravenous injection) will not result in transduction of CD34+ cells.

In the examples that follow, we have shown in human CD46 transgenic (hCD46tg) mice and NOG mice with engrafted human HSCs (NOG/hCD34+) that in vivo transduced HSCs home back to the bone marrow where they remain functional HSCs. At day 3 after in vivo transduction, up to 15% of bone marrow-localized HSCs expressed the transgene.

Any suitable method for mobilization of CD34+ hematopoietic cells (such as HSCs) into the peripheral blood can be used. In various non-limiting embodiments, the subject is administered mobilization agents selected from the group consisting of Granulocyte colony stimulating factor (GCSF), Plerixafor (AMD3100; a CXCR inhibitor), POL5551 (a CXCR4 antagonist) (Karpova et al., Leukemia (2013) 27, 2322-2331) BIO5192 (small molecule inhibitor of VLA-4) (Ramirez, et al., 2009. Blood 114:1340-1343), and combinations thereof. In specific embodiments, the mobilization agents may be combined as follows:
(a) Granulocyte colony stimulating factor (GCSF)+ Plerixafor (AMD3100; a CXCR inhibitor);
(b) GCSF+ POL5551 (a CXCR4 antagonist); and
(c) GSCF+ BIO5192 (small molecule inhibitor of VLA-4).

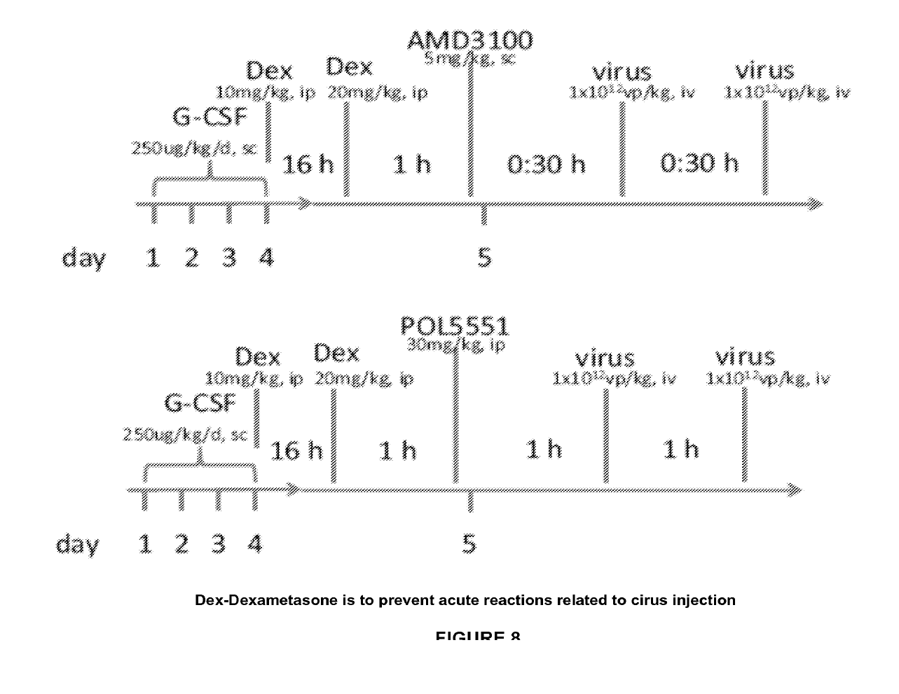

Mobilization may be achieved using the mobilization agents as deemed most appropriate under all circumstances as determined by attending medical personnel. As will be understood by those of skill in the art, the mobilization agents may be administered once or more (i.e.:1, 2, 3, 4, 5, 6, or more times); such administration be multiple times in a single day or spread out over multiple days. Dosage ranges for the mobilization agents may be determined by those attending medical personnel based on all circumstances. Similarly, HD-Ad may be may be administered once or more (i.e.:1, 2, 3, 4, 5, 6, or more times); such administration be multiple times in a single day or spread out over multiple days. Dosage ranges for the HD-Ad may be determined by those attending medical personnel based on all circumstances. As will be further understood by those of skill in the art, treatment may comprise 1 or multiple rounds of mobilization/HD-Ad administration. In various non-limiting embodiments, HD-Ad can be administered approximately 1 hour after AMD3100-based mobilization or approximately 2 hours after POL5551-based mobilization. A further non-limiting and exemplary treatment schedule is shown in FIG. 8.

The subject may be any mammalian subject in need of hematopoietic cell gene therapy, including but not limited to primates, rodents, dogs, cats, horses, etc. In one embodiment, the subject is a mammal, such as a human. The subject may be suffering from a hematopoietic cell disorder (therapeutic gene therapy), or may be at risk of such a disorder (prophylactic gene therapy). Exemplary such hematopoietic cell disorders include, but are not limited to, β-thalassemias, human immunodeficiency virus infection and/or acquired immunodeficiency syndrome, Ebola virus infection, Epstein-Barr virus infection, and sudden acute respiratory syndrome virus (SARS) infection. In each case, the subject may already have the disorder, or may be at risk of the disorder.

For example, there are two co-receptors of CD4 for HIV infection, CCR5 and CXCR4. HIV isolated from infected individuals early after infection are predominantly CCR5-tropic, indicating a selective advantage of these viruses during the early stages of infection (54, 61). A homozygous Δ32 deletion in the ccr5 gene, found in about 1% of Caucasians, confers a natural resistance to HIV-1 (4, 63). Individuals carrying this mutation are healthy, most likely due to the redundant nature of the chemokine system. In a recent study it was shown that transplantation of hematopoietic stem/progenitor cells (HSCs) from a donor who was homozygous for ccr5 Δ32 in a patient with acute ⁻myeloid leukemia and HIV-1 infection resulted in long-term control of HIV (49). Thus, methods of the present invention can be used to eliminate CCR5 in HSCs (CD34+ cells). Since HSCs are a source for all blood cell lineages, ccr5 knock-out would not only protect CD4+ cells descendant from the transduced HSCs, but also all remaining lymphoid and myeloid cell types that are potential targets for HIV infection. In contrast to CD4+ cell transplants, which have a relatively limited in vivo life span, a single HSC transplant would allow long-term protection or control of HIV/AIDS. In this embodiment, the HD-Ad nuclease is capable of generating a DNA break in the gene encoding CCR5; in one non-limiting embodiment, the nuclease comprises or consists of the nuclease of SEQ ID NO: 91-93, and the methods could be used to treat or limit development of AIDS in a subject that has been infected with HIV, or is at risk of developing HIV (including but not limited to commercial sex workers, injection drug users, people in serodiscordant relationships and members of high-risk groups who choose not to use condoms).

As will be understood by those of skill in the art, similar techniques could be used to treat or limit development of Ebola (nuclease targeting Niemann-Pick disease, type C1 receptor (NPC1)) and SARS (nuclease targeting angiotensin-converting enzyme 2 receptor (ACE2)), as well as any other disorder that can be treated or limited by inhibiting/eliminating expression of a gene in HSCs.

As used herein, the term “treat,” “treatment,” or “treating,” means to reverse, alleviate, ameliorate, inhibit, slow down or stop the progression Of severity of a symptom or condition of the disorder being treated. The term “treating” includes reducing or alleviating at least one adverse effect or symptom of a condition. Treatment is generally “effective” if one or more symptoms are reduced. Alternatively, treatment is “effective” if the progression of a condition is reduced or halted. That is, “treatment” may include not just the improvement of symptoms, but also a cessation or slowing of progress or worsening of symptoms that would be expected in the absence of treatment. Beneficial or desired clinical results include, but are not limited to, alleviation of one Of more symptom(s), diminishment of extent of the deficit, stabilized (i.e., not worsening) state of the disorder, delay or slowing of the disorder, and an increased lifespan as compared to that expected in the absence of treatment.

As used herein, the term “administering,” refers to the placement of the recombinant helper dependent Ad virus into a subject by a method or route deemed appropriate. The HD-Ad may be administered as part of a suitable pharmaceutical formulation; any pharmaceutically acceptable formulation can be used, including but not limited to saline or phosphate buffered saline. The therapeutic can be administered by any appropriate route which results in an effective treatment in the subject including intravenous administrations. Dosage regimens can be adjusted to provide the optimum desired response (e.g., a therapeutic or prophylactic response). A suitable dosage range may, for instance, be 2×10e10 vp/kg. The recombinant helper dependent Ad virus can be delivered in a single bolus, or may be administered more than once (e.g., 2, 3, 4, 5, or more times) as determined by an attending physician.

In another aspect, the invention provides methods for hematopoietic cell gene therapy, comprising in vivo transduction of hematopoietic cells mobilized into peripheral blood of a subject in need of hematopoietic cell gene therapy with the recombinant helper dependent Ad virus of any embodiment of combination of embodiments of the invention, wherein the recombinant nucleic acid expression cassette comprises a transgene operatively linked to a promoter that is active in CD34+ cells, wherein the transgene is flanked by at least a first recombination site and a second recombination site, wherein the first recombination site and a second recombination site target a site in the hematopoietic cell genomic DNA flanking a desired insertion site for the transgene, and wherein insertion of the transgene into the desired insertion site provides a therapeutic benefit to the subject.

This aspect is similar to the methods described above, but comprises targeted transgene insertion into the CD34+ genome (instead of, or in combination with the targeted gene disruption disclosed above), to treat or limit development of a disorder susceptible to treatment by hematopoietic gene therapy.

For example, the β-thalassemias are congenital hemolytic anemias that are caused by mutations that reduce or abolish the production of the β-globin chain of adult hemoglobin. This deficiency causes ineffective erythropoiesis and hemolytic anemia. For patients lacking a matched donor, globin gene therapy offers a cure. Thus, the methods of the invention may comprise use of an HD-Ad vector in which the transgene is a β-globin gene, gamma-globin gene, globin LCR, antibody gene, T-cell receptor gene, chimeric antigen-receptor gene.

In one embodiment of any of the methods of the invention, the recombinant helper dependent Ad virus is administered by intravenous injection. In another embodiment, one or more copies of the miRNA are selected from the group consisting of SEQ ID NOS: 1-90. In a further embodiment, the nuclease is selected from the group consisting of zinc-finger nucleases (ZFNs), transcription activator-like effector nucleases (TALENs), meganucleases, and CRISPR-Cas9 nucleases. In another embodiment, the nuclease is capable of generating a DNA break in a CD34+ cell genomic target selected from the group consisting of genes encoding CCR5, β-globin, CR2 (EBV receptor), NPC1 (Ebola receptor), ACE2 (SARS receptor), and genes that encode proteins that can lead to lysosomal storage disease if misfolded. In a further embodiment, the nuclease comprises the amino acid sequence of 91-93.

In another aspect, the invention provides a recombinant nucleic acid comprising two or more copies of a miRNA target site that comprises or consists of the reverse complement of a nucleic acid sequence selected from the group consisting of SEQ ID NOS: 1-90. The miRNA target sites may all be the same, or may be different. In various embodiments, the recombinant nucleic acid comprises 2, 3, 4, 5, 6, 7, 8, 9, 10, or more copies of the miRNA target. In one embodiment, the miRNA target sites in total comprise target sites for at least two different miRNAs.

The recombinant nucleic acids of this aspect of the invention can be used, for example, as a module to fuse to any coding region of interest, such that upon expression in a cell expressing the miRNA that binds to the miRNA target site, the resulting fusion RNA will be degraded. Such cells include but are not limited to, viral producer cells such as HEK293 and HEK 293-Cre cells. The recombinant nucleic acids of this aspect can be used in the cassettes and HD-Ad vectors of the present invention, and may also be used, for example, in the production of any other viral vector produced in HEK293 and HEK 293-Cre cells, such as lentivirus and r AAV vectors.

In one embodiment, the recombinant nucleic acid includes at least one miRNA target site that binds to a miRNA comprising CACUGGUAGA (SEQ ID NO: 1) (has-miR183-5p core) (including but not limited to SEQ ID NOS: 10 to 39), and at least one miRNA target that binds to a miRNA comprising UGUGCUUGAUCUAA (SEQ ID NO: 2) (has-miR218-5p core) (including but not limited to SEQ ID NOS: 40 to 71). In another embodiment, the recombinant nucleic acid includes at least one miRNA target site that binds to a miRNA comprising CACUGGUAGA (SEQ ID NO: 1) (has-miR183-5p core) (including but not limited to SEQ ID NOS: 10 to 39), and at least one miRNA target that binds to a miRNA. comprising CACUAGCACA (SEQ ID NO: 3) (miR96-5p core) (including but not limited to SEQ ID NOS: 72 to 90). In a further embodiment, the recombinant nucleic acid includes at least one miRNA target site that binds to a miRNA comprising UGUGCUUGAUCUAA (SEQ ID NO: 2) (has-miR218-5p core) (including but not limited to SEQ ID NOS: 40 to 71) and at least one miRNA target that binds to a miRNA comprising CACUAGCACA (SEQ ID NO: 3) (miR96-5p core) (including but not limited to SEQ ID NOS: 72 to 90). In a further embodiment, the recombinant nucleic acid includes at least one miRNA target site that binds to a miRNA comprising CACUGGUAGA (SEQ ID NO: 1) (has-miR183-5p core) (including but not limited to SEQ ID NOS: 10 to 39), at least one miRNA target that binds to a miRNA comprising UGUGCUUGAUCUAA (SEQ ID NO: 2) (has-miR218-5p core) (including but not limited to SEQ ID NOS: 40 to 71), and at least one miRNA target that binds to a miRNA comprising CACUAGCACA (SEQ ID NO: 3) (miR96-5p core) (including but not limited to SEQ ID NOS: 72 to 90).

In one embodiment of any of the above embodiments, each copy of the miRNA target site is separated by a spacer that is not present together with the miRNA target site in a naturally occurring nucleic acid molecule. In various non-limiting examples, the spacer may be between 1-10, 2-9, 3-8, 4-7, or 5-6 nucleotides in length.

In a further embodiment, the invention provides a nucleic acid expression vector comprising the recombinant nucleic acids of this aspect of the invention operatively linked to a promoter sequence.

### EXAMPLE 1

### Abstract

Genome editing with site-specific endonucleases has implications for basic biomedical research as well as for gene therapy. We generated helper-dependent, capsid-modified adenovirus (HD-Ad5/35; Ad5 shaft (22 shaft domains), Ad5 tail and Ad 35 mutant knob domain (SEQ ID NO: 100)) vectors for zinc-finger nuclease (ZFN)- or Transcription Activator-Like Effector Nucleases (TALEN)-mediated genome editing in human CD34+ hematopoietic stem cells (HSCs) from mobilized adult donors. The production of these vectors required that ZFN and TALEN expression in HD-Ad5/35 producer 293-Cre cells was suppressed. To do this, we developed a miRNA-based system for regulation of gene expression based on miRNA expression profiling of 293-Cre and CD34+ cells. Using miR-183-5p and miR-218-5p based regulation of transgene gene expression, we first produced an HD-Ad5/35 vector expressing a ZFN specific to the HIV co-receptor gene ccr5. We demonstrated that HD-Ad5/35.ZFNmiR vector conferred ccr5 knock-out in primitive HSC (i.e. long-term culture initiating cells and NOD/SCID repopulating cells). The ccr5 gene disruption frequency achieved in engrafted HSCs found in the bone marrow of transplanted mice is clinically relevant for HIV therapy considering that these cells can give rise to multiple lineages, including all the lineages that represent targets and reservoirs for HIV. We produced a second HD-Ad5/35 vector expressing a TALEN targeting the DNase hypersensitivity region 2 (HS2) within the globin LCR. This vector has potential for targeted gene correction in hemoglobinopathies. The miRNA regulated HD-Ad5/35 vector platform for expression of site-specific endonucleases has numerous advantages over currently used vectors as a tool for genome engineering of HSCs for therapeutic purposes.

### Introduction

Hematopoietic stem cells (HSCs) are an important target for gene therapy. A major task in HSC gene therapy is the site-specific modification of the HSC genome using artificial site-specific endonucleases (EN) that target a DNA break to preselected genomic sites. ENs are employed to knock-out genes, correct frame shift mutations, or to knock-in a wild-type cDNA into the endogenous site or heterologous sites. There are now a number of different EN platforms to generate site-specific DNA breaks in the genome [1]. One group of ENs contains DNA binding protein domains. This group includes meganucleases with DNA binding and nuclease properties as well as ZFNs and TALENs in which the DNA binding domain is fused with the bacterial endonuclease FokI. Because DNA cleavage by FokI requires two FokI molecules bound to each of the DNA strands, two subunits of the FokI containing ENs have to be expressed. A second group of ENs is based on RNA-guided DNA recognition and utilizes the CRISPR/Cas9 bacterial system. Several approaches have been used to deliver EN expression cassettes to HSCs. Because it is thought that the ENs need to be expressed only for a short time to achieve permanent modification of the target genomic sequence, most of the EN cassette delivery systems allow only for transient expression of ENs without integration of the EN gene into the host genome.

Among our attempts to produce CCR5 ZFN-expressing HD-Ad vectors was a vector that allowed for Tet-inducible transgene expression using a fusion of the Krüppel-associated box (KRAB) domain and the tetracycline repressor. We produced GFP expressing HD-Ad5/35 vectors and showed that background expression in 293 cells with Tet induction was suppressed. However, when we replaced that GFP gene with the CCR5 ZFN gene, the resulting HD-Ad genomes isolated from purified particles demonstrated genomic rearrangements and a deletion of parts of the ZFN cassette (Data not shown).

To generate HD-Ad5/35 vectors that express ENs in CD34÷ cells, we developed a miRNA-regulated system to suppress expression of the payload in 293-cells while allowing it in CD34+ cells. This enabled us to produce HD-Ad5/35 vectors expressing either a functionally active ZFN or a TALEN at high titers without vector genome rearrangements during production. We demonstrated that an HD-Ad5/35 vector expressing a CCR5 ZFN conferred the expected efficient knock-out in primitive human HSCs without affecting the viability and differentiation potential of these cells.

### Results

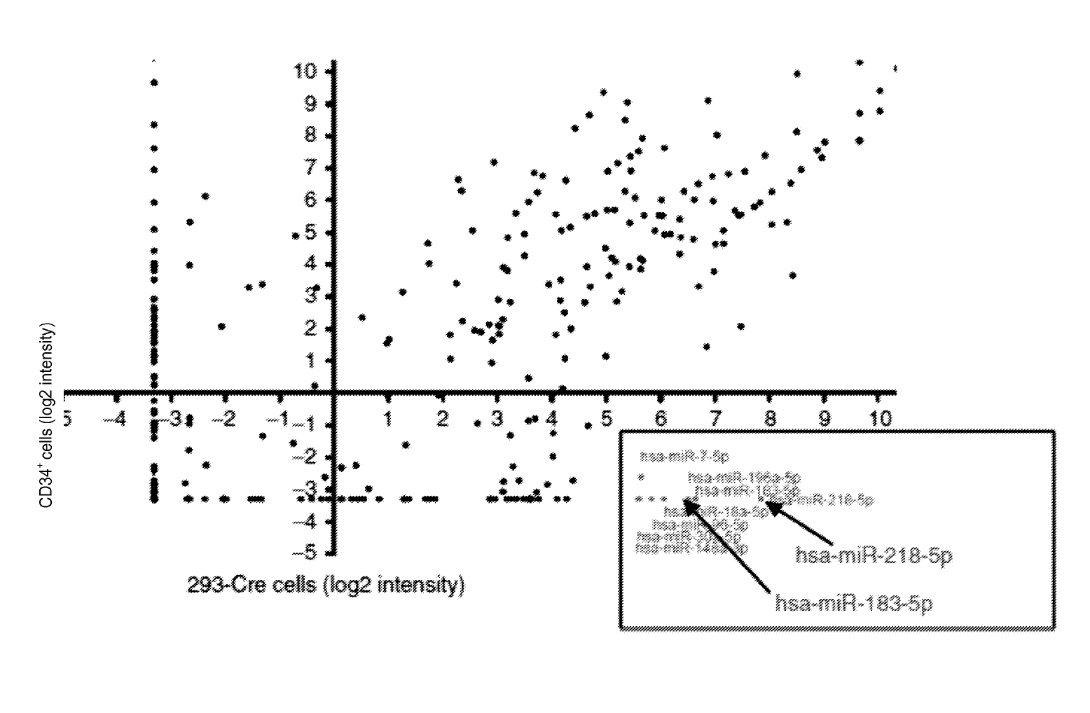

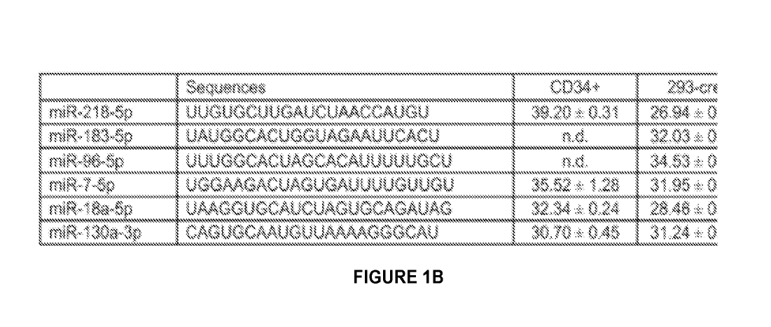

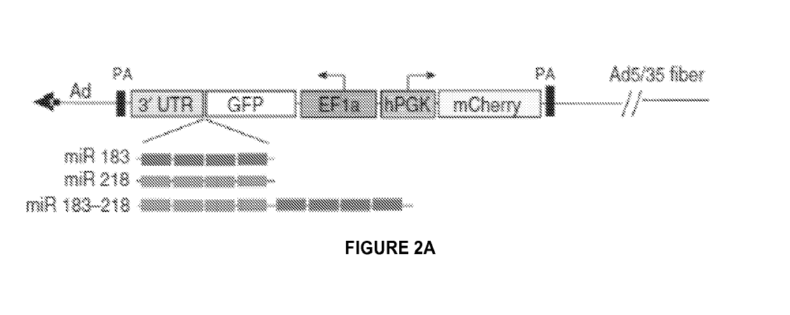

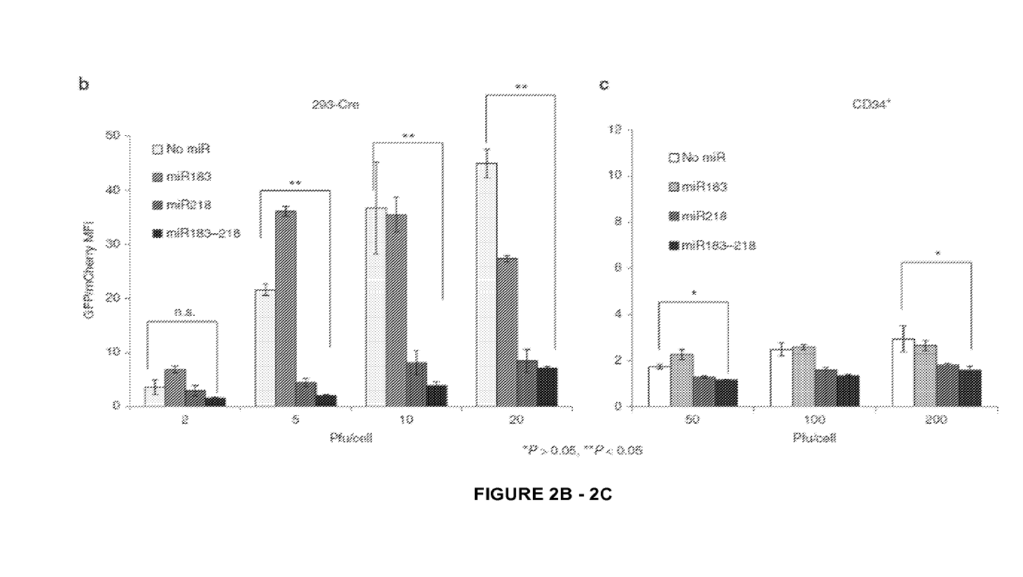

To generate HD-Ad5/35 vectors that express ZFN or TALEN transgenes in human hematopoietic CD34+ stem cells, we used a miRNA-regulated gene expression system. If the mRNA of a transgene contains a target site for a miRNA that is expressed at high levels in a given cell type, the mRNA will be degraded and transgene expression suppressed in this cell type. We set out to establish a miRNA-regulated expression system that would suppress transgene expression in HD-Ad producer cells, i.e. 293-Cre cells, while conferring it in our target cells, i.e. human CD34+ HSCs by establishing the miRNA expression profile in both cell types. Because Ad infection could interfere with the miRNA expression profile, we infected 293-Cre cells with Ad5/35 helper virus at an MOI of 20 pfu/cell, an MOI used for the amplification of HD-Ad vectors [28]. CD34+ cells front 4 different adult (GCSF-mobilized) donors were pooled and infected with an HD-Ad5/35 vector expressing GFP at an MOI of 2000 vp/cell, an MOI that confers efficient transduction of CD34+ cells [18]. Total RNA was purified 24 hours after Ad infection and hybridized onto array miRNA chips containing >2000 different human miRNAs probes (FIG. 1a). In total there were 8 candidate miRNAs (FIG. 1a) with high-level expression in 293-Cre cells, but absent or low expression in CD34+ cells. The expression levels of candidate miRNAs were measured by real-time PCR (FIG. 1b). Hsa-miR-7-5p and hsa-miR-18a-5p were removed front the candidate list, because they were also expressed in CD34+ cells at relatively high levels. miR-96-5p shared the same seed sequence at the 5′ end of the miRNA. (i.e. the sequence which critically determines miRNA target specificity [29]) with other miRNAs. Therefore, we did not include miR-96-5p into our selection. We then selected two miRNAs (hsa-miR-183-5p and hsa-miR-218-5p), which had the highest expression levels in 293-Cre cells and that were either undetectable (hsa-miR-183-5p) or expressed at the lowest detectable level (hsa-miR-218-5p) in CD34+ cells. To establish the miRNA-regulation system, we inserted 4 target sites with 100% homology to the selected two miRNAs alone and in combination into the 3′ untranslated region (UTR) of the globin gene. The UTR was linked to the 3′ end of a GFP gene, which was under the control of an EF1α promoter, a promoter that is highly active in CD34+ cells (FIG. 2a). The GFP expression cassettes were inserted into a first-generation Ad5/35 vector. The vectors also contained a PGK promoter-driven mCherry™ expression cassette that was not regulated by the selected miRNAs. Normalization of miRNA-regulated GFP expression to mCherry™ expression allows adjusting for differences in transduction efficiency between different vectors and cell types. We transduced CD34+ cells and 293-Cre cells with the vectors and analyzed GFP and mCherry™ expression 48 hours later by flow cytometry (FIGS. 2b and c). In 293-Cre cells, mCherry™ expression levels were comparable for all 4 vectors, while GFP expression was suppressed by vectors that contained the miRNA target sites. Based on the mean fluorescence intensity (MFI) ratio of GFP to mCherry™, the greatest suppression was achieved with the vector that contained both the target sequence of miR183 and miR218 (FIG. 2b). The difference in normalized GFP levels between the vector that contained the miR218 target sites only and the vector that contained the combination of both miR218 and miR183 target sites is significant and the p values decrease with increasing MOIs (MOI5: p=0.047, MOI10: p=0.033, MOI20: p=0.006) suggesting that miR183 target sites contribute to suppression of GFP expression.

Considering that first-generation Ad vectors replicate in 293-Cre cells and thus strongly express transgene products, the capability of the miR-183/218-based system to control GFP expression in 293-Cre cells is notable. This is further corroborated by the observation that normalized GFP levels do not increase in an MOI-dependent manner in 293-Cre cells. In contrast, in CD34+ cells both GFP and mCherry™ expression were comparably high for all vectors (FIG. 2c).

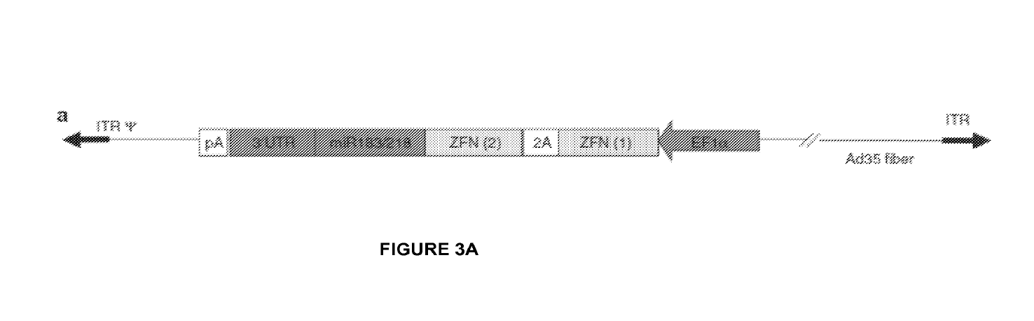

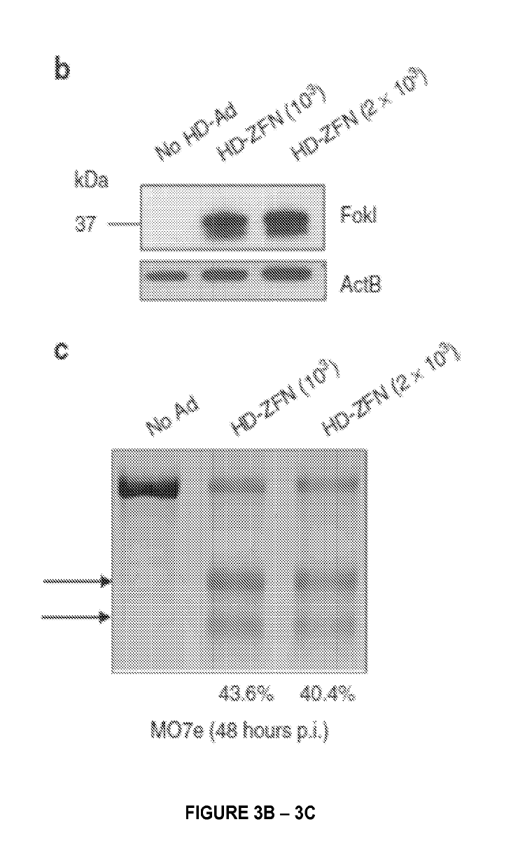

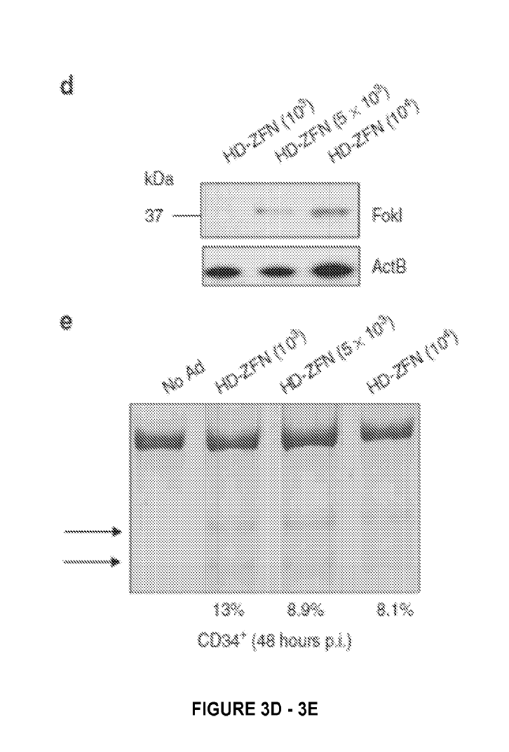

Using the miRNA-183/218 regulated gene expression system, we generated an HD-Ad5/35 vector expressing a ZFN under the control of the EF1α promoter (FIG. 3a). The ZFN was directed against the gene of the HIV co-receptor CCR5 [11]. The two ZFN subunits are linked through a self-cleaving picornavirus 2A peptide and are expressed as a poly-protein that is then cleaved. The miRNA-controlled ZFN expression cassette was inserted into a plasmid that, except the viral ITRs and packaging signal, lacked any sequences encoding for viral proteins [30]. The corresponding HD-Ad5/35.ZFNmiR vector (HD-ZFN) was produced in 293-Cre cells at high titers (1.88×10¹² vp/ml). Restriction analysis of viral DNA isolated from CsCl-gradient purified HD-ZFN particles did not reveal genomic rearrangements (Data not shown). To functionally test the HD-ZFN vector, we first performed transduction studies in MO7e cells, a CD34+ growth factor-dependent erythroleukemia cell line that is often used as a model for HSC gene therapy studies [31]. At day 2 post-transduction, half of the cells were used to analyze ZFN expression by Western blot using antibodies against the FokI domain (FIG. 3b). Genomic DNA was isolated from the other half of cells and analyzed for ZFN cleavage by T7E1 nuclease assay specific for the CCR5-ZFN target site (FIG. 3e). This analysis showed that HD-ZFN conferred site-specific DNA cleavage in >40% of ccr5 alleles in MO7e cells. An analogous study was then performed with human CD34+ cells from two different donors (donor A and donor B). Studies with cells from donor A are shown in FIGS. 3d and e. In Western blot analysis of cells collected 48 hours after infection, detectable FokI signals appeared when cells were infected at MOIs of equal or greater than 5×10³ vp/cell (FIG. 3d). Analysis of genomic DNA for ccr5 modification showed a disruption frequency of 13%, 8.9%, and 8.1% for MOIs of 10³, 5×10³, and 10⁴ vp/cell, respectively. Notably, the ccr5 disruption frequency did not increase with the MOI; it rather decreased most likely due to vector- or ZFN-related toxicity. Furthermore, gene disruption was seen in cells infected at an MOI of 10³ vp/cell, i.e. an MOI at which ZFN expression was below the Western blot detection level. The second study was performed with CD34+ cells from donor B. CD34+ cells from this donor were an aliquot from a CD34+ cell batch that was used for allogeneic HSC transplantation in cancer patients. The transduction efficacy with Ad5/35 and HD-Ad5/35 vectors was comparable to that of CD34+ cells from donor A. These cells can therefore be used to assess potential cytotoxicity of vector transduction. However, the genome of donor B cells contained a small nucleotide polymorphism within the ccr5 gene close to the ZFN cleavage site (Data not shown). The T7E1 nuclease assay is not able to distinguish between the SNP and ZFN-mediated rearrangements and therefore shows ccr5 disruption in all samples, including untransduced cells (Data not shown).

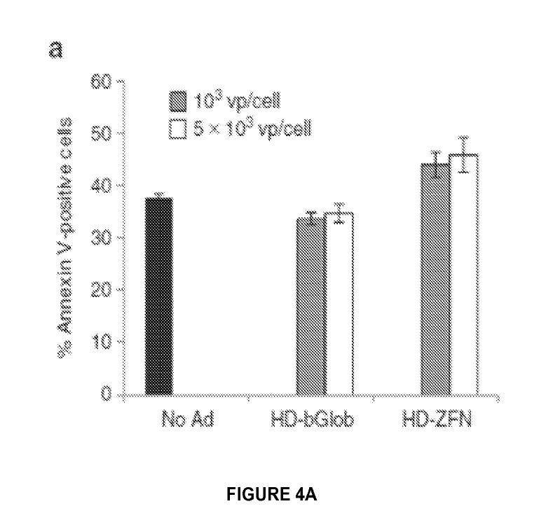

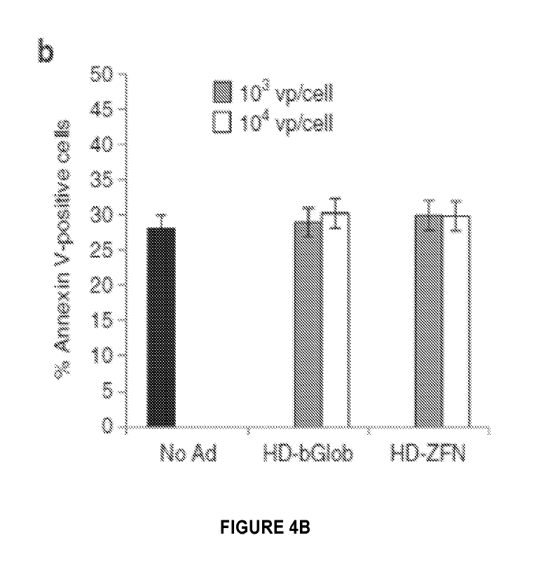

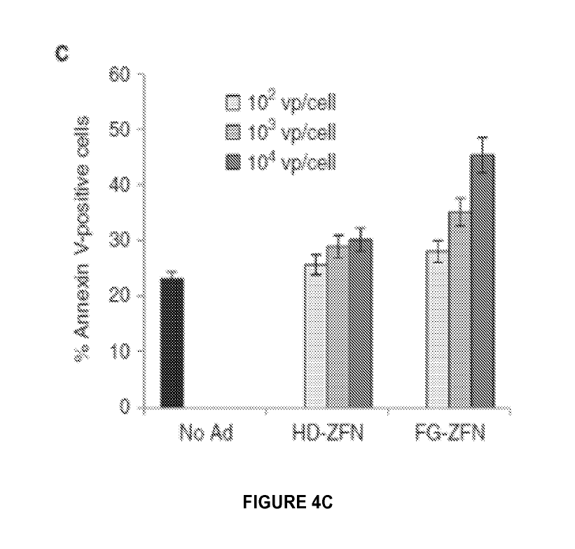

To assess whether ZFN expression from HD-Ad5/35 vectors causes cytotoxicity in CD34+ cells at the doses we used, we performed flow cytometry for the apoptosis marker Annexin V at day 4 after transduction. CD34+ cells used for this study were from donors A and B (FIGS. 4a and b). Although the outcome of the studies slightly differed between the two donors, HD-ZFN transduction did not significantly affect cell viability when compared to untransduced cells and control (HD-bGlob) vector transduced cells. In contrast, transduction of CD34+ with a first-generation Ad5/35 vector expressing the CCR5-ZFN [11] increased the percentage of Annexin V-positive cells in a dose-dependent manner in this experimental set up (FIG. 4c).

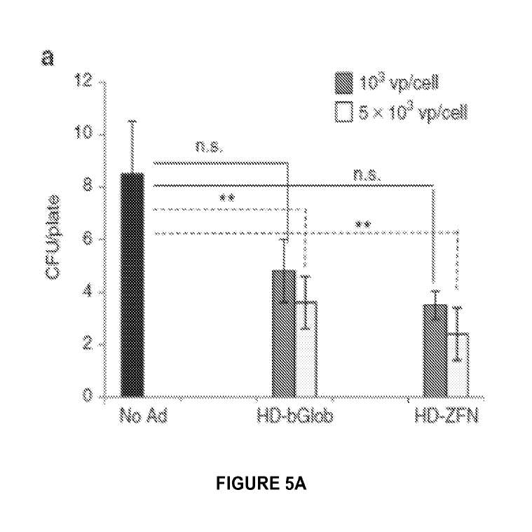

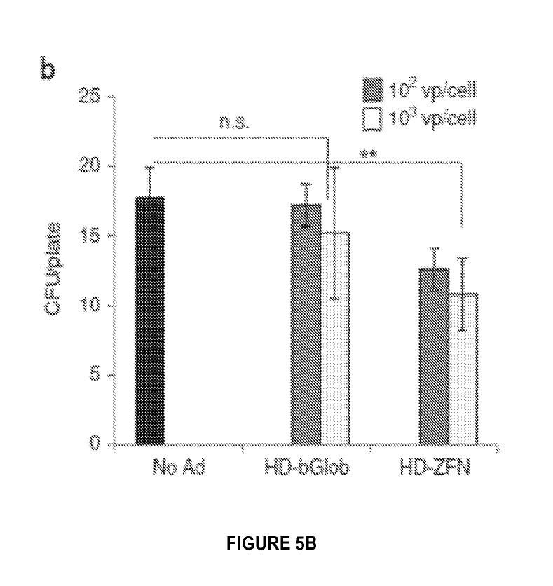

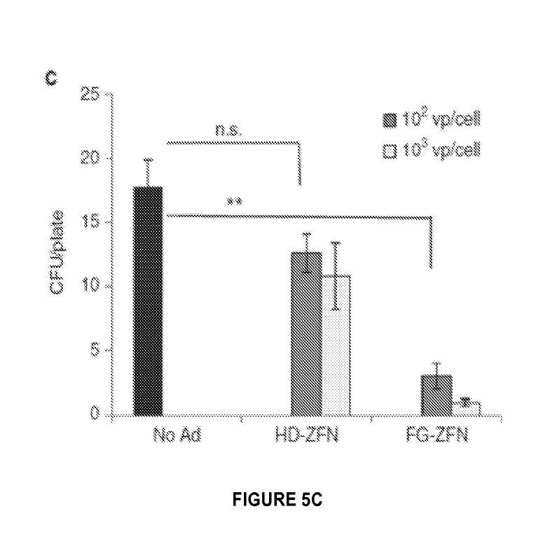

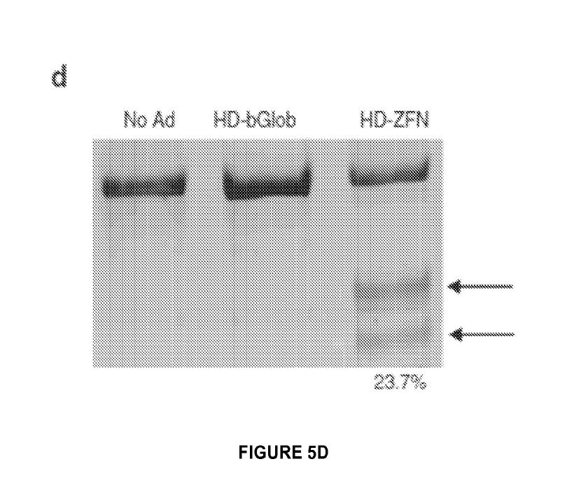

The next tasks were to show that HD-ZFN mediates CCR5 disruption in primitive HSCs and that transduction and ZFN expression do not affect the ability of these cells to proliferate and differentiate. To assess the latter, we subjected HD-ZFN-transduced CD34+ cells to a long-term culture initiating cell (LTC-IC) assay. This assay measures primitive HSCs based on their capacity to produce myeloid progeny for at least 5 weeks. Committed progenitors initially present in the transduced CD34+ cell population will rapidly mature and disappear during the initial 3 weeks of culture due to their limited proliferative potential. The more primitive cells will be maintained throughout the duration of culture and generate a new cohort of committed progenitors (e.g., colony-forming cells), which can be later detected and enumerated at the end of the assay using progenitor colony assays in semi-solid media. For both the control ID-bGlob and HD-ZFN vectors, transduction of CD34+ cells from donor A decreased the number of colonies compared to untransduced controls whereby the differences were significant only for MOI 5000 vp/cell (FIG. 5a). Transduction of CD34+ cells from donor B with the control vector did not significantly affect colony formation, while transduction with HD-ZFN at an MOI of 1000 vp/cell significantly decreased it (FIG. 5b). Transduction with FG-ZFN vector inhibited colony formation (FIG. 5c).

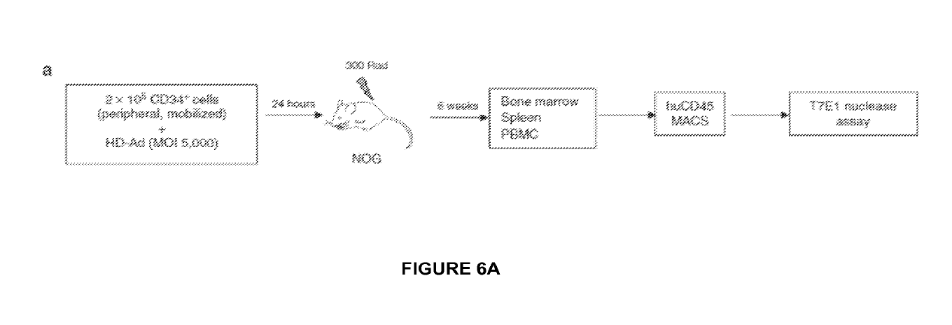

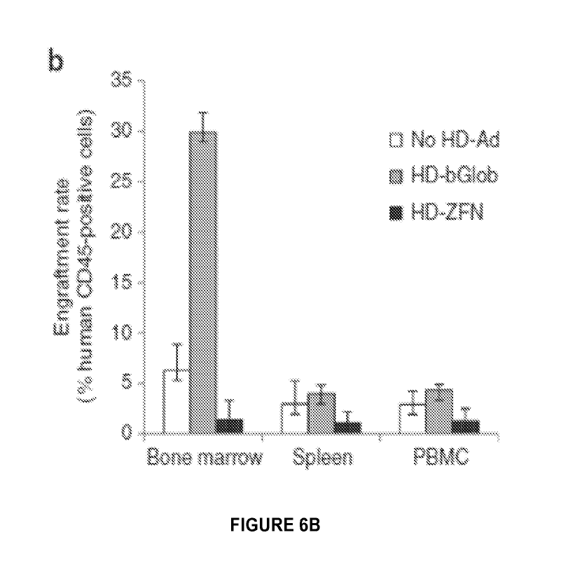

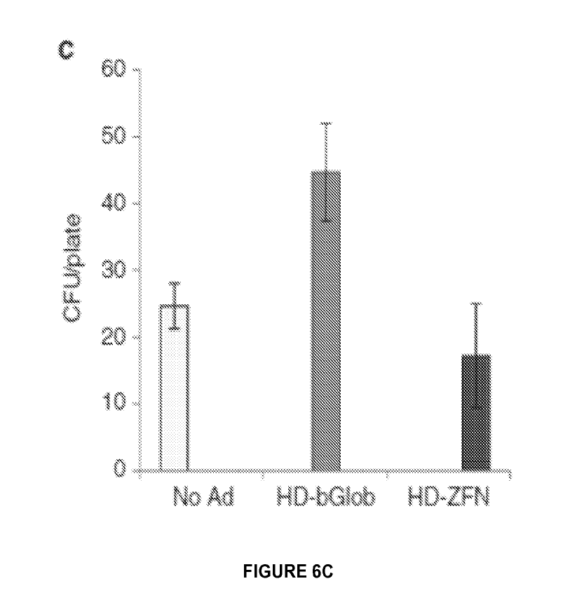

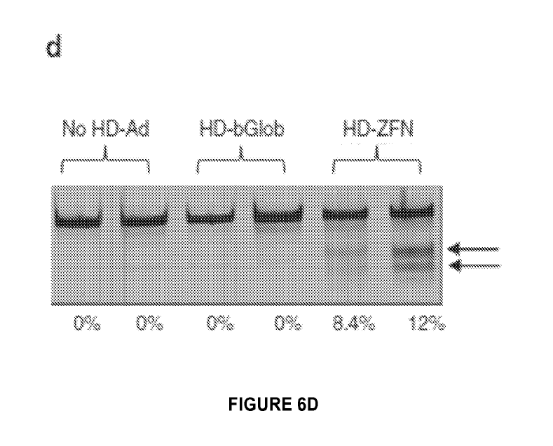

To evaluate CCR5 disruption levels in LTC-IC, cells from all colonies in a plate were combined, genomic DNA was isolated and subjected to T7E1 nuclease assay. The frequency of HD-ZFN-mediated ccr5 gene disruption in CFUs at the end of the assay was 23.7% (FIG. 5d). This suggested that the vector targeted primitive CD34+ cells and that the gene modification is persistent in HSC progeny. To further support this, we studied whether the HD-ZFN vector is able to mediate CCR5 disruption in NOD/SCID repopulating cells (FIG. 6a). This functional HSC assay is thought to potentially be predictive of the ability to repopulate conditioned recipients in human trials [32]. For this assay, we transduced CD34+ cells from donor A with the control HD-bGlob vector or HD-ZFN vector at an MOI of 5,000 vp/cell for 24 hours under low-cytokine conditions to prevent CD34+ cell differentiation. Transduced cells were transplanted into sublethally irradiated NOG mice. Engraftment of human cells was analyzed six weeks after transplantation by flow cytometry for human CD45+ cells in bone marrow, spleen and PBMC. (Notably, bone marrow engraftment rates with CD34+ cells from adult donors are usually lower than those achieved with umbilical cord-blood derived CD34+ cells). We found that 6% of bone marrow cells were human CD45+ positive in mice that were transplanted with non-transduced CD34+ cells (FIG. 6b). The average bone marrow engraftment rate of HD-ZFN transduced cells was 2.12%, which is about three fold lower than that of untransduced cells. Interestingly, transduction with the HD-b Glob vector increased the engraftment rate. Analysis of human CD45+ cells in the spleen and PBMC showed similar engraftment rates, although the effect of HD-bGlob transduction was less pronounced in these tissues. For further analyses, human CD45+ cells were purified using magnetic-activated cell sorting (MACS). Human CD45+ cells were subjected to progenitor/colony assays to assess the presence of HSCs (FIG. 6C). Similar numbers of colonies were found in engrafted CD45+ cells from mice that received untransduced or HD-ZFN transduced CD34+ cells. Colony numbers were higher for the HD-bGlob group suggesting that this vector improves the survival of HSCs. The reason for this remains elusive at this point. To investigate the frequency of CCR5 modification, human CD45+ cells were analyzed by T7E1 nuclease assay. We found the levels of ccr5 gene disruption to be 8.4% and 12% in two transplanted mice respectively (FIG. 6c). These data suggest that although HD-ZFN transduction and/or ZFN expression may decrease the engraftment rate of CD34+ cells, ccr5 gene disruption was achieved in HSCs that persisted in transplanted mice for the time of analysis.

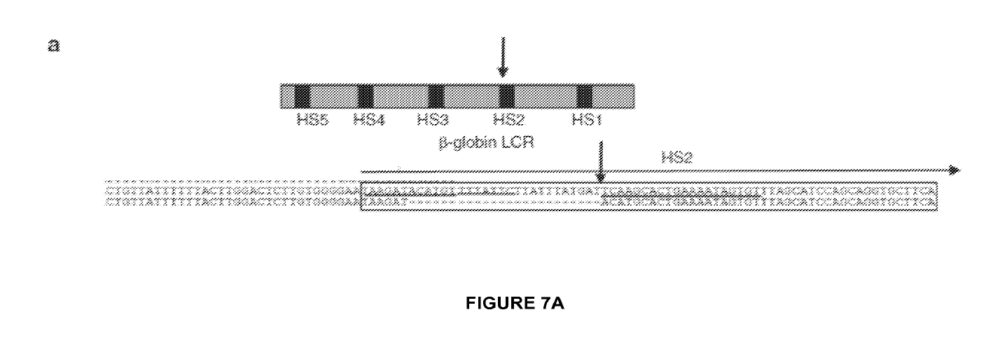

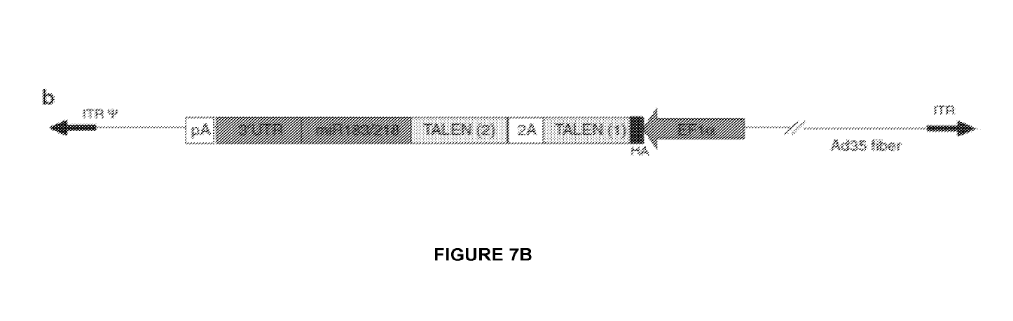

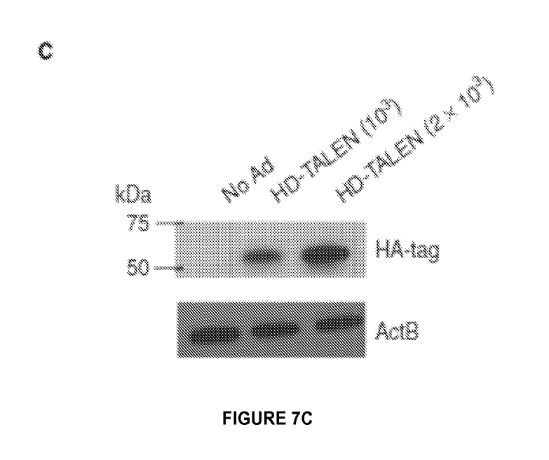

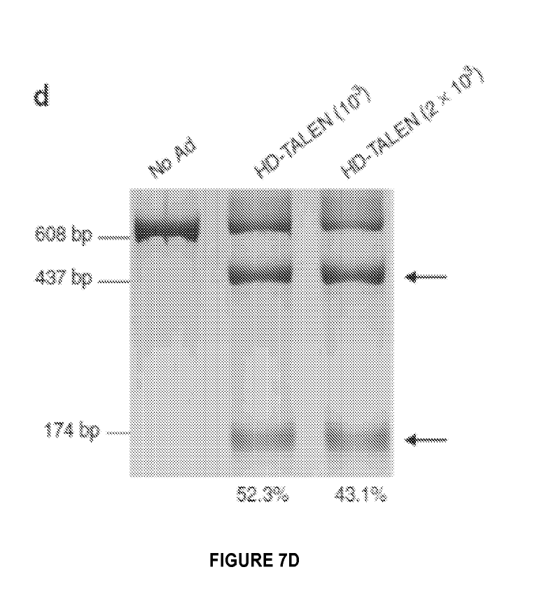

To show the versatility of our miRNA-based approach to regulate transgene expression, we produced a second vector expressing a TALEN targeting the DNase hypersensitivity region 2 (HS2) within the globin locus control region (LCR) (FIG. 7a). The site was selected because it is thought that target DNA sequences are better accessible to ENs when they are localized in active chromatin or DNase HS regions [33, 34]. We and others have shown in erythroid and hematopoietic stem cell lines that the HS2 region is occupied by open chromatin marks [35-38]. We have also previously shown that HD-Ad5/35 vectors carrying a 23-kb fragment of the β-globin locus control region preferentially integrated into the chromosomal β-globin LCR through chromatin tethering to the HS2 area [18, 35]. The latter studies were done in MO7e cells. As with ZFN-expressing HD-Ad5/35 vectors, our earlier attempts to rescue HD-Ad5/35-TALEN virus vectors (without mRNA-mediated suppression in 293 cells) were unsuccessful.

To generate the HD-Ad5/35.TALENmiR (HD-TALEN) vector, the 3′ end of the TALEN mRNA was modified to contain miR-183/218 binding sites (FIG. 7b). The HD-TALEN vector was produced at a high titer (2.5×10¹² VP/ml) without detectable genome rearrangements (FIG. 2b). After infection of MO7e cells with HD-TALEN at an MOI of 1000 vp/cell, TALEN expression was detected by Western blot using an anti-HA tag antibody (FIG. 7c). T7E1 nuclease assay revealed 50% modification of the HS2 target site in MO7e cells at day 2 after infection. The ability to place HS2 specific DNA breaks in combination with our globin LCR containing HD-Ad5/35 is relevant for targeted transgene insertion.

Taken together our studies show the miRNA system is a robust platform for the production of HD-Ad5/35 vectors expressing ZFNs and TALENs.

### Discussion

Because ZFNs were the first ENs developed, a substantial amount of data regarding site-specific and off-target activity has been accumulated for these types of ENs. A ZFN targeting the HIV CCR5 co-receptor gene was the first to be tested in clinical trials [12]. This trial involved the ex vivo transduction of patient CD4+ T-cells with a CCR5-ZFN expressing Ad5/35 vector. More recent efforts have focused on ccr5 gene knock-out in HSCs. Targeting HSCs vs CD4+ T cells has a number of advantages: i) As HSCs are a source for all blood cell lineages, CCR5 knockout would protect not only CD4 cells but also all remaining lymphoid and myeloid cell types that are potential targets for HIV infection. ii) in contrast to CD4+ cell transplants, a single HSC transplant would potentially provide a life-long source of HIV-resistant cells to allow long-term protection or control of HIV/AIDS. The first successful attempt to achieve ZFN-mediated disruption of ccr5 gene sequences in HSCs was reported by Holt et al. in 2010. This study demonstrated engraftment of the modified HSCs in NOD/SCID/IL2rγ^{null} (NSG) mice resulting in resistance to CCR5-tropic HIV-1 infection [3]. While encouraging, the data also indicated a number of potential problems, including the poor viability of cells transfected with the ZFN-expressing plasmid by electroporation in this experimental system.

To guard against the potential cytotoxicity of high level ZFN expression in 293-Cre cells in our system, we established a miRNA-based gene regulation system to suppress the ZFN transgene. The system is based on profiling of miRNA expression in 293-Cre cells and human CD34+ cells pooled from different donors. Studies with reporter genes showed efficient suppression of a transgene that was regulated by hsa-miR-183-5p and hsa-miR-218-5p. While there was background expression of the miRNA-regulated GFP reporter gene, it did not increase in a dose-dependent manner or upon viral replication. The latter could be due to the high levels of miR-183 and -218 in 293-Cre cells and complete saturation of the corresponding target sites. Importantly, the miR183/218-regulation system was successful for the generation of HD-Ad5/35 vectors expressing the CCR5 ZFN or the globin LCR TALEN. Potentially, our miRNA-regulated approach is also relevant for the production of lentivirus or rAAV vectors which also use 293 cells as production cells.

In transduction studies we focused on HD-Ad5/35.ZFNmiR (HD-ZFN). ZFN expression analyzed at day 2 after infection was lower in CD34+ cells than in MO7e cells. This is in agreement with our previous studies with HD-Ad5/35.GFP vectors where we showed that transduction of CD34+ cells results in GFP expression in ˜60% of CD34+ cells and mean GFP fluorescence intensity levels that were about ˜10 fold lower than in MO7e cells. Analysis of ccr5 gene disruption at day 2 after HD-ZFN transduction did not show a correlation with ZFN expression level at this time point. Analysis at a later time point following transduction potentially would show a higher level of disruption. It is possible that cellular factors, specifically proteins involved in non-homologous end joining (NHEJ) DNA repair limit the disruption efficiency rearrangement efficacy. Alternatively, considering that CD34+ cells is a highly heterogeneous cell population, it is possible that HD-Ad5/35 transduction, ZFN cleavage, and/or NHEJ occurs only in fraction of CD34+ cells. Importantly our subsequent LTC-IC and NOG mice repopulation studies suggested that the targeted CD34+ cells contain primitive stem cells.

We found that HD-ZFN transduction decreased the engraftment rate, survival and/or expansion of CD34+ cells in NOG mice in our system. This was not necessarily due to HD-Ad/35 transduction and vector-associated toxicity per se, because engraftment rates were actually higher with HD-bGlob transduced CD34+ cells than with non-transduced cells. We therefore speculate that this is related to ZFN expression over an extended time period. Non-integrating HD-Ad vector genomes are lost after several rounds of cell division, however persist longer in non-dividing cells such as hepatocytes [43]. Because HSCs are low proliferative, HD-Ad5/35 genomes could be maintained for longer time periods and thus express ZFN. For gene engineering purposes, it is sufficient that ZFNs are expressed only for a short time period.

It is noteworthy that we used in our studies CD34+ cells from adult G-CSF mobilized donors, a source that is more readily available than fetal liver or cord blood derived CD34+ cells, which were used in previous studies with CCR5 ZFNs [2, 3]. A ccr5 gene disruption frequency of 12% in engrafted HSCs found in the bone marrow of transplanted NOG mice is clinically relevant for HIV therapy considering that these cells can give rise to multiple lineages, including lineages that represent targets and reservoirs for HIV.

Another avenue that we are following is to use the globin LCR-specific TALEN to increase the site-specific integration of a donor HD-Ad5/35 vector through homologous recombination [18].

The HD-Ad5/35 vector platform of the present invention for EN gene delivery to HSCs has major advantages over other delivery systems, i) Most importantly it allows for efficient targeting of primitive HSCs with less cytotoxicity. ii) The insert capacity of HD-Ad vectors is 30 kb which allows the accommodation of several ENs and homologous donor templates. This is important for the simultaneous editing of multiple genes in HSCs for gene therapy purposes or to establish relevant models for multigenic human diseases. The use of HD-ADS/35 vector would also make it possible to combine both the EN expression cassette and the donor DNA sequences with extended homology regions into one vector. In this context is notable that the efficacy of homologous recombination directly correlates with the length of the homology regions [16]. iii) HD-Ad vectors allow for the transduction of target cells in vivo. HD-Ad5 vectors efficiently transduce hepatocytes in mice and non-human primates after intravenous injection [44, 45]. Our preliminary studies in human CD34+/NOG and human CD46-transgenic mice show that affinity-enhanced Ad5/35 and HD-Ad5/35 vectors can transduce GCSF/AMD3100 mobilized HSCs after intravenous injection [22]. HSC gene editing approaches involving the in vitro culture/transduction, and retransplantation into myelo-conditioned patients are technically complex and expensive. The in vitro culture of HSC in the presence of multiple cytokines affects the viability, pluripotency and engraftment potency of HSCs. Furthermore, the need for myeloablative regimens creates additional risks for patients. Finally, the procedure is expensive and can only be performed in specialized institutions. Therefore vectors system that allow for in vivo HSC genome editing are of relevance.

In summary, we have developed a miRNA-regulated HD-Ad5/35 vector platform for the expression of designed endonucleases in primitive HSCs. This vector system is a new important tool for genome engineering of HSCs for therapeutic purposes.

### Materials and Methods

Cells: 293 cells, 293-C7-CRE [46] cells were cultured in Dulbecco's modified Eagle's medium (Invitrogen,) supplemented with 10% fetal calf serum (FCS) (HyClone™), 2 mM L-glutamine, Pen-Strep. Mo7e cells [31] were maintained in RPMI 1640 medium containing 10% FCS, 2 mM L-glutamine, Pen-Strep, and granulocyte-macrophage colony stimulating factor (0.1 ng/ml) (Peprotech™). Primary human CD34+-enriched cells from G-CSF mobilized normal donors were obtained from the Fred Hutchinson Cancer Research Center Cell Processing Core Facility. We used CD34+ cells from two different donors, designed “donor A” and “donor B”. CD34+ cells were recovered from frozen stocks and incubated overnight in Iscove's modified Dulbecco's medium (IMDM) supplemented with 20% FCS, 0.1 mM 2-mercaptoethanol, stem cell factor (50 ng/ml), DNase I (100 μg/ml), 2 mM L-glutamine, Flt3 ligand (Flt3L, 50 ng/ml), interleukin (IL)-3 (10 U/ml), and thrombopoietin (10 ng/ml). Cytokines and growth factors were from Peptotech.

micro-RNA array: Array studies were performed using Agilent's human miRNA (8×60 K) V18.0 containing 2006 different human miRNAs probes. Extraction of miRNA and RNA from Qiagen RNAprotect™ cell reagent stabilized cells was performed according to the Qiagen miRNeasy™ kit protocol. RNA samples were frozen at −80° C. Each slide was hybridized with 100 ng Cy3-labeled RNA using miRNA Complete Labeling and Hyb Kit (Agilent Technologies) in a hybridization oven at 55° C., 20 rpm for 20 hours according to the manufacturer's instructions. After hybridization, slides were washed with Gene Expression Wash Buffer Kit (Agilent). Slides were scanned by Agilent Microarray Scanner and Feature Extraction software 10.7 with default settings. Raw data were normalized by Quantile algorithm, Gene Spring Software 11.0.

qRT-PCR for selected miRNAs. RNA prep concentration was measured using ScanDrop™ (Analytik Jena, Germany). The reverse transcription was performed using TaqMan™ miRNA Reverse Transcription Kit with miRNA specific primers all purchased from Applied Biosystems, using 5 ng template, 4° C. 6 min, 16° C. 30 min, 42° C. 30 min, and 85° C. 5 min. The Real-Time PCR was performed in quadruplicate with TaqMan 2× Universal PCR Master Mix with no AmpErase™ UNG on a 7900HT machine (Applied Biosystems), using 0.27 ng template in 10 μl reaction volume, 95° C. for 10 minutes, 40 cycles of 95° C. for 15 seconds, 60° C. for 60 seconds. The Ct value was calculated at threshold equals 0.3, and with manual baseline start cycle at 3 and end cycle at 13. miRNA homology in the 5′ seed sequences was analysed using “R software” and “microRNA” bioconductor package [29].

### Adenovirus Vectors

Ad5/35-RG containing miRNA target sites: The GFP-mCherry™ cassette from pRG₀ [47] was transferred into the adenovirus shuttle plasmid pDeltaE1/Sp1 (Microbix). The following miRNA target sites were synthesized and inserted into the AvrII/SmaI site of the shuttle vectors:

miR-183 target site:
(SEQ ID NO: 147)
5′CTAGGATTATGGCACTGGTAGAATTCACTACTTATGGCACTGGTAGAA
TTCACTACTTATGGCACTGGTAGAATTCACTACTTATGGCACTGGTAGAA
TTCACTATCGCCCGGG
miR-218 target site:
(SEQ ID NO: 148)
5′CCTAGGAATTTGTGCTTGATCTAACCATGTTTCATTGTGCTTGATCTA
ACCATGTTTCATTGTGCTTGATCTAACCATGTTTCATTGTGCTTGATCTA
ACCATGTATCGCCCGGG
miR-183/218 target site:
(SEQ ID NO: 149)
5′CCTAGGAT TATGGCACTGGTAGAATTCACT ACT
TATGGCACTGGTAGAATTCACT ACT TATGGCACTGGTAGAATTCAT
ACT
TATGGCACTGGTAGAATTCACT ATCG TTGTGCTTGATCTAACCATGT
TTCAT
TGTGCTTGATCTAACCATGT TTCAT TGTGCTTGATCTAACCATGT
TTCATTGTGCTTGATCTAACCATGT ATCGCCCGGG

First-generation Ad5/35 virus vectors were generated and tested as described elsewhere [6].

### HD-Ad5/35-ZFN Containing the miR-182/219-Regulated CCR5 ZFN Under EF1a Promoter Control

The shuttle plasmid for recombination in HD backbone vector was generated using pBluescript™ (pBS) plasmid. Briefly recombination arms were amplified from pHCA plasmid containing stuffer DNA [30] and cloned into pBS generating pBS -7 for ZFN-construct and pBS-T for Talen-LCR construct. 3′UTR and pA sequence was synthesized by Genescript and cloned into both shuttle vectors via AgeI and XhoI generating pBS-Z-3′UTR-pA and pBS-T-3′UTR-pA. Ef1a promotor was extracted from PJ204-EF1a-pA containing a 1335 bp fragment of the EF1a promoter with BamHI and NheI, then inserted into respective sites in both shuttle plasmids generating pBS-Z-Ef1a and pBS-T-EF1a. ZFN-CCR5 fragment from pBS-CCR5 [11] was digested with EcoRI and XbaI and cloned into the shuttle vector generating pBS-Ef1a-ZFN-CCR5. Finally synthesized miR-183/218 tandem repeats flanked by NotI were cloned into its respective site in pBS-Ef1a-ZFN-CCR5 generating pBS-Ef1a-ZFN-CCR5-miR. The shuttle vector plasmids were linearized with BstBI and recombined with pHCA backbone vector in E. coli BJ5183 cells. Recombined pHCA-Ef1a-ZFN-CCR5-miR and pHCA-Ef1a-Talen-LCR-miR were then linearized with PmeI and rescued in 293-Cre cells with helper virus (HV-AD5/35) to generate HD-Ad5/35-EF1a-ZFN-CCR5-miR virus (HD-Ad5/35.ZFNmiR) and HD-Ad5/35-EF1a-Talen-LCR-miR virus (HD-Ad5/35Talen.miR).

### HD-Ad5/35-TALEN Containing the miR-182/219-Regulated HS2-LCR TALEN Under EF1a Promoter Control

The HS2-LCR-specific TALEN was designed by ToolGen™ (Seoul, South Korea) as described previously [48]. The TALEN recognition sequences are shown in FIG. 7a. The DNA binding domains are fused with FokI. The N-terminus of the DNA binding domain is tagged with a hemagglutinin (HA)-tag and contains a nuclear localization signal. The TALEN cassette was under the control of the EF1a promoter and contained miR sites upstream of 3′UTR. The two TALEN were cloned into PBS-T-EF1a and linked via 2A peptide. Similar to the ZFN-CCR5 construct miR 183/218 tandem repeats were synthesized and cloned into NotI site of pBS-EF1a-Talen-LCR generating pBS-EF1a-Talen-LCR-miR. For virus rescue the final plasmid was linearized with PmeI.

HD-Ad5/35.bGlob (HD-bGlob). This vector has been described previously [18]. It contains ˜26 kb of the globin LCR. The β-globin promoter controls the expression of a GFP gene. HD-Ad5/35 vectors were produced in 293-Cre cells [28] with the helper virus Ad5/35-helper [42] as described in detail elsewhere [28]. Helper virus contamination levels were determined as described elsewhere and were found to be <0.05%. DNA analyses of HDAd genomic structure were confirmed as described elsewhere [28].

Flow cytometric analysis. For cytotoxicity analysis, Ad transduced CD34+ cells were stained with the AnnexinV/7AAD apoptosis kit (eBiosciences). For engraftment analysis cells derived from PBMCs, bone marrow and spleen were stained with anti hCD45-PE (BD). The data was then analyzed with FlowJo™ software.

Magnetic-activated cell sorting (MACS). Anti-human CD45 conjugated microbeads were from Miltenyi Biotech. Cell purification was performed according to the manufacturer's protocol.

LTC-IC (Long term culture-initiating cell) assay: Transduced CD34+ cells were incubated in cytokine containing IMDM for 48 hours after which they were transferred to long-term initiating culture conditions. Briefly, adherent murine bone M2-10B4 Fibroblast feeder cell layers were established as described by StemCell Technologies. Transduced CD34+ cells were added to the feeder layer and incubated for 5 weeks in human long term initiating culture medium with 10⁻⁶ M Hydrocortisone (HLTM) (StemCell Technologies), with weekly half medium changes. After 5 weeks cells were collected and subjected to colony forming unit assay.

Colony forming unit assays: For colony forming unit assay, 2×10⁴ cells were transferred from LTC-IC into MethoCult™ GF H4434 medium (StemCell Technologies) in a humidified atmosphere of 5% CO₂ at 37° C. in the presence of the following cytokines: (IL-3 50 U/ml, SCF 50 ng/ml, Epo 2 U/ml, G-CSF 6.36 ng

Western blot: Cell pellets in ice-cold PBS containing protease inhibitors (Complete Protease Inhibitor Cocktail, Roche) were sonicated and the protein containing supernatant stored at −80° C. A total of 20 μg of total protein was used for the Western blot analysis, Proteins were separated by polyacrylamide gel electrophoresis (PAGE) using 4-15% gradient gels (BioRad), followed by transfer onto nitrocellulose membranes according to the supplier's protocol (Mini ProteanIII, BioRad). Membranes were blocked in 5% non-fat dry milk (Bio-Rad) and washed in Tris-saline with 0.1% Tween-20 (TBS-T). Membranes were incubated with anti-FokI antibody (Sangamo BioSciences), anti-HA tag (Roche), or anti-beta-actin (Sigma Aldrich). Membranes were developed with ECL plus reagent (Amersham).

Mismatch sensitive nuclease assay T7E1 assay. Genomic DNA was isolated as previously described [49]. CCR5 or LCR region was amplified. Primers for detection of CCR5 disruption were described previously [50]. Primers for HS-LCR site analysis were: 5′AAATCTTGACCATTCTCCACTCTC (SEQ ID NO: 150) and 5′GGAGACACACAGAAATGTAACAGG (SEQ ID NO: 151). PCR products were hybridized and treated with 2.5 Units of T7E1 (NEB). Digested PCR products were resolved by 10% TBE PAGE (Biorad) and stained with ethidium bromide. Band intensity was analyzed using ImageQuant™ software.

Animal studies: All experiments involving animals were conducted in accordance with the institutional guidelines set forth by the University of Washington. Mice were housed in specific-pathogen-free facilities. The immunodeficient NOG mice strain name: NOD/Shi-scid/IL-2Rγnull) were obtained from the Jackson Laboratory.

CD34+ cell transplantation: Cryo-conserved CD34+ cells were thawed in PBS supplemented with 1% heat inactivated FCS. Freshly thawed cells were cultured overnight in IMDM containing 10% heat inactivated FCS, 10% BSA, 4 mM Glutamine and Penicillin/Streptomycin, as well as human cytokines (TPO (5 ng/mL), SCF (25 ng/mL), IL-3 (20 ng/mL), Flt3L (50 ng/mL)). The next day cells were infected with HD-bGlob or HD-ZFN at an MOI of 5000 vp/cell and incubated for 24 h. Uninfected cells were used as control. The next day, NOG recipient mice received 300 Rad/3 Gy total body irradiation. 24 h post infection 3×10⁵ transduced CD34+ cells were mixed with 2.5×10⁵ freshly collected bone marrow cells of non-irradiated NOG mice and injected i.v. into recipient mice at 4 hours post irradiation. Six weeks after bone marrow transplantation the engraftment rate was assayed as follows: blood samples were drawn, red blood cells were lysed and the remaining cells were stained with PE-conjugated anti human CD45 antibodies and analyzed via flow cytometry. 6 weeks after transplantation bone marrow cells were subjected to double sorting with anti hCD45 (Miltenyi) beads and seeded on methylcellulose. After two weeks colonies were counted and subjected to T7E1 nuclease assay.

### REFERENCES

1. Kim H, Kim J S (2014). A guide to genome engineering with programmable nucleases. Nat Rev Genet 15: 321-334.

2. Genovese P, et al. (2014). Targeted genome editing in human repopulating haematopoietic stem cells. Nature 510: 235-240.

3. Holt N, et al. (2010). Human hematopoietic stein/progenitor cells modified by zinc-finger nucleases targeted to CCR5 control HIV-1 in vivo. Nat Biotechnol 28: 839-847.

4. Pelascini L P, Goncalves M A (2014). Lentiviral vectors encoding zinc-finger nucleases specific for the model target locus HPRT1. Methods Mol Biol 1114: 181-199.

5. Pelascini L P, Maggio I, Liu J, Holkers M, Cathomen T, Goncalves M A (2013). Histone deacetylase inhibition rescues gene knockout levels achieved with integrase-defective lentiviral vectors encoding zinc-finger nucleases. Hum Gene Ther Methods 24: 399-411.

6. Shayakhmetov D M, Papayannopoulou T, Stamatoyannopoulos G, Lieber A (2000). Efficient gene transfer into human CD34(+) cells by a retargeted adenovirus vector. Virol 74: 2567-2583.

7. Li L, et al. (2013). Genomic editing of the HIV-1 coreceptor CCR5 in adult hematopoietic stem and progenitor cells using zinc finger nucleases. Mol Ther 21: 1259-1269.

8. Yotrida P, et al. (2001). Efficient infection of primitive hematopoietic stein cells by modified adenovirus. Gene Ther 8: 930-937.

9. Lu Z Z, et al. (2006). Efficient gene transfer into hematopoietic cells by a retargeting adenoviral vector system with a chimeric fiber of adenovirus serotype 5 and 11p. Exp Hematol 34: 1171-1182.

10. Nilsson M, Karlsson S, Fan X (2004). Functionally distinct subpopulations of cord blood CD34+ cells are transduced by adenoviral vectors with serotype 5 or 35 tropism. Mol Ther 9: 377-388.

11. Perez E E, et al. (2008). Establishment of HIV-1 resistance in T cells by genome editing using zinc-finger nucleases. Nat Biotechnol 26: 808-816.

12. Tebas P, et al. (2014). Gene editing of CCR5 in autologous CD4 T cells of persons infected with HIV. N Engl J Med 370: 901-910.

13. Lieber A, He C Y, Kirillova I, Kay M A (1996). Recombinant adenoviruses with large deletions generated by Cre-mediated excision exhibit different biological properties compared with first-generation vectors in vitro and in vivo J Virol 70: 8944-8960.

14. Shimizu K, Sakurai F, Machitani M, Katayama K, Mizuguchi H (2011). Quantitative analysis of the leaky expression of adenovirus genes in cells transduced with a replication-incompetent adenovirus vector. Mol Pharm 8: 1430-1435.

15. Morral N, et al. (1998). High doses of a helper-dependent adenoviral vector supraphysiological levels of alpha 1-antitrypsin with negligible toxicity. Hum Gene Ther 9: 2709-2716.

16. Balamotis M A, Huang K, Mitani K (2004). Efficient delivery and stable gene expression in a hematopoietic cell line using a chimeric serotype 35 fiber pseudotyped helper-dependent adenoviral vector. Virology 324: 229-237.

17. Wang H, Cao H, Wohlfahrt M, Kiem H P, Lieber A (2008). Tightly regulated gene expression in human hematopoietic stem cells after transduction with helper-dependent Ad5/35 vectors. Experimental Hematology in press.

18. Wang H, et al. (2005). A capsid-modified helper-dependent adenovirus vector containing the beta-globin locus control region displays a nonrandom integration pattern and allows stable, erythroid-specific gene expression. J Viral 79: 10999-11013.

19. Kochanek S, Schiedner G, Volpers C (2001). High-capacity ‘gutless’ adenoviral vectors. Curr Opin Mol Ther 3: 454-463.

20. Wang H, Cao H, Wohlfahrt M, Kiem H P, Lieber A (2008). Tightly regulate(gene expression in human hematopoietic stem cells after transduction with helper-dependent Ad5/35 vectors. Exp Hematol 36: 823-831.

21. Wang H, Lieber A (2006). A helper-dependent capsid-modified adenovirus vector expressing adeno-associated virus rep78 mediates site-specific integration of a 27-kilobase transgene cassette. J Virol 80: 11699-11709.

22. Richter M, et al. (2014). in vivo transduction of mobilized hematopoietic stem cells with an affinity-enhanced Ad5/35 vector. Molecular Therapy 22.

23. Holkers M, et al. (2013). Differential integrity of TALE nuclease genes following adenoviral and lentiviral vector gene transfer into human cells. Nucleic Acids Res 41: e63.

24. van Renshurg R. et al. (2013). Chromatin structure of two genomic sites for targeted transgene integration in induced pluripotent stem cells and hematopoietic stem cells. Gene Ther 20: 201-214.

25. Brown B D, Venneri M A, Zingale A, Sergi Sergi L, Naldini L (2006). Endogenous microRNA regulation suppresses transgene expression in hematopoietic lineages and enables stable gene transfer. Nat Med 12: 585-591.

26. Mullokandov G, et al. (2012). High-throughput assessment of microRNA activity and function using microRNA sensor and decoy libraries. Nat Methods 9: 840-846.

27. Brown B D, Naldini L (2009). Exploiting and antagonizing microRNA regulation for therapeutic and experimental applications. Nat Rev Genet 10: 578-585.

28. Palmer D J, Ng P (2005). Helper-dependent adenoviral vectors for gene therapy. Hum Gene Ther 16: 1-16.

29. Wang X (2014). Composition of seed sequence is a major determinant of microRNA targeting patterns. Bioinformatics 30: 1377-1383.

30. Sandig V. et al. (2000). Optimization of the helper-dependent adenovirus system for production and potency in vivo. Proc Natl Acad Sci U S A 97: 1002-1007.

31. Avanzi G C, et al. (1988). Selective growth response to IL-3 of a human leukaemic cell line with megakaryoblastic features, Br J Haematol 69: 359-366.

32. Dick J E, Bhatia M, Gan O, Kapp U, Wang J C (1997). Assay of human stem cells by repopulation of NOD/SCID mice. Stem Cells 15 Suppl 1: 199-203; discussion 204-197.

33. Kuscu C, Arslan S, Singh R, Thorpe J, Adli M (2014). Genome-wide analysis reveals characteristics of off-target sites bound by the Cas9 endonuclease. Nat Biotechnol.

34. Wu X, et al. (2014). Genome-wide binding of the CRISPR endonuclease Cas9 in mammalian cells. Nat Biotechnol.

35. Sova P, Wang H, Bomsztyk K, Stamatoyannopoulos G, Lieber A (2008). Role of chromatin structure in integration of helper-dependent adenoviral vectors containing the beta-globin locus control region, Hum Gene Ther 19: 153-166.

36. Fang X, Yin W, Xiang P, Han H, Stamatoyannopoulos G, Li Q (2009). The higher structure of chromatin in the LCR of the beta-globin locus changes during development. J Mol Biol 394: 197-208.

37. Kim A, Kiefer C M, Dean A (2007). Distinctive signatures of histone methylation in transcribed coding and noncoding human beta-globin sequences. Mol Cell Biol 27: 1271-1279.

38. Kooren J, et al. (2007), beta-globin Active Chromatin Hub formation in differentiating erythroid cells and in p45 NF-E2 knockout mice. J Biol Chem.

39. Kohn D B, Pai S Y, Sadelain M (2013). Gene therapy through autologous transplantation of gene-modified hematopoietic stem cells. Biol Blood Marrow Transplant 19: S64-69.

40. van der Vliet (1995), Adenovirus DNA replication, in: W. Doerfler aPB (ed). The molecular repertoire of adenoviruses, vol. 2. Springer Verlag: Berlin. pp 1-31.

41. Steinwaerder D S, Carlson C A, Lieber A (1999). Generation of adenovirus vectors devoid of all viral genes by recombination between inverted repeats. J Virol 73: 9303-9313.

42. Shayakhmetov D M, et al. (2004). Genuine size and structure determine efficiency of postinternalization steps and gene transfer of capsid-modified adenovirus vectors in a cell-type-specific manner. J Virol 78: 10009-10022.

43. Ehrhardt A, Xu H, Kay M A (2003). Episomal persistence of recombinant adenoviral vector genomes during the cell cycle in vivo. J Virol 77: 7689-7695.

44. Brunetti-Pierri N, et al. (2013). Transgene expression up to 7 years in nonhuman primates following hepatic transduction with helper-dependent adenoviral vectors. Hum Gene Ther 24: 761-765.

45. Kubo S, et al. (2010). In vivo stable transduction of humanized liver tissue in chimeric mice via high-capacity adenovirus-lentivirus hybrid vector. Hum Gene Ther 21: 40-50.

46. Palmer D, Ng P (2003). Improved system for helper-dependent adenoviral vector production. Mol Ther 8: 846-852.

47. Papapetrou E P, Kovalovsky D, Beloeil L, Sant'angelo D, Sadelain M (2009). Harnessing endogenous miR-181a to segregate transgenic antigen receptor expression in developing versus post-thymic T cells in murine hematopoietic chimeras. J Clin Invest 119: 157-168.

48. Kim Y, et al. (2013). A library of TAL effector nucleases spanning the human genome. Nat Biotechnol 31: 251-258.

49. Miller J C, et al. (2007). An improved zinc-finger nuclease architecture for highly specific genome editing. Nat Biotechnol 25: 778-785.

50. Perez L E, Rinder H M, Wang C, Tracey J B, Maun N, Krause D S (2001). Xenotransplantation of immunodeficient mice with mobilized human blood CD34+ cells provides an in vivo model for human megakaryocytopoiesis and platelet production. Blood 97: 1635-1643.

### EXAMPLE 2

CCR5 directed AIDS therapy: There are two co-receptors of CD4 for HIV infection, CCR5 and CXCR4. HIV isolated from infected individuals early after infection are predominantly CCR5-tropic, indicating a key role of CCR5 in the initial infection with HIV. This is supported by the fact that individuals with a homozygous deletion in the ccr5 gene are protected against HIV.

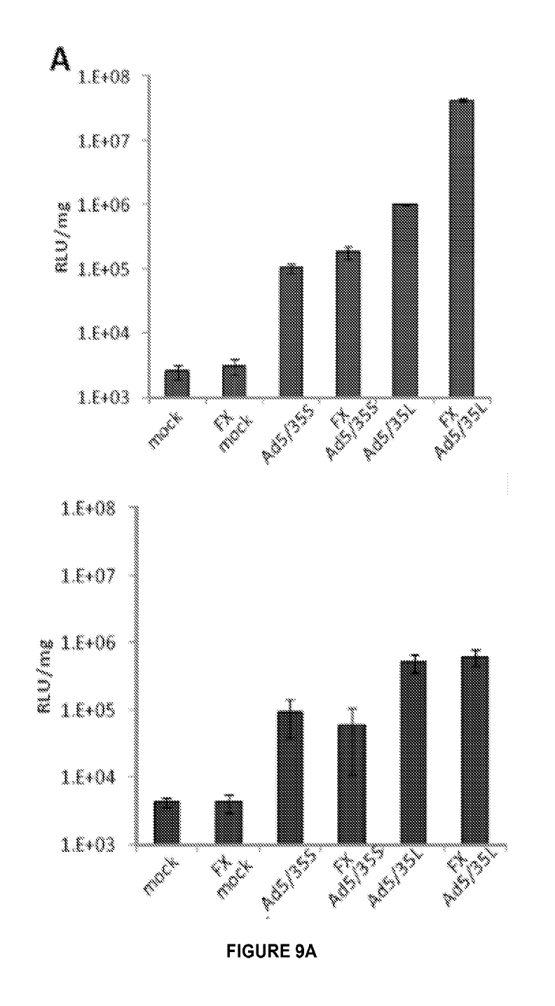

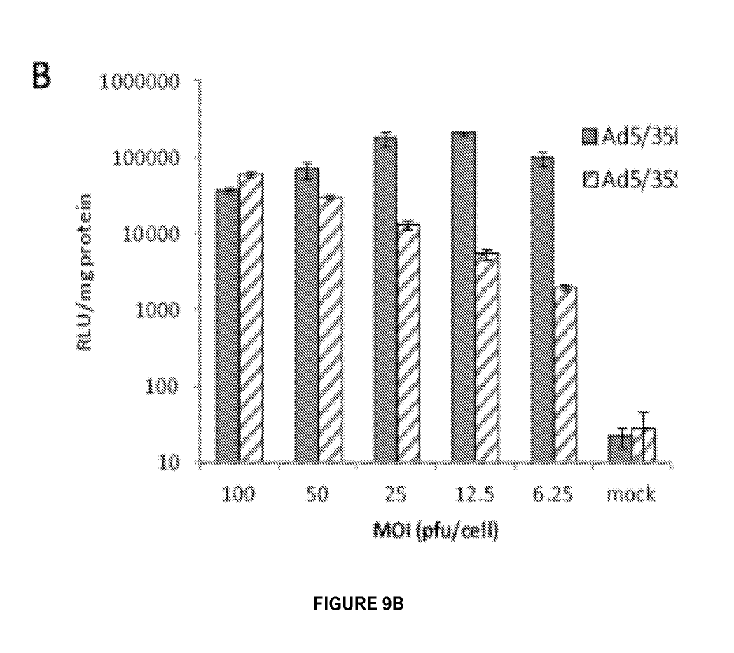

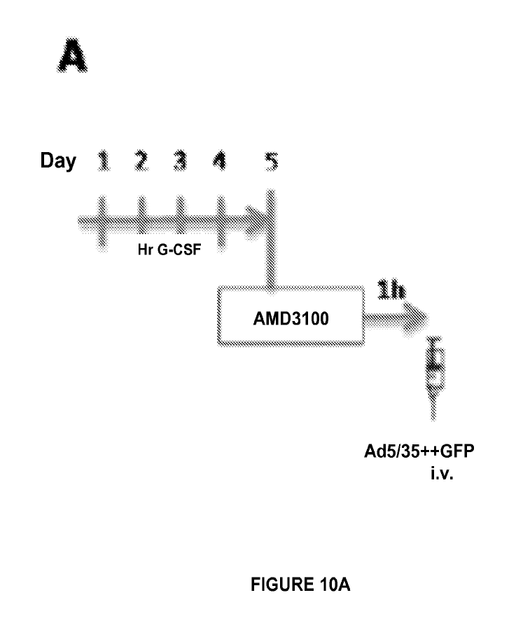

1. Ad5/35 vectors: Ad5/35 vectors contain fibers derived from human serotype Ad35. Ad5/35 and Ad35 infect cells through CD46, a receptor that is highly expressed on 100% of CD34+ cells. Absence of liver transduction by Ad5/35 vectors is important. Intravenous injection of Ad5 vectors results in hepatocyte transduction and subsequent hepatotoxicity. Ad5 entry into hepatocytes is mediated by Ad5 hexon interaction with vitamin K-dependent blood coagulation factors, specifically factor X (FX). We have shown that FX does not increase Ad5/35 transduction of CD46-negative cells (FIG. 9A.). Ad5/35 used in this study contains the Ad35 fiber shaft (w/six shaft motifs) and the Ad35 fiber knob (Ad5/35S). When the Ad35 shaft is replaced by the longer Ad5 shaft (22 shaft motifs) (Ad5/35L), FX increases the transduction of CD46-negative cells in vitro in an HSPG-dependent manner (FIG. 9A). This indicates that the shorter and less flexible Ad35 shaft interferes with FX—hexon interaction and subsequently with hepatocyte transduction. However, in vitro studies suggest that CD46-dependent transduction at low MOIs is less efficient with Ad5/35S vectors than with corresponding long-shafted Ad5/35L vectors (FIG. 9B). This is most likely due to the fact that intracellular trafficking to the cell nucleus is relatively inefficient for Ad vectors containing short fibers.

2. Affinity-enhanced Ad5/35 vectors. We constructed an Ad containing an affinity-enhanced Ad35 fiber (Ad5/35++), based on use of recombinant Ad35 fiber knobs (SEQ ID NO:100) with much improved affinity to CD46. While in humans CD46 is expressed on all nucleated cells, the corresponding orthologue in mice is expressed only in the testes. As a model for our in vivo transduction studies with intravenously injected Ad5/35 vectors, we therefore used transgenic mice that contained the complete human CD46 locus and therefore expressed huCD46 in a pattern and at a level similar to humans (huCD46tg mice). In vivo, in huCD46tg mice with pre-established CD46^{high} liver metastases, intravenous injection of Ad5/35++ resulted in >5-fold more efficient tumor cell transduction compared to the parental Ad5/35 vector.

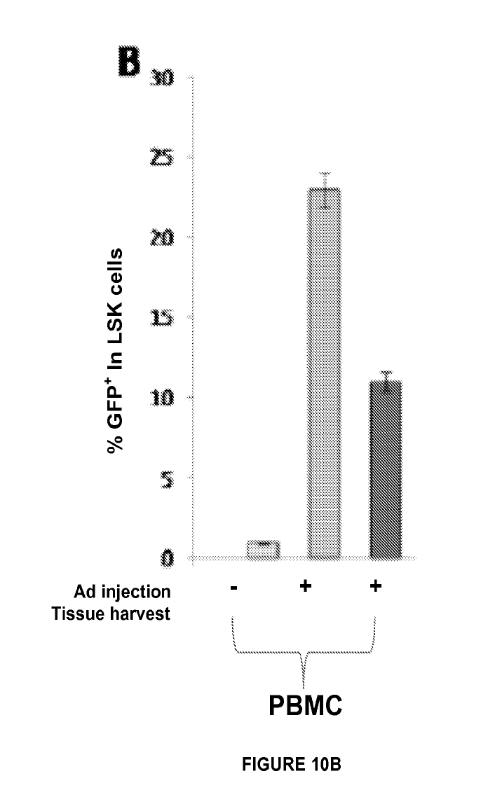

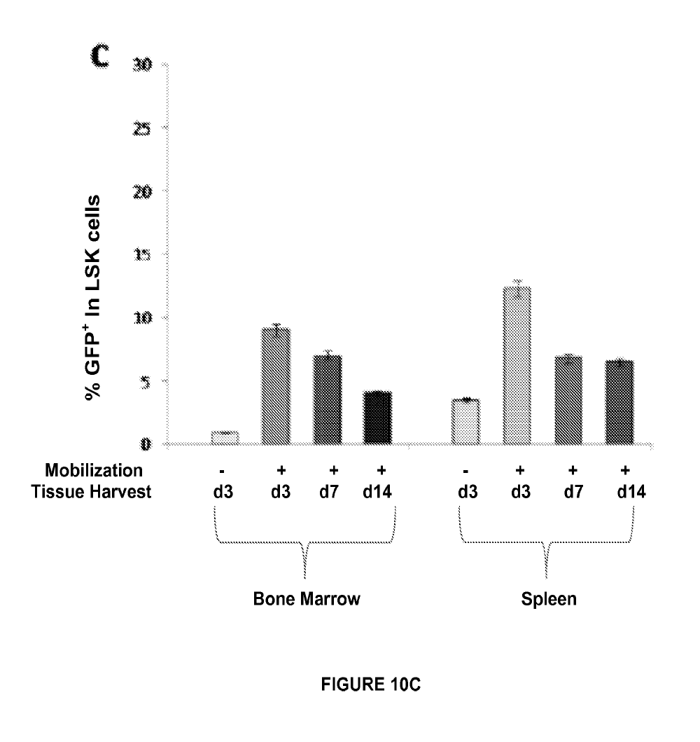

3. In vivo Ad5/35K++ transduction of mobilized HSCs. Cells localized in the bone marrow cannot be transduced by intravenously injected Ad vectors, even when the vector targets receptors that are present on bone marrow cells. This is most likely due to limited accessibility of HSCs in the bone marrow. We tested mobilization using granulocyte-colony-stimulating factor (G-CSF) and the CXCR4 antagonists AMD3100 (Mozobil™, Plerixa™) in huCD46tg mice. HSCs in mice reside within a subset of lineage-negative (Lin⁻), cKit⁺ and Seal⁺ (LSK) cells. To mobilize HSCs in huCD46tg mice, we used a combination of G-CSF and AMD3100 (FIG. 10A). G-CSF/AMD3100 mobilization resulted in a ˜100-fold increase in LSK cells in the peripheral blood at one hour after AMD3100 injection. At this time, we injected an affinity-enhanced GFP-expressing Ad5/35++ vector (122) and analyzed GFP expression in PBMCs 6 and 72 hours later (FIG. 10A and B). The study shows that more than 20% of mobilized LSK cells can be transduced in peripheral blood and that the percentage of GFP-positive LSK cells declines over time. The latter is in part due to relocalization to the bone marrow and spleen (i.e. the secondary hematopoietic organ). At day 3 post-Ad injection, about 9% and 13% of LSK cells in the bone marrow and spleen, respectively, expressed GFP. This means that 0.01% of bone marrow cells were transduced LSK cells. These numbers are therapeutically relevant if one considers that one HSC is sufficient to repopulate the complete blood system. Furthermore, we demonstrated by colony forming units (CFU) assay that transduced HSCs were pluripotent and retained the ability to form colonies. Because the Ad5/35++ vector used in this study does not integrate into the HSC genome, the number of GFP-expressing LSK cells decreased by day 14 (FIG. 10C), most likely because of cell division and cytotoxicity associated with the first-generation Ad5/35 vectors.

Next we studied Ad5/35++ in vivo transduction of human HSCs. Sublethally irradiated NOG (NOD/Shi-scid/IL-2Rγ^{null}) mice were transplanted with human CD34+ cells (NOG/CD34+ mice). Engraftment was analyzed 6 weeks later based on human CD45+ cells within PBMCs. CD45+ percentages were between 21 and 35%. NOG/CD34+ mice were than mobilized and injected with Ad5/35++-GFP as described in FIG. 2A. Forty-eight hours after Ad injection, 12 and 39% of human CD34+ cells were GFP-positive in the bone marrow cells and PBMC, respectively. Notably, the only huCD46-positive cells in this model are the transplanted human cells which might explain the higher transduction rate compared to the huCD46tg mouse model.

1. A recombinant nucleic acid expression cassette, comprising at least one first nucleic acid module comprising
(i) a first coding region encoding a nuclease capable of generating a DNA break in a CD34+ cell genomic target of interest; and
(ii) a second coding region encoding one or more miRNA target sites located in a 3′ untranslated region of the first coding region and at least 60 nucleotides downstream of a translation al stop codon of the first coding region, wherein miRNAs that bind to the one or more encoded miRNA target sites are highly expressed in virus producer cells but not expressed, or expressed at low levels, in CD34+ cells,
wherein the first nucleic acid module is operatively linked to a promoter that is active in CD34+ cells.

2. The recombinant nucleic acid expression cassette of claim 1, further comprising a second nucleic acid module encoding a CD46 binding adenoviral fiber polypeptide.

3. The recombinant nucleic acid expression cassette of claim 1, further comprising an inverted terminal repeat (ITR) at each terminus of the recombinant nucleic acid vector, wherein the ITR derived from a CD46-binding adenovirus serotype.

4. The recombinant nucleic acid expression cassette of claim 1, further comprising a packaging signal from a CD46-binding adenovirus serotype.

5. The recombinant nucleic acid expression cassette of claim 1, wherein the one or more the miRNA target site comprise a reverse complement of one, two, or all three miRNA selected from the group consisting of (a) CACUGGUAGA (SEQ ID NO: 1) (has-miR183-5p core), (b) UGUGCUUGAUCUAA (SEQ ID NO: 2) (has-miR218-5p core); and (c) CACUAGCACA (SEQ ID NO: 3) (miR96-5p core).

6. The recombinant nucleic acid expression cassette of claim 1, wherein the one or miRNA target sites comprise a reverse complement of an miRNA selected from the group consisting of SEQ ID NOS: 1-90.

7.-12. (canceled)

13. The recombinant nucleic acid expression cassette of claim 2, wherein the second nucleic acid module encodes an adenoviral fiber polypeptide comprising one or more human adenoviral knob domain, or equivalents thereof, that bind to CD46.

14. (canceled)

15. The recombinant nucleic acid expression cassette of claim 13, wherein the knob domain is selected from the group consisting of SEQ ID NOS: 94-101.

16. The recombinant nucleic acid expression cassette of claim 2, wherein the second nucleic acid module encodes an adenoviral fiber polypeptide comprising one or more human adenoviral shaft domain or functional equivalents thereof.

17. (canceled)

18. The recombinant nucleic acid expression cassette of claim 16, wherein the one or more human adenoviral shaft domains are selected from the group consisting of SEQ ID NOS: 105, 118-130, and 152-156.

19. The recombinant nucleic acid expression cassette of claim 2, wherein the second nucleic acid module encodes an adenoviral fiber polypeptide comprising a human adenoviral tail domain, or equivalent thereof.

20.-24. (canceled)

25. The recombinant nucleic acid expression cassette of claim 4, wherein the packaging signal comprises a polynucleotide selected from the group consisting of SEQ ID NO: 138-141.

26.-27. (canceled)

28. The recombinant nucleic acid expression cassette of claim 1, further comprising a transgene operatively linked to a second promoter that is active in CD34+ cells.

29. The recombinant nucleic acid expression cassette of claim 28, further comprising at least a first recombination site and a second recombination site flanking the transgene, wherein the first recombination site and a second recombination site target a site in CD34+ cell genomic DNA flanking a desired insertion site for the transgene.

30. (canceled)

31. A recombinant nucleic acid vector comprising the recombinant nucleic acid expression cassette of claim 1.

32. (canceled)

33. A recombinant host cell, comprising the expression cassette or recombinant nucleic acid vector of claim 1.

34.-36. (canceled)

37. A recombinant helper dependent adenovirus comprising the expression cassette or recombinant nucleic acid vector of claim 1.

38. A method for making a recombinant helper dependent adenovirus, comprising culturing the recombinant host cell of claim 33 under conditions suitable to promote expression of genes on the expression cassette and the helper adenovirus sufficient to assemble the helper dependent adenovirus.

39. A method for hematopoietic cell gene therapy, comprising in vivo transduction of hematopoietic cells mobilized into peripheral blood of a subject in need of hematopoietic cell gene therapy with the recombinant helper dependent Ad virus of claim 37, wherein (a) the nuclease targets a hematopoietic cell genomic gene to be disrupted, wherein disruption of the hematopoietic cell genomic gene provides a therapeutic benefit to the subject, or (b) the recombinant nucleic acid expression cassette comprises a transgene operatively linked to a promoter that is active in CD34+ cells, wherein the transgene is flanked by at least a first recombination site and a second recombination site, wherein the first recombination site and a second recombination site target a site in the hematopoietic cell genomic DNA flanking a desired insertion site for the transgene, and wherein insertion of the transgene into the desired insertion site provides a therapeutic benefit to the subject.

40.-44. (canceled)

45. A recombinant nucleic acid comprising two or more copies of a miRNA target site that comprises of the reverse complement of a nucleic acid sequence selected from the group consisting of SEQ ID NOS: 1-90.

46.-50. (canceled)


SEQUENCE LISTING (165 sequences)

SEQ ID NO: 1 - RNA, 10 nt, Artificial Sequence
  Synthetic
cacugguaga

SEQ ID NO: 2 - RNA, 14 nt, Artificial Sequence
  Synthetic
ugugcuugaucuaa

SEQ ID NO: 3 - RNA, 10 nt, Artificial Sequence
  Synthetic
cacuagcaca

SEQ ID NO: 4 - RNA, 10 nt, Artificial Sequence
  Synthetic
ucuaccagug

SEQ ID NO: 5 - RNA, 10 nt, Artificial Sequence
  Synthetic
ugugcuagug

SEQ ID NO: 6 - RNA, 14 nt, Artificial Sequence
  Synthetic
uuagaucaagcaca

SEQ ID NO: 7 - RNA, 22 nt, Artificial Sequence
  Synthetic
agugaauucuaccagugccaua

SEQ ID NO: 8 - RNA, 23 nt, Artificial Sequence
  Synthetic
agcaaaaaugugcuagugccaaa

SEQ ID NO: 9 - RNA, 21 nt, Artificial Sequence
  Synthetic
agaugguuagaucaagcacaa

SEQ ID NO: 10 - RNA, 20 nt, Artificial Sequence
  Synthetic
uguauggcacugguagaauu

SEQ ID NO: 11 - RNA, 22 nt, Artificial Sequence
  Synthetic
uguauggcacugguagaauuca

SEQ ID NO: 12 - RNA, 24 nt, Artificial Sequence
  Synthetic
uguauggcacugguagaauucacu

SEQ ID NO: 13 - RNA, 23 nt, Artificial Sequence
  Synthetic
guauggcacugguagaauucacu

SEQ ID NO: 14 - RNA, 22 nt, Artificial Sequence
  Synthetic
uauggcacugguagaauucacu

SEQ ID NO: 15 - RNA, 23 nt, Artificial Sequence
  Synthetic
uauggcacugguagaauucacug

SEQ ID NO: 16 - RNA, 20 nt, Artificial Sequence
  Synthetic
uauggcacugguagaauuca

SEQ ID NO: 17 - RNA, 21 nt, Artificial Sequence
  Synthetic
uauggcacugguagaauucac

SEQ ID NO: 18 - RNA, 19 nt, Artificial Sequence
  Synthetic
uauggcacugguagaauuc

SEQ ID NO: 19 - RNA, 24 nt, Artificial Sequence
  Synthetic
uauggcacugguagaauucacugu

SEQ ID NO: 20 - RNA, 18 nt, Artificial Sequence
  Synthetic
uauggcacugguagaauu

SEQ ID NO: 21 - RNA, 17 nt, Artificial Sequence
  Synthetic
uauggcacugguagaau

SEQ ID NO: 22 - RNA, 16 nt, Artificial Sequence
  Synthetic
uauggcacugguagaa

SEQ ID NO: 23 - RNA, 15 nt, Artificial Sequence
  Synthetic
uauggcacugguaga

SEQ ID NO: 24 - RNA, 21 nt, Artificial Sequence
  Synthetic
auggcacugguagaauucacu

SEQ ID NO: 25 - RNA, 22 nt, Artificial Sequence
  Synthetic
auggcacugguagaauucacug

SEQ ID NO: 26 - RNA, 19 nt, Artificial Sequence
  Synthetic
auggcacugguagaauuca

SEQ ID NO: 27 - RNA, 23 nt, Artificial Sequence
  Synthetic
auggcacugguagaauucacugu

SEQ ID NO: 28 - RNA, 20 nt, Artificial Sequence
  Synthetic
auggcacugguagaauucac

SEQ ID NO: 29 - RNA, 15 nt, Artificial Sequence
  Synthetic
auggcacugguagaa

SEQ ID NO: 30 - RNA, 17 nt, Artificial Sequence
  Synthetic
auggcacugguagaauu

SEQ ID NO: 31 - RNA, 18 nt, Artificial Sequence
  Synthetic
auggcacugguagaauuc

SEQ ID NO: 32 - RNA, 16 nt, Artificial Sequence
  Synthetic
auggcacugguagaau

SEQ ID NO: 33 - RNA, 21 nt, Artificial Sequence
  Synthetic
uggcacugguagaauucacug

SEQ ID NO: 34 - RNA, 19 nt, Artificial Sequence
  Synthetic
uggcacugguagaauucac

SEQ ID NO: 35 - RNA, 18 nt, Artificial Sequence
  Synthetic
cacugguagaauucacug

SEQ ID NO: 36 - RNA, 15 nt, Artificial Sequence
  Synthetic
cacugguagaauuca

SEQ ID NO: 37 - RNA, 16 nt, Artificial Sequence
  Synthetic
cacugguagaauucac

SEQ ID NO: 38 - RNA, 17 nt, Artificial Sequence
  Synthetic
cacugguagaauucacu

SEQ ID NO: 39 - RNA, 16 nt, Artificial Sequence
  Synthetic
acugguagaauucacu

SEQ ID NO: 40 - RNA, 22 nt, Artificial Sequence
  Synthetic
guugugcuugaucuaaccaugu

SEQ ID NO: 41 - RNA, 20 nt, Artificial Sequence
  Synthetic
guugugcuugaucuaaccau

SEQ ID NO: 42 - RNA, 20 nt, Artificial Sequence
  Synthetic
uugugcuugaucuaaccaug

SEQ ID NO: 43 - RNA, 19 nt, Artificial Sequence
  Synthetic
uugugcuugaucuaaccau

SEQ ID NO: 44 - RNA, 24 nt, Artificial Sequence
  Synthetic
uugugcuugaucuaaccauguggu

SEQ ID NO: 45 - RNA, 18 nt, Artificial Sequence
  Synthetic
uugugcuugaucuaacca

SEQ ID NO: 46 - RNA, 23 nt, Artificial Sequence
  Synthetic
uugugcuugaucuaaccaugugg

SEQ ID NO: 47 - RNA, 16 nt, Artificial Sequence
  Synthetic
uugugcuugaucuaac

SEQ ID NO: 48 - RNA, 21 nt, Artificial Sequence
  Synthetic
uugugcuugaucuaaccaugu

SEQ ID NO: 49 - RNA, 17 nt, Artificial Sequence
  Synthetic
uugugcuugaucuaacc

SEQ ID NO: 50 - RNA, 22 nt, Artificial Sequence
  Synthetic
uugugcuugaucuaaccaugug

SEQ ID NO: 51 - RNA, 15 nt, Artificial Sequence
  Synthetic
uugugcuugaucuaa

SEQ ID NO: 52 - RNA, 20 nt, Artificial Sequence
  Synthetic
ugugcuugaucuaaccaugu

SEQ ID NO: 53 - RNA, 21 nt, Artificial Sequence
  Synthetic
ugugcuugaucuaaccaugug

SEQ ID NO: 54 - RNA, 23 nt, Artificial Sequence
  Synthetic
ugugcuugaucuaaccauguggu

SEQ ID NO: 55 - RNA, 19 nt, Artificial Sequence
  Synthetic
gugcuugaucuaaccaugu

SEQ ID NO: 56 - RNA, 20 nt, Artificial Sequence
  Synthetic
gugcuugaucuaaccaugug

SEQ ID NO: 57 - RNA, 19 nt, Artificial Sequence
  Synthetic
ugcuugaucuaaccaugug

SEQ ID NO: 58 - RNA, 18 nt, Artificial Sequence
  Synthetic
ugcuugaucuaaccaugu

SEQ ID NO: 59 - RNA, 17 nt, Artificial Sequence
  Synthetic
gcuugaucuaaccaugu

SEQ ID NO: 60 - RNA, 16 nt, Artificial Sequence
  Synthetic
gcuugaucuaaccaug

SEQ ID NO: 61 - RNA, 15 nt, Artificial Sequence
  Synthetic
gcuugaucuaaccau

SEQ ID NO: 62 - RNA, 20 nt, Artificial Sequence
  Synthetic
gcuugaucuaaccauguggu

SEQ ID NO: 63 - RNA, 18 nt, Artificial Sequence
  Synthetic
gcuugaucuaaccaugug

SEQ ID NO: 64 - RNA, 16 nt, Artificial Sequence
  Synthetic
cuugaucuaaccaugu

SEQ ID NO: 65 - RNA, 17 nt, Artificial Sequence
  Synthetic
cuugaucuaaccaugug

SEQ ID NO: 66 - RNA, 15 nt, Artificial Sequence
  Synthetic
cuugaucuaaccaug

SEQ ID NO: 67 - RNA, 15 nt, Artificial Sequence
  Synthetic
uugaucuaaccaugu

SEQ ID NO: 68 - RNA, 16 nt, Artificial Sequence
  Synthetic
uugaucuaaccaugug

SEQ ID NO: 69 - RNA, 18 nt, Artificial Sequence
  Synthetic
uugaucuaaccauguggu

SEQ ID NO: 70 - RNA, 19 nt, Artificial Sequence
  Synthetic
uugaucuaaccaugugguu

SEQ ID NO: 71 - RNA, 17 nt, Artificial Sequence
  Synthetic
uugaucuaaccaugugg

SEQ ID NO: 72 - RNA, 24 nt, Artificial Sequence
  Synthetic
uuuuggcacuagcacauuuuugcu

SEQ ID NO: 73 - RNA, 23 nt, Artificial Sequence
  Synthetic
uuuggcacuagcacauuuuugcu

SEQ ID NO: 74 - RNA, 21 nt, Artificial Sequence
  Synthetic
uuuggcacuagcacauuuuug

SEQ ID NO: 75 - RNA, 20 nt, Artificial Sequence
  Synthetic
uuuggcacuagcacauuuuu

SEQ ID NO: 76 - RNA, 22 nt, Artificial Sequence
  Synthetic
uuuggcacuagcacauuuuugc

SEQ ID NO: 77 - RNA, 19 nt, Artificial Sequence
  Synthetic
uuuggcacuagcacauuuu

SEQ ID NO: 78 - RNA, 18 nt, Artificial Sequence
  Synthetic
uuuggcacuagcacauuu

SEQ ID NO: 79 - RNA, 15 nt, Artificial Sequence
  Synthetic
uuuggcacuagcaca

SEQ ID NO: 80 - RNA, 17 nt, Artificial Sequence
  Synthetic
uuuggcacuagcacauu

SEQ ID NO: 81 - RNA, 24 nt, Artificial Sequence
  Synthetic
uuuggcacuagcacauuuuugcuu

SEQ ID NO: 82 - RNA, 16 nt, Artificial Sequence
  Synthetic
uuuggcacuagcacau

SEQ ID NO: 83 - RNA, 21 nt, Artificial Sequence
  Synthetic
uuggcacuagcacauuuuugc

SEQ ID NO: 84 - RNA, 22 nt, Artificial Sequence
  Synthetic
uuggcacuagcacauuuuugcu

SEQ ID NO: 85 - RNA, 20 nt, Artificial Sequence
  Synthetic
ggcacuagcacauuuuugcu

SEQ ID NO: 86 - RNA, 18 nt, Artificial Sequence
  Synthetic
cacuagcacauuuuugcu

SEQ ID NO: 87 - RNA, 17 nt, Artificial Sequence
  Synthetic
cacuagcacauuuuugc

SEQ ID NO: 88 - RNA, 15 nt, Artificial Sequence
  Synthetic
acuagcacauuuuug

SEQ ID NO: 89 - RNA, 16 nt, Artificial Sequence
  Synthetic
cuagcacauuuuugcu

SEQ ID NO: 90 - RNA, 15 nt, Artificial Sequence
  Synthetic
cuagcacauuuuugc

SEQ ID NO: 91 - protein, 729 aa, Homo sapiens
MDYKDHDGDYKDHDIDYKDDDDKMAPKKKRKVGIHGVPAAMAERPFQCRICMRNFSDRSNLSRHIRTHTGEKPFACDICGRKFAISSNLNSHTKIHTGSQKPFQCRICMRNFSRSDNLARHIRTHTGEKPFACDICGRKFATSGNLTRHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINFRSGSGEGRGSLLTCGDVEENPGPRMDYKDHDGDYKDHDIDYKDDDDKMAPKKKRKVGIHGVPAAMAERPFQCRICMRNFSRSDNLSVHIRTHTGEKPFACDICGRKFAQKINLQVHTKIHTGEKPFQCRICMRNFSRSDVLSEHIRTHTGEKPFACDICGRKFAQRNHRTTHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINF

SEQ ID NO: 92 - protein, 2028 aa, Homo sapiens
MVYPYDVPDYAELPPKKKRKVGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLTPAQVVAIASNIGGKQALETVQRLLPVLCQDHGLTPDQVVAIASNIGGKQALETVQRLLPVLCQDHGLTPDQVVAIASNNGGKQALETVQRLLPVLCQAHGLTPAQVVAIASNIGGKQALETVQRLLPVLCQDHGLTPAQVVAIASNGGGKQALETVQRLLPVLCQDHGLTPDQVVAIASNIGGKQALETVQRLLPVLCQDHGLTPAQVVAIASHDGGKQALETVQRLLPVLCQDHGLTPDQVVAIASNIGGKQALETVQRLLPVLCQDHGLTPAQVVAIASNGGGKQALETVQRLLPVLCQAHGLTPAQVVAIASNNGGKQALETVQRLLPVLCQDHGLTPEQVVAIASNGGGKQALETVQRLLPVLCQAHGLTPAQVVAIASNGGGKQALETVQRLLPVLCQDHGLTPEQVVAIASNGGGKQALETVQRLLPVLCQAHGLTPDQVVAIASNGGGKQALETVQRLLPVLCQDHGLTPDQVVAIASNIGGKQALETVQRLLPVLCQDHGLTPDQVVAIASNGGGKQALETVQRLLPVLCQDHGLTPEQVVAIASNGGGKQALETVQRLLPVLCQAHGLTPDQVVAIASHDGGKQALETVQRLLPVLCQAHGLTPEQVVAIASNGGGKQALESIVAQLSRPDPALAALLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADAMQSYVEENQTRNKHINPNEWWKVYPSSVTEFKFLFVSGHFKGNYKAQLTRLNHITNCNGAVLSVEELLIGGEMIKAGTLTLEEVRRKFNNGEINFLDGSGEGRGSLLTCGDVEENPGPVYPYDVPDYAELPPKKKRKVGIRIQDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLTPDQVVAIASNIGGKQALETVQRLLPVLCQAHGLTPAQVVAIASNIGGKQALETVQRLLPVLCQDHGLTPAQVVAIASNIGGKQALETVQRLLPVLCQAHGLTPDQVVAIASHDGGKQALETVQRLLPVLCQAHGLTPDQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTPDQVVAIASNIGGKQALETVQRLLPVLCQDHGLTPDQVVAIASNGGGKQALETVQRLLPVLCQAHGLTPDQVVAIASNGGGKQALETVQRLLPVLCQAHGLTPAQVVAIASNGGGKQALETVQRLLPVLCQAHGLTPEQVVAIASNGGGKQALETVQRLLPVLCQAHGLTPEQVVAIASHDGGKQALETVQRLLPVLCQAHGLTPAQVVAIASNIGGKQALETVQRLLPVLCQAHGLTPAQVVAIASNNGGKQALETVQRLLPVLCQDHGLTPAQVVAIASNGGGKQALETVQRLLPVLCQAHGLTPDQVVAIASNNGGKQALETVQRLLPVLCQAHGLTPAQVVAIASHDGGKQALETVQRLLPVLCQAHGLTPEQVVAIASNGGGKQALESIVAQLSRPDPALAALLVKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQAREMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFLD

SEQ ID NO: 93 - protein, 844 aa, Macaca fuscata
MDYKDHDGDYKDHDIDYKDDDDKMAPKKKRKVGIHGVPAAMAERPFQCRICMRNFSRSDNLSVHIRTHTGEKPFACDICGRKFAANHHRINHTKIHTGSQKPFQCRICMRNFSDRSDLSRHIRTHTGEKPFACDICGRKFARSDHLSRHTKIHTGSQKPFQCRICMRNFSQSGNLARHIRTHTGEKPFACDICGRKFAQRNDRKSHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINFRSGSGEGRGSLLTCGDVEENPGPRMDYKDHDGDYKDHDIDYKDDDDKMAPKKKRKVGIHGVPAAMAERPFQCRICMRNFSRSDHLSQHIRTHTGEKPFACDICGRKFATSANRTTHTKIHTGSQKPFQCRICMRNFSERGTLARHIRTHTGEKPFACDICGRKFAQSSDLRRHTKIHTGSQKPFQCRICMRNFSQSSDLSRHIRTHTGEKPFACDICGRKFACRSNLKKHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINF

SEQ ID NO: 94 - protein, 191 aa, Artificial Sequence
  Synthetic
WTGVNPTEANCQIMNSSESNDCKLILTLVKTGALVTAFVYVIGVSNNFNMLTTHRNINFTAELFFDSTGNLLTRLSSLKTPLNHKSGQNMATGAITNAKGFMPSTTAYPFNDNSREKENYIYGTCYYTASDRTAFPIDISVMLNRRAINDETSYCIRITWSWNTGDAPEVQTSATTLVTSPFTFYYIREDD

SEQ ID NO: 95 - protein, 190 aa, Artificial Sequence
  Synthetic
WTGAKPSANCVIKEGEDSPDCKLTLVLVKNGGLINGYITLMGASEYTNTLFKNNQVTIDVNLAFDNTGQIITYLSSLKSNLNFKDNQNMATGTITSAKGFMPSTTAYPFITYATETLNEDYIYGECYYKSTNGTLFPLKVTVTLNRRMLASGMAYAMNFSWSLNAEEAPETTEVTLITSPFFFSYIREDD

SEQ ID NO: 96 - protein, 189 aa, Artificial Sequence
  Synthetic
WTGIKPPPNCQIVENTDTNDGKLTLVLVKNGGLVNGYVSLVGVSDTVNQMFTQKSATIQLRLYFDSSGNLLTDESNLKIPLKNKSSTATSEAATSSKAFMPSTTAYPFNTTTRDSENYIHGICYYMTSYDRSLVPLNISIMLNSRTISSNVAYAIQFEWNLNAKESPESNIATLTTSPFFFSYIREDDN

SEQ ID NO: 97 - protein, 189 aa, Artificial Sequence
  Synthetic
WTGINPPPNCQIVENTNTNDGKLTLVLVKNGGLVNGYVSLVGVSDTVNQMFTQKTANIQLRLYFDSSGNLLTDESDLKIPLKNKSSTATSETVASSKAFMPSTTAYPFNTTTRDSENYIHGICYYMTSYDRSLFPLNISIMLNSRMISSNVAYAIQFEWNLNASESPESNIATLTTSPFFFSYITEDDN

SEQ ID NO: 98 - protein, 189 aa, Artificial Sequence; identical to SEQ ID NO: 96
  Synthetic

SEQ ID NO: 99 - protein, 189 aa, Artificial Sequence
  Synthetic
  MISC_FEATURE (73)..(73): X is D or G
  MISC_FEATURE (111)..(111): X is T or A
  MISC_FEATURE (122)..(122): X is I or L
WTGINPPPNCQIVENTNTNDGKLTLVLVKNGGLVNGYVSLVGVSDTVNQMFTQKTANIQLRLYFDSSGNLLTXESDLKIPLKNKSSTATSETVASSKAFMPSTTAYPFNTXTRDSENYIHGXCYYMTSYDRSLFPLNISIMLNSRMISSNVAYAIQFEWNLNASESPESNIATLTTSPFFFSYITEDDN

SEQ ID NO: 100 - protein, 189 aa, Artificial Sequence; identical to SEQ ID NO: 97
  Synthetic

SEQ ID NO: 101 - protein, 189 aa, Artificial Sequence
  Synthetic
WTGINPPPNCQIVENTNTNDGKLTLVLVKNGGLVNGYVSLVGVSDTVNQMFTQKTANIQLRLYFDSSGNLLTGESDLKIPLKNKSSTATSETVASSKAFMPSTTAYPFNTATRDSENYIHGLCYYMTSYDRSLFPLNISIMLNSRMISSNVAYAIQFEWNLNASESPESNIATLTTSPFFFSYITEDDN

SEQ ID NO: 102 - protein, 24 aa, Artificial Sequence
  Synthetic
FPPEVEEQDDGTLPMSCAQEGMDR

SEQ ID NO: 103 - protein, 751 aa, Artificial Sequence
  Synthetic
MGHPHPDKLQKGGGSGGGSGGGSDYKDHDGDYKDHDIDYKDDDDKMAPKKKRKVGIHGVPAAMAERPFQCRICMRNFSDRSNLSRHIRTHTGEKPFACDICGRKFAISSNLNSHTKIHTGSQKPFQCRICMRNFSRSDNLARHIRTHTGEKPFACDICGRKFATSGNLTRHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRNKHLNPNEWWKVYPSSVTEFKFLFVSGHFKGNYKAQLTRLNHITNCNGAVLSVEELLIGGEMIKAGTLTLEEVRRKFNNGEINFRSGSGEGRGSLLTCGDVEENPGPRMDYKDHDGDYKDHDIDYKDDDDKMAPKKKRKVGIHGVPAAMAERPFQCRICMRNFSRSDNLSVHIRTHTGEKPFACDICGRKFAQKINLQVHTKIHTGEKPFQCRICMRNFSRSDVLSEHIRTHTGEKPFACDICGRKFAQRNHRTTHTKIHLRGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVKENQTRNKHINPNEWWKVYPSSVTEFKFLFVSGHFKGNYKAQLTRLNHKTNCNGAVLSVEELLIGGEMIKAGTLTLEEVRRKFNNGEINF

SEQ ID NO: 104 - DNA, 387 bp, Artificial Sequence
  Synthetic
aagtttccagagctttcgaggaaggtttcttcaactcaaattcatccgcctgataattttcttatattttcctaaagaaggaagagaagcgcatagaggagaagggaaataattttttaggagcctttcttacggctatgaggaatttggggctcagttgaaaagcctaaactgcctctcgggaggttgggcgcggcgaactactttcagcggcgcacggagacggcgtctacgtgaggggtgataagtgacgcaacactcgttgcataaatttgcgctccgccagcccggagcatttaggggcggttggctttgttgggtgagcttgtttgtgtccctgtgggtggacgtggttggtgattggcaggatcctggtatccgctacag

SEQ ID NO: 105 - protein, 358 aa, Artificial Sequence
  Synthetic
PGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTL

SEQ ID NO: 106 - protein, 100 aa, Artificial Sequence
  Synthetic
ELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQ

SEQ ID NO: 107-117 - skipped (11 entries)

SEQ ID NO: 118 - protein, 92 aa, Artificial Sequence
  Synthetic
NGVLTLKCLTPLTTTGGSLQLKVGGGLTVDDTNGFLKENISATTPLVKTGHSIGLPLGAGLGTNENKLCIKLGQGLTFNSNNICIDDNINTL

SEQ ID NO: 119 - protein, 121 aa, Artificial Sequence
  Synthetic
DGVLTLKCVNPLTTASGPLQLKVGSSLTVDTIDGSLEENITAAAPLTKTNHSIGLLIGSGLQTKDDKLCLSLGDGLVTKDDKLCLSLGDGLITKNDVLCAKLGHGLVFDSSNAITIENNTL

SEQ ID NO: 120 - protein, 92 aa, Artificial Sequence
  Synthetic
DGVLTLNCLTPLTTTGGPLQLKVGGGLIVDDTDGTLQENIRATAPITKNNHSVELSIGNGLETQNNKLCAKLGNGLKFNNGDICIKDSINTL

SEQ ID NO: 121 - protein, 92 aa, Artificial Sequence
  Synthetic
DGVLTLKCLTPLTTTGGSLQLKVGGGLTVDDTDGTLQENIRATAPITKNNHSVELSIGNGLETQNNKLCAKLGNGLKFNNGDICIKDSINTL

SEQ ID NO: 122 - protein, 92 aa, Artificial Sequence
  Synthetic
DGVLTLNCLTPLTTTGGPLQLKVGGGLIVDDTDGTLQENIRVTAPITKNNHSVELSIGNGLETQNNKLCAKLGNGLKFNNGDICIKDSINTL

SEQ ID NO: 123 - protein, 91 aa, Artificial Sequence
  Synthetic
  MISC_FEATURE (4)..(4): X is T or S
  MISC_FEATURE (8)..(8): X is L or V
  MISC_FEATURE (9)..(9): X is T or N
  MISC_FEATURE (14)..(14): X is T or A
  MISC_FEATURE (15)..(15): X is G or S
  MISC_FEATURE (24)..(24): X is G or S
  MISC_FEATURE (30)..(30): X is D or T
  MISC_FEATURE (32)..(32): X is D or N
  MISC_FEATURE (34)..(34): X is T, F or S
  MISC_FEATURE (36)..(36): X is Q, K or E
  MISC_FEATURE (40)..(40): X is G, S or K
  MISC_FEATURE (41)..(41): X is A or V
  MISC_FEATURE (42)..(42): X is T or N
  MISC_FEATURE (46)..(46): X is T or V
  MISC_FEATURE (48)..(48): X is T or S
  MISC_FEATURE (49)..(49): X is G or N
  MISC_FEATURE (53)..(53): X is G or N
  MISC_FEATURE (55)..(55): X is S or P
  MISC_FEATURE (56)..(56): X is L or I
  MISC_FEATURE (58)..(58): X is A, P, or N
  MISC_FEATURE (61)..(61): X is G or Q
  MISC_FEATURE (62)..(62): X is T or I
  MISC_FEATURE (63)..(63): X is D or E
  MISC_FEATURE (64)..(64): X is Q or E
  MISC_FEATURE (69)..(69): X is T, S, or A
  MISC_FEATURE (73)..(73): X is E, Q, or N
  MISC_FEATURE (78)..(78): X is D or N
  MISC_FEATURE (80)..(80): X is S or N
  MISC_FEATURE (82)..(82): X is S or I
  MISC_FEATURE (83)..(83): X is C or I
  MISC_FEATURE (84)..(84): X is A or I
  MISC_FEATURE (85)..(85): X is D, N, or L
  MISC_FEATURE (86)..(86): X is D or K
  MISC_FEATURE (88)..(88): X is I or absent
GVLXLKCXXPLTTXXGSLQLKVGXGLTVDXTXGXLXENIXXXTPLXKXXHSIXLXXGXGLXXXXNKLCXKLGXGLTFXSXNXXXXXNXNTL

SEQ ID NO: 124 - protein, 10 aa, Artificial Sequence
  Synthetic
NSIALKNNTL

SEQ ID NO: 125 - protein, 14 aa, Artificial Sequence
  Synthetic
NSNNICINDNINTL

SEQ ID NO: 126 - protein, 15 aa, Artificial Sequence
  Synthetic
GAITVGNKNNDKLTL

SEQ ID NO: 127 - protein, 14 aa, Artificial Sequence
  Synthetic
NSNNICIDDNINTL

SEQ ID NO: 128 - protein, 14 aa, Artificial Sequence
  Synthetic
NSNNICIDDNINTL

SEQ ID NO: 129 - protein, 12 aa, Artificial Sequence
  Synthetic
GDICIKDSINTL

SEQ ID NO: 130 - protein, 91 aa, Artificial Sequence
  Synthetic
  MISC_FEATURE (28)..(28): X is V or I
  MISC_FEATURE (34)..(34): X is T or F
  MISC_FEATURE (36)..(36): X is Q or K
  MISC_FEATURE (40)..(40): X is G or S
  MISC_FEATURE (55)..(55): X is P or S
  MISC_FEATURE (58)..(58): X is P or A
  MISC_FEATURE (63)..(63): X is D or N
  MISC_FEATURE (69)..(69): X is T or A
  MISC_FEATURE (73)..(73): X is E or Q
  MISC_FEATURE (85)..(85): X is D or N
GVLTLKCLTPLTTTGGSLQLKVGGGLTXDDTDGXLXENIXATTPLVKTGHSIGLXLGXGLGTXENKLCXKLGXGLTFNSNNICIXDNINTL

SEQ ID NO: 131 - protein, 42 aa, Artificial Sequence
  Synthetic
MTKRVRLSDSFNPVYPYEDESTSQHPFINPGFISPNGFTQSP

SEQ ID NO: 132 - protein, 42 aa, Artificial Sequence
  Synthetic
MAKRARLSSSFNPVYPYEDESSSQHPFINPGFISSNGFAQSP

SEQ ID NO: 133 - DNA, 137 bp, Artificial Sequence
  Synthetic
catcatcaataatataccttatagatggaatggtgccaatatgtaaatgaggtgattttaaaaagtgtggatcgtgtggtgattggctgtggggttaacggctaaaaggggcggtgcgaccgtgggaaaatgacgtt

SEQ ID NO: 134 - DNA, 114 bp, Artificial Sequence
  Synthetic
cattatctataatataccttatagatggaatggtgccaacatgtaaatgaggtaatttaaaaaagtgcgcgctgtgtggtgattggctgcggggtgaacggctaaaaggggcgg

SEQ ID NO: 135 - DNA, 112 bp, Artificial Sequence
  Synthetic
tattatataatataccttatagatggaatggtgccaatatgcaaatgaggtaatttaaaaaagtgcgcgctgtgtggtgattggctgcggggtgaacggctaaaaggggcgg

SEQ ID NO: 136 - DNA, 137 bp, Artificial Sequence
  Synthetic
catcatcaataatataccttatagatggaatggtgccaatatgtaaatgaggtgattttaaaaagtgtgggccgtgtggtgattggctgtggggttaacggttaaaaggggcggcgcggccgtgggaaaatgacgtt

SEQ ID NO: 137 - DNA, 114 bp, Artificial Sequence
  Synthetic
caatcaatataatataccttatagatggaatggtgccaatatgtaaatgaggtaatttaaaaaagtgcgcgctgtgtggtgattggctgcggggtgaacggctaaaaggggcgg

SEQ ID NO: 138 - DNA, 541 bp, Artificial Sequence
  Synthetic
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggatccaataacttcgtatagcatacattatacgaagttataagtactgaattcggatccggtgtacacaggaagtgacttttaacgcgcggtttgttacggatgttgtagtaaatttgtctagggccgagtaagatttgaccgtttacgcggggactttgaataagagcgagtgaaatctgaataattttgttgtactcatagcgcgtaatctctagacgttatataattcgaatttgtacccggggatccaataacttcgtatagcatacattatacgaagttataagtactgaattcggatcctctagctagagtcgacctgcaggcatgctctagactcgagggatct

SEQ ID NO: 139 - DNA, 180 bp, Artificial Sequence
  Synthetic
gtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgacc

SEQ ID NO: 140 - DNA, 210 bp, Artificial Sequence
  Synthetic
acgcataaaaaggcttcttttctcacggaactacttagttttcccacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggagg

SEQ ID NO: 141 - DNA, 178 bp, Artificial Sequence
  Synthetic
ctgtacacacgaaacgacaattttcccacggtattaaacagaaaatgaagtaaatttgaccggaaccaagtaaaaattgcccattttcgcgcgaaaactgaatgaggaagtgaaatctgaataatgtggtattaatcacagcgcggaatatttgtccagggccacggagactttgacc

SEQ ID NO: 142 - DNA, 114 bp, Artificial Sequence
  Synthetic
  misc_feature (1)..(6): optionally absent
  misc_feature (105)..(114): optionally absent
ctaggattatggcactggtagaattcactacttatggcactggtagaattcactacttatggcactggtagaattcactacttatggcactggtagaattcactatcgcccggg

SEQ ID NO: 143 - DNA, 115 bp, Artificial Sequence
  Synthetic
  misc_feature (1)..(9): optionally absent
  misc_feature (106)..(115): optionally absent
cctaggaatttgtgcttgatctaaccatgtttcattgtgcttgatctaaccatgtttcattgtgcttgatctaaccatgtttcattgtgcttgatctaaccatgtatcgcccggg

SEQ ID NO: 144 - DNA, 215 bp, Artificial Sequence
  Synthetic
  misc_feature (1)..(8): optionally absent
  misc_feature (206)..(215): optionally absent
cctaggattatggcactggtagaattcactacttatggcactggtagaattcactacttatggcactggtagaattcactacttatggcactggtagaattcactatcgttgtgcttgatctaaccatgtttcattgtgcttgatctaaccatgtttcattgtgcttgatctaaccatgtttcattgtgcttgatctaaccatgtatcgcccggg

SEQ ID NO: 145 - DNA, 513 bp, Artificial Sequence
  Synthetic
cacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctcccca

SEQ ID NO: 146 - DNA, 1335 bp, Artificial Sequence
  Synthetic
gagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctcgccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 147 - DNA, 114 bp, Artificial Sequence; identical to SEQ ID NO: 142
  Synthetic

SEQ ID NO: 148 - DNA, 115 bp, Artificial Sequence; identical to SEQ ID NO: 143
  Synthetic

SEQ ID NO: 149 - DNA, 215 bp, Artificial Sequence; identical to SEQ ID NO: 144
  Synthetic

SEQ ID NO: 150 - DNA, 24 bp, Artificial Sequence
  Synthetic
aaatcttgaccattctccactctc

SEQ ID NO: 151 - DNA, 24 bp, Artificial Sequence
  Synthetic
ggagacacacagaaatgtaacagg

SEQ ID NO: 152 - protein, 91 aa, Artificial Sequence
  Synthetic
NGVLSLKCVNPLTTASGSLQLKVGSGLTVDTTDGSLEENIKVNTPLTKSNHSINLPIGNGLQIEQNKLCSKLGNGLTFDSSNSIALKNNTL

SEQ ID NO: 153 - protein, 92 aa, Artificial Sequence
  Synthetic
DGVLTLKCLTPLTTTGGSLQLKVGGGLTIDDTDGFLKENISATTPLVKTGHSIGLSLGPGLGTNENKLCAKLGEGLTFNSNNICINDNINTL

SEQ ID NO: 154 - protein, 92 aa, Artificial Sequence; identical to SEQ ID NO: 118
  Synthetic

SEQ ID NO: 155 - protein, 91 aa, Artificial Sequence
  Synthetic
GVLTLKCLTPLTTTGGSLQLKVGGGLTVDDTDGTLQENIGATTPLVKTGHSIGLSLGAGLGTDENKLCTKLGEGLTFNSNNICIDDNINTL

SEQ ID NO: 156 - protein, 92 aa, Artificial Sequence
  Synthetic
DGVLTLKCLTPLTTTGGSLQLKVGGGLTVDDTDGTLQENIGATTPLVKTGHSIGLSLGAGLGTDENKLCTKLGEGLTFNSNNICIDDNINTL

SEQ ID NO: 157 - RNA, 23 nt, Artificial Sequence
  Synthetic
uggaagacuagugauuuuguugu

SEQ ID NO: 158 - RNA, 23 nt, Artificial Sequence
  Synthetic
uaaggugcaucuagugcagauag

SEQ ID NO: 159 - RNA, 44 nt, Artificial Sequence
  Synthetic
cagugcaauguuaaaagggcaucagugcaauguuaaaagggcau

SEQ ID NO: 160 - DNA, 104 bp, Artificial Sequence
  Synthetic
ctgttattttttacttggactcttgtggggaataagatacatgttttattcttatttatgattcaagcactgaaaatagtgtttagcatccagcaggtgcttca

SEQ ID NO: 161 - DNA, 80 bp, Artificial Sequence
  Synthetic
ctgttattttttacttggactcttgtggggaataagatacatgcactgaaaatagtgtttagcatccagcaggtgcttca

SEQ ID NO: 162 - protein, 590 aa, Artificial Sequence
  Synthetic
QKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTGINPPPNCQIVENTNTNDGKLTLVLVKNGGLVNGYVSLVGVSDTVNQMFTQKTANIQLRLYFDSSGNLLTEESDLKIPLKNKSSTATSETVASSKAFMPSTTAYPFNTTTRDSENYIHGICYYMTSYDRSLFPLNISIMLNSRMISSNVAYAIQFEWNLNASESPESNIATLTTSPFFFSYITEDDN

SEQ ID NO: 163 - protein, 729 aa, Artificial Sequence; identical to SEQ ID NO: 91
  Synthetic

SEQ ID NO: 164 - protein, 2028 aa, Artificial Sequence; identical to SEQ ID NO: 92
  Synthetic

SEQ ID NO: 165 - protein, 844 aa, Artificial Sequence; identical to SEQ ID NO: 93
  Synthetic